SEQUENCE LISTING (109 sequences)

SEQ ID NO: 1 - protein, 829 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 2 - protein, 839 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 849 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 4 - protein, 772 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 782 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 6 - protein, 792 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 7 - protein, 754 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 8 - protein, 764 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - protein, 774 aa, Artificial Sequence
  source: /note=“Description of Artificial Sequence       Synthetic polypeptide”
MGTPAQLLFLLLLWLPDTTGQVQLQQSGPELKKPGETVKISCKASGYTFTNYGMNWVKQAPGRGLKWMGWINTYTGESTYADDFKGRFAFSLETSASAAYLQINNLKNEDTATYFCARFAIKGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAGAGGGGSGGGGSGGGGSSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 10 - protein, 239 aa, Artificial Sequence
  source: /note=“Description of Artificial Sequence       Synthetic polypeptide”
MGTPAQLLFLLLLWLPDTTGDIVMTQSAFSNPVTLGTSASISCRSTKSLLHSNGITYLYWYLQKPGQSPQLLIYQMSNLASGVPDRFSSSGSGTDFTLRISRVEAEDVGVYYCAQNLEIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 801 aa, Artificial Sequence
  source: /note=“Description of Artificial Sequence       Synthetic polypeptide”
MGTPAQLLFLLLLWLPDTTGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 12 - DNA, 664 bp, Artificial Sequence
  source: /note=“Description of Artificial Sequence       Synthetic polynucleotide”
gaacgcgtggagggggtaagcctatacctaacccgctgttggggttagacagcacgggtggatccgtcagagaaagaggtccacaaagagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtatcttcgaattgaaggaaaacgacagaatcttcgtttcagtcaccaacgaacatttgattgatatggaccacgaagcatcctttttcggtgcctttttagtaggtggaacacaatagcaattacagggcgcctcaggatctggtgactacaaggacgacgatgacaagggtaccggcgggtccggagctagtgccaaaag

SEQ ID NO: 13 - DNA, 718 bp, Artificial Sequence
  source: /note=“Description of Artificial Sequence       Synthetic polynucleotide”
gttctaggtctcatgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaagagaccattgtg

SEQ ID NO: 14 - DNA, 30 bp, Artificial Sequence
  source: /note=“Description of Artificial Sequence       Synthetic primer”
gaacgcgtggagggggtaagcctataccta

SEQ ID NO: 15 - DNA, 25 bp, Artificial Sequence
  source: /note=“Description of Artificial Sequence       Synthetic primer”
cttttggcactagctccggacccgc

SEQ ID NO: 16 - DNA, 69 bp, Artificial Sequence
  source: /note=“Description of Artificial Sequence       Synthetic primer”
tacctaacccgctgttggggttagacagcacgggtggatccgtcagagaaagaggtccacaaagagtcg

SEQ ID NO: 17 - DNA, 91 bp, Artificial Sequence
  source: /note=“Description of Artificial Sequence       Synthetic primer”
ttgtcatcgtcgtccttgtagtcaccagatcctgaggcgccctgtaattgctattgtgttccacctactaaaaaggcaccgaaaaaggatg

SEQ ID NO: 18 - DNA, 20 bp, Artificial Sequence
  source: /note=“Description of Artificial Sequence       Synthetic primer”
tacctaacccgctgttgggg

SEQ ID NO: 19 - DNA, 23 bp, Artificial Sequence
  source: /note=“Description of Artificial Sequence       Synthetic primer”
ttgtcatcgtcgtccttgtagtc

SEQ ID NO: 20 - DNA, 42 bp, Artificial Sequence
  source: /note=“Description of Artificial Sequence       Synthetic primer”
gttctaggtctcatgtgggctgataagacacatacatgccct

SEQ ID NO: 21 - DNA, 40 bp, Artificial Sequence
  source: /note=“Description of Artificial Sequence       Synthetic primer”
cacaatggtctcttcctccacccggcgacaagcttagcga

SEQ ID NO: 22 - DNA, 36 bp, Artificial Sequence
  source: /note=“Description of Artificial Sequence       Synthetic primer”
gttctaggtctcaaggaggcggcagtggtggaggtg

SEQ ID NO: 23 - DNA, 39 bp, Artificial Sequence
  source: /note=“Description of Artificial Sequence       Synthetic primer”
cacaatggtctctaccaccgcccaccagaaaggcaccga

SEQ ID NO: 24 - DNA, 36 bp, Artificial Sequence
  source: /note=“Description of Artificial Sequence       Synthetic primer”
gttctaggtctcatggtggcggcagtggtggaggtg

SEQ ID NO: 25 - DNA, 39 bp, Artificial Sequence
  source: /note=“Description of Artificial Sequence       Synthetic primer”
cacaatggtctctcccgccgcccaccagaaaggcaccga

SEQ ID NO: 26 - DNA, 36 bp, Artificial Sequence
  source: /note=“Description of Artificial Sequence       Synthetic primer”
gttctaggtctcacgggggcggcagtggtggaggtg

SEQ ID NO: 27 - DNA, 37 bp, Artificial Sequence
  source: /note=“Description of Artificial Sequence       Synthetic primer”
cacaatggtctctattagcccaccagaaaggcaccga

SEQ ID NO: 28 - protein, 281 aa, Homo sapiens [sequence omitted]

SEQ ID NO: 29 - DNA, 85 bp, Artificial Sequence
  source: /note=“Description of Artificial Sequence       Synthetic primer”
ggagagggtctcgaggaggcggcagtggtggaggtggatctggcggaggaggctctgtcagagaaagaggtccacaaagagtcgc

SEQ ID NO: 30 - DNA, 74 bp, Artificial Sequence
  source: /note=“Description of Artificial Sequence       Synthetic primer”
tctctcggtctccactaccgccacctcctgatcctccaccgccacctactaaaaaggcaccgaaaaaggatgct

SEQ ID NO: 31 - DNA, 61 bp, Artificial Sequence
  source: /note=“Description of Artificial Sequence       Synthetic primer”
gagagaggtctcgtagtggtggcggaggttcagtcagagaaagaggtccacaaagagtcgc

SEQ ID NO: 32 - DNA, 74 bp, Artificial Sequence
  source: /note=“Description of Artificial Sequence       Synthetic primer”
tctctcggtctcctgagcctcctccgccactgccaccgcctccacctactaaaaaggcaccgaaaaaggatgct

SEQ ID NO: 33 - DNA, 61 bp, Artificial Sequence
  source: /note=“Description of Artificial Sequence       Synthetic primer”
gagagaggtctcgctcaggcggaggtggcagtgtcagagaaagaggtccacaaagagtcgc

SEQ ID NO: 34 - DNA, 48 bp, Artificial Sequence
  source: /note=“Description of Artificial Sequence       Synthetic primer”
tctctcggtctccattaacctactaaaaaggcaccgaaaaaggatgct

SEQ ID NO: 35 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 36 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 37 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 38 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 39 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 40 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVG

SEQ ID NO: 41 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 42 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 43 - protein, 775 aa, Artificial Sequence; identical to SEQ ID NO: 35
  source: /note=“Description of Artificial Sequence       Synthetic polypeptide”

SEQ ID NO: 44 - protein, 775 aa, Artificial Sequence; identical to SEQ ID NO: 37
  source: /note=“Description of Artificial Sequence       Synthetic polypeptide”

SEQ ID NO: 45 - protein, 775 aa, Artificial Sequence; identical to SEQ ID NO: 36
  source: /note=“Description of Artificial Sequence       Synthetic polypeptide”

SEQ ID NO: 46 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 47 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 48 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 49 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 50 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 51 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtcagagaaagaggtccacaaatagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggcggtggaggatcaggaggtggcggtagtggtggcggaggttcagtcagagaaagaggtccacaaatagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggaggcggtggcagtggcggaggaggctcaggcggaggtggcagtgtcagagaaagaggtccacaaatagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggt

SEQ ID NO: 52 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtcagagaaagaggtccacaaagagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaagctcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggcggtggaggatcaggaggtggcggtagtggtggcggaggttcagtcagagaaagaggtccacaaagagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaagctcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggaggcggtggcagtggcggaggaggctcaggcggaggtggcagtgtcagagaaagaggtccacaaagagtcgccgcccacataacaggtacaagaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaagctcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggt

SEQ ID NO: 53 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtcagagaaagaggtccacaaagagtcgccgcccacataacaggtacaggaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggcggtggaggatcaggaggtggcggtagtggtggcggaggttcagtcagagaaagaggtccacaaagagtcgccgcccacataacaggtacaggaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggtggaggcggtggcagtggcggaggaggctcaggcggaggtggcagtgtcagagaaagaggtccacaaagagtcgccgcccacataacaggtacaggaggtagaagtaacacattaagttccccaaatagtaagaatgaaaaagctttgggtagaaagattaactcttgggaatcttcaagatccggtcattcatttttgtctaatttgcacttaagaaacggtgaattagtcattcatgaaaagggtttctactacatctattctcaaacatacttcagattccaagaagaaattaaagaaaacaccaaaaacgataagcaaatggtacaatacatctataagtacacaagttatccagaccctatcttgttgatgaagtctgcaagaaactcatgttggtccaaggatgccgaatacggtttgtactctatctatcaaggtggtgtcttcgaattgaaggaaaacgacagaatcttcgtttcagtcaccaacgaacatttgattgatatggaccacgaagcatcctttttcggtgcctttttagtaggt

SEQ ID NO: 54 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 55 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 56 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagcgtgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatggaccgactacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 57 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 58 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 59 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 60 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagcgggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 61 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgaggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgaggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgaggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtgtgtttgaactgaaagaaaacgatcgcattttcgtgtctgttaccaatgaacatctgatcgatatggatcacgaagcgagctttttcggtgcctttctggtgggc

SEQ ID NO: 62 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtaacagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggc

SEQ ID NO: 63 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggc

SEQ ID NO: 64 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagatcgttgcagcccatattaccggcacgcggggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggc

SEQ ID NO: 65 - DNA, 2388 bp, Artificial Sequence
  source: /note=“Description of Artificial Sequence       Synthetic polynucleotide”
atggcctggcggctgtggtggctgctgctcctgctcctgttgctttggcctatggtgtgggctgataagacacatacatgccctccatgtcccgcacccgagttgcttggaggaccttcggtgtttctttttcccccgaagccaaaagatacactgatgatttcacggacgcccgaggtgacttgtgtcgtcgtggacgtcagccacgaggacccagaagtcaagtttaactggtatgtagatggggtggaggtacacaatgcgaaaacgaaaccgagagaggagcagtacaattcgacgtatagggtggtcagcgtgctgacggtgttgcaccaggactggctgaacgggaaagagtataagtgcaaagtgtcgaacaaggccctccccgcacccatcgaaaagacgatatccaaagccaagggccaaccgcgcgagccgcaagtgtacacgctgcctccctcgcgagaagagatgaccaagaaccaggtgtcccttacgtgcttggtgaaaggattctacccttcggacatcgccgtagaatgggaaagcaatgggcagccagagaacaattacaaaaccacaccgcctgtgctcgactcggacggttcctttttcttgtattccaagttgacagtggacaagtcacggtggcaacaggggaacgtattctcgtgttccgtcatgcacgaagcgctgcataaccactacactcagaagtcgctaagcttgtcgccgggtggaggaggcggcagtggtggaggtggatctggcggaggaggctctgtgcgtgaacgtggtccgcagagggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggtggtggcggcagtggtggaggtggatctggcggaggaggctctgtgcgtgaacgtggtccgcagagggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggcggcgggggcggcagtggtggaggtggatctggcggaggaggctctgtgcgtgaacgtggtccgcagagggttgcagcccatattaccggcacgggcggtcgctctaacacgctgagctctccgaacagtaaaaatgaaaaagcgctgggccgtaaaatcaattcttgggaaagtagccgcagcggtcattcttttctgagtaacctgcacctgcgtaatggcgaactggtgatccacgaaaaaggtttctactacatctacagccagacctactttcgcttccaggaagaaatcaaagaaaacacgaaaaacgataaacagatggttcagtacatctacaaatacaccagctacccggacccgatcctgctgatgaaatctgcgcgtagcagctgctggtctaaagatgccgaatacggcctgtatagcatttaccagggcggtatctttgaactgaaagaaaacgatcgcattttcgtgtctgttaccaatgaacatctgatcgatatggatcacgaagcgagctttttcggtgcctttctggtgggc

SEQ ID NO: 66 - protein, 534 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 67 - protein, 534 aa, Artificial Sequence
  source: /note=“Description of Artificial Sequence       Synthetic polypeptide”
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 68 - protein, 534 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 69 - protein, 534 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 70 - protein, 534 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 71 - protein, 534 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVG

SEQ ID NO: 72 - protein, 534 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 73 - protein, 534 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 74 - protein, 534 aa, Artificial Sequence; identical to SEQ ID NO: 66
  source: /note=“Description of Artificial Sequence       Synthetic polypeptide”

SEQ ID NO: 75 - protein, 534 aa, Artificial Sequence; identical to SEQ ID NO: 68
  source: /note=“Description of Artificial Sequence       Synthetic polypeptide”

SEQ ID NO: 76 - protein, 534 aa, Artificial Sequence; identical to SEQ ID NO: 67
  source: /note=“Description of Artificial Sequence       Synthetic polypeptide”

SEQ ID NO: 77 - protein, 534 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 78 - protein, 534 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 79 - protein, 534 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 80 - protein, 534 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 81 - protein, 534 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVGGGGGSGGGGSGGGGSVRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 82 - protein, 168 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 83 - protein, 168 aa, Artificial Sequence
  source: /note=“Description of Artificial Sequence       Synthetic polypeptide”
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 84 - protein, 168 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 85 - protein, 168 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 86 - protein, 168 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGVFELKENDRIFVSVTNEHLIDMDHEASFFGAFLVG

SEQ ID NO: 87 - protein, 168 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARSSCWSKDAEYGLYSIYQGGVFELKENDRIFVSVTNEHLIDMDHEASFFGAFLVG

SEQ ID NO: 88 - protein, 168 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 89 - protein, 168 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 90 - protein, 168 aa, Artificial Sequence; identical to SEQ ID NO: 82
  source: /note=“Description of Artificial Sequence       Synthetic polypeptide”

SEQ ID NO: 91 - protein, 168 aa, Artificial Sequence; identical to SEQ ID NO: 84
  source: /note=“Description of Artificial Sequence       Synthetic polypeptide”

SEQ ID NO: 92 - protein, 168 aa, Artificial Sequence; identical to SEQ ID NO: 83
  source: /note=“Description of Artificial Sequence       Synthetic polypeptide”

SEQ ID NO: 93 - protein, 168 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGVFELKENDRIFVSVTNEHLIDMDHEASFFGAFLVG

SEQ ID NO: 94 - protein, 168 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 95 - protein, 168 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 96 - protein, 168 aa, Artificial Sequence
  source: /note=“Description of Artificial Sequence       Synthetic polypeptide”
VRERGPQIVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 97 - protein, 168 aa, Artificial Sequence
  source: /note=“Description of Artificial Sequence       Synthetic polypeptide”
VRERGPQRVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARSSCWSKDAEYGLYSIYQGGIFELKENDRIFVSVTNEHLIDMDHEASFFGAFLVG

SEQ ID NO: 98 - protein, 1015 aa, Artificial Sequence
  source: /note=“Description of Artificial Sequence       Synthetic polypeptide”
MGTPAQLLFLLLLWLPDTTGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 99 - protein, 995 aa, Artificial Sequence
  source: /note=“Description of Artificial Sequence       Synthetic polypeptide”
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 100 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 101 - protein, 775 aa, Artificial Sequence
  source: /note=“Description of Artificial Sequence       Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 102 - protein, 534 aa, Artificial Sequence
  source: /note=“Description of Artificial Sequence       Synthetic polypeptide”
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVGGGGGSGGGGSGGGGSVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 103 - protein, 534 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVGGGGGSGGGGSGGGGSVRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 104 - protein, 168 aa, Artificial Sequence
  source: /note=“Description of Artificial Sequence       Synthetic polypeptide”
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 105 - protein, 168 aa, Artificial Sequence
  source: /note=“Description of Artificial Sequence       Synthetic polypeptide”
VRERGPQIVAAHITGTGGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGVFELKENDRIFVSVTNEHLIDMDHEASFFGAFLVG

SEQ ID NO: 106 - protein, 15 aa, Artificial Sequence
  source: /note=“Description of Artificial Sequence       Synthetic peptide”
GGGGSGGGGSGGGGS

SEQ ID NO: 107 - protein, 6 aa, Artificial Sequence
  source: /note=“Description of Artificial Sequence       Synthetic 6xHis tag”
HHHHHH

SEQ ID NO: 108 - protein, 5 aa, Artificial Sequence
  source: /note=“Description of Artificial Sequence       Synthetic peptide”
GGGGS

SEQ ID NO: 109 - protein, 10 aa, Artificial Sequence
  source: /note=“Description of Artificial Sequence       Synthetic peptide”
GGGGSGGGGS

(19) United States
(12) Patent Application Publication
(10) Pub. No.: US 2019/0077870 A1
(43) Pub. Date: Mar. 14, 2019

(54) ENGINEERED TRAIL FOR CANCER THERAPY

(71) Applicant: Merrimack Pharmaceuticals, Inc., Cambridge, MA (US)

(72) Inventors: Diana Hung-yi Chai MARCANTONIO, Concord, MA (US); Stephen L. SAZINSKY, Melrose, MA (US); Birgit M. SCHOEBERL, Cambridge, MA (US); Eric M. TAM, Cambridge, MA (US)

(21) Appl. No.: 16/084,447

(22) PCT Filed: Mar. 16, 2017
(86) PCT No.: PCT/US2017/022789

§ 371 Date: Sep. 12, 2018

Related U.S. Application Data
(60) Provisional application No. 62/445,556, filed on Jan. 12, 2017.
(60) Provisional application No. 62/309,352, filed on Mar. 16, 2016.
(60) Provisional application No. 62/323,501, filed on Apr. 15, 2016.

(51) Int. Cl. C07K 16/28; A61K 38/17; C12N 15/62; A61P 35/00; C07K 14/705

(52) U.S. Cl. CPC C07K 16/2878; A61K 38/17; C07K 14/70575; A61P 35/00; C12N 15/62

(57) ABSTRACT

Engineered single chain trail molecules are provided, as are particular mutations and combinations of mutations that improve the stability and manufacturability of such molecules. These molecules are provided for use as anti-cancer therapeutics.

### RELATED APPLICATIONS

This application claims priority to U.S. provisional Application No. 62/309,352 filed Mar. 16, 2016, 62/323,501 filed Apr. 15, 2016, and 62/445,556 filed Jan. 12, 2017. The contents of the aforementioned applications are hereby incorporated by reference.

### INTRODUCTION

Apo2L/TRAIL (TNF-related apoptosis-inducing ligand, CD253) is a member of the TNF family that binds and activates the death receptors (specifically DR4 and DR5). TRAIL also binds non-signaling decoy receptors, DcR1, DcR2, and osteoprotegrin (OPG). TRAIL naturally occurs as a type 2 transmembrane protein, with an extracellular domain that can be cleaved to release a soluble trimeric protein. Clustering of the receptor complex, e.g., as mediated by the trimeric structure of TRAIL, is necessary for efficient signaling and induction of apoptosis by the death receptors. Additionally, higher order oligomerization of receptor complexes can amplify signaling, resulting in greater induction of apoptosis.

Soluble recombinant TRAIL has been produced and tested as a cancer therapeutic. It has a short half-life in humans (approximately 0.5-1 hours) (Herbst et al., Journal of Clinical Oncology, 2010 Jun. 10; 28(17):2839-46), which has presumably limited its efficacy.

In addition, expression of TRAIL ligand is difficult. Recombinant TRAIL constructs are unstable and are characterized by low melting temperature (Tₘ) and the propensity to form misfolded aggregates, which can result in unwanted toxicities (Lawrence et al, Nature Medicine, 2001 April; 7(4):383-5). Stabilization of the trimer has been attempted mainly through N-terminal fusion to a scaffold domain (e.g., modified leucine zipper or trimerization domain of tenascin-C.) (Walczak et al., Nature Medicine, 1999 February; 5(2):157-63). These stabilization domains may result in added immunogenicity, limiting their utility in therapeutics. A single-chain fusion polypeptide of TRAIL, connected by peptide linkers, has been described as an alternative method of producing trimerized TRAIL (Schneider et al., Cell Death & Disease, 2010 Aug. 26; 1:e68). However, we have observed that this molecule is not suited for clinical development as it displays instabilities characterized by aggregation, low thermal melting temperature, and/or loss of activity when incubated in serum.

One of the major shortcomings of previous attempts to prepare a recombinant human TRAIL as a therapeutic is short serum half-life (Table 1).

TABLE 1

| | | Terminal | |
|---|---|---|---|
| | Dose | half-life | |
| Species | (mg/kg) | (min) | Reference |
| Nude mice | 10 | 3.6 | Kelley et al., Journal of |
| | | | Pharmacology and |
| | | | Experimental Therapeutics, |
| | | | 2001 Oct; 299(1): 31-8 |
| Cynomolgus | 1, 5, 50 | 23.0-31.2 | Kelley et al., Journal of |
| monkey | | | Pharmacology and |
| | | | Experimental Therapeutics, |
| | | | 2001 Oct; 299(1): 31-8 |
| Human | 0.5, 1.5, 4, 8, | 0.6-1.0 | Herbst et al., Journal of |
| | 15, 20, 30 | | Clinical Oncology, 2010 |
| | | | Jun 10; 28(17): 2839-46 |

It has been shown that a single polypeptide chain variant of TRAIL, in which peptide linkers were used to connect adjacent TRAIL monomers, had slightly improved its serum half-life (T_{1/2}˜35 minutes) and bioactivity (Schneider et al., Cell Death & Disease, 2010 Aug. 26; 1:e68). However the improved T₁₁₂ was still too short for effective clinical use.

Therefore, there remains a need for a death receptor agonist that can be expressed, purified, has sufficient stability for commercial manufacture and distribution, and retains biological activity in vivo. The present disclosure addresses this need and provides additional advantages.

### BRIEF DESCRIPTION OF THE DRAWINGS

FIG. 1A: Representation of Fab-scTRAIL. The scTRAIL (grey) is fused to the C terminus of the anti-EpCAM MOC31 heavy chain (white). The light chain of MOC31 is indicated as hatched. The single disulfide bond between the constant domains of the Fab (straight line) and the glycine-serine linkers connecting the Fab to a TRAIL monomer and connecting the TRAIL monomers to each other (curved lines) are also shown.

FIG. 1B: scTRAIL variants (T1-T9) are represented in a matrix of TRAIL sequence lengths and glycine serine linker lengths. Figure discloses SEQ ID NOS 108-109 and 106, respectively, in order of appearance.

FIG. 1C: SDS-PAGE analysis of T1-T9 variants (2 μg) under non-reducing and reducing conditions.

FIGS. 1D-1L: Size exclusion chromatography of T1-T9 variants using a TSKgel® SuperSW3000 column. The percentage of major peak is indicated.

FIGS. 2A-2C: Activity of Fab-scTRAIL variants in a cell viability assay using HeLa cells. Cells were treated for 24 hours with increasing concentrations of T1-T9. Cell viability was determined by measuring ATP levels and plotted as function of protein concentration.

FIG. 3: Cartoon representation of a homodimer of scTRAIL (grey) fused to the C terminus of human IgG1 Fc (white). The disulfide bonds of the hinge region and the GS linkers connecting TRAIL monomers are also shown.

FIG. 4: Size exclusion chromatography of Fc-scTRAIL using a TSKgel® SuperSW3000. SDS-PAGE analysis of Fc-scTRAIL (1 μg) under non-reducing and reducing conditions.

FIGS. 5A-5D: Activity of Fc-scTRAIL in cell viability assays using COLO205 (FIG. 5A), HCT116 (FIG. 5B), DU145 cells (FIG. 5C), and Jurkat cells (FIG. 5D). Cells were treated for 24 hours with increasing concentrations of Fc-scTRAIL, TRAIL, and agonistic DR4 and DR5 antibodies. Cell viability was determined by measuring ATP levels and plotted as function of protein concentration.

FIGS. 6A-B: Activity of agonistic DR4 and DR5 antibodies and Fc-scTRAIL in cell viability assays using Jurkat cells. In FIG. 6A, cells were treated for 24 hours with increasing concentrations of anti-DR4 (open square), cross-linked anti-DR4 (closed squares), anti-DR5 (open circles), and cross-linked anti-DR5 (closed circles). In FIG. 6B, cells were treated for 24 hours with increasing concentration of cross-linked anti-DR4, cross-linked anti-DR5, the combination of cross-linked anti-DR4 and 5, and Fc-scTRAIL. Cell viability was determined by measuring ATP levels and plotted as function of protein concentration.

FIGS. 7A-C: Activity of agonistic DR4 and DR5 antibodies and Fc-scTRAIL in cell viability assays using DU145 (FIG. 7A), COLO205 (FIG. 7B), and PANC1 (FIG. 7C) cells. Cells were treated for 24 hours with increasing concentrations of cross-linked anti-DR4 (closed square), cross-linked anti-DR5 (closed triangles), cross-linked anti-DR4 and 5 (closed circles), and Fc-scTRAIL (open circles). Cell viability was determined by measuring ATP levels and plotted as function of protein concentration.

FIG. 8: Activity of Fc-scTRAIL and Fc-scTRAIL Q variants in cell viability assays using H1993 cells. Cells were treated for 24 hours with increasing concentrations of Fc-scTRAIL (circle), Fc-scTRAIL Q1 (diamond), Fc-scTRAIL Q2 (squares), Fc-scTRAIL Q3 (triangles). Cell viability was determined by measuring ATP levels and plotted as function of protein concentration.

FIGS. 9A-9B: (FIG. 9A) Thermal melt curves for TRAIL and Fc-scTRAIL. (FIG. 9B) Activity of Fc-scTRAIL following 0, 3, and 7 day serum incubation. HCT116 cells were treated for 24 hours with increasing concentration of serum incubated Fc-scTRAIL and cell viability was determined by measuring ATP levels and plotted as function of protein concentration.

FIGS. 10A-10C: Flow cytometric analysis of yeast library panning. Cells were labeled with biotin-DR5-Fc (10 nM) and anti-FLAG (2 μg/ml) followed by SA/Alexa647 and anti-mouse/Alexa 488. Fluorescence was measured and represented in a bivariate plot. (FIG. 10A) Unselected library. (FIG. 10B) Enriched population after 4 rounds of panning. (FIG. 10C) Exemplary clone overlaid with wild-type TRAIL.

FIG. 11: Amino acid substitutions and thermal melt curves for Fc-scTRAIL mutants, T148, T151, and T153.

FIGS. 12A-12D: Cell viability assays of serum incubated Fc-scTRAIL (FIG. 12A) and Fc-scTRAIL mutants (FIGS. 12B-12D). HCT116 cells treated for 24 hours with serum incubated T148, T151, and T153. Cell viability curves are shown for 0, 3 and 7 day incubated samples.

FIG. 13: Amino acid substitutions and thermal melt curves for Fc-scTRAIL mutants, T183, T186, and T191.

FIGS. 14A-14D: Cell viability assays of serum incubated Fc-scTRAIL (FIG. 14A) and Fc-scTRAIL mutants (FIGS. 14B-14D). HCT116 cells treated for 24 hours with serum incubated T183 (FIG. 14C), T186 (FIG. 14B), and T191 (FIG. 14D). Cell viability curves are shown for 0, 3 and 7 day incubated samples.

FIGS. 15A-15E: Cell viability assay of PANC-1 (FIG. 15A), DU145 (FIG. 15B), A549 (FIG. 15C), SK-LU-1 (FIG. 15D) and HOP62 (FIG. 15E) cells. Cells were treated for 24 hours with increasing concentrations of T191 and TRAIL. Cell viability was determined by measuring ATP levels and plotted as function of protein concentration. Solid circles indicate TRAIL, open circles indicate T191.

FIG. 16: The effect of Fc-mediated cross-linking on the activity of T191 as measured in a cell viability assay using DU145 cells. Cells were treated for 24 hours with increasing concentrations of T191 with (solid circles) or without (open circles) an equimolar concentration of anti-Fc antibody. Cell viability was determined by measuring ATP levels and plotted as function of protein concentration.

FIG. 17: Time course of T191-induced apoptosis. DU145 cells were treated with 10 nM T191 for 2, 4, 8, or 24 hours with and without anti-Fc cross-linking. Cell lysates were probed by western blot for caspase-8 (55/53, 43/41, 18 kDa), Bid (22, 15 kDa), PARP (116, 89 KDa), and GAPDH (37 kDa). Activation (cleavage) of caspase-8, BH3 interacting-domain death agonist (BID), and PARP is observed as early as 2 hours after T191 treatment. Clearance of caspase-8 and BID is also observed at later time points.

FIG. 18: Pharmacokinetics of T191. Shown here for the 5 mg/kg dose are the functional T191 levels in serum at different time-points (n=3) measured using DR5 ELISA and plotted as function of time. Biexponential fit and 95% confidence intervals are indicated.

FIG. 19: Comparison of TRAIL and T191 efficacy in the COLO205 xenograft model. Nude mice were injected subcutaneously with COLO205 cells and dosed with PBS, TRAIL or T191. Plotted are mean tumors volumes as a function of time with the standard error represented as error bars. Statistical differences between treatment groups (p<0.005) are indicated by (*).

FIGS. 20A-20B: Efficacy of T191 in the HCC2998 (FIG. 20A) and LS411N (FIG. 20B) xenograft models. Nude mice were injected subcutaneously with HCC2998 and LS411N cells and dosed with PBS (squares) or T191 (circles) on days 5 and 12 (arrows) post inoculation. Plotted are mean tumor volumes as a function of time with the standard error represented as error bars.

FIG. 21: Schematic representation of anti-EpcAM IgG-scTRAIL (grey) is fused to the C terminus of the MOC-31 IgG heavy chain (white). The light chain of MOC31 IgG is indicated as hatched. The disulfide bonds between heavy and light chain constant regions and between the hinge region are indicated by straight line. Glycine-serine linkers between MOC-31 IgG and scTRAIL and between TRAIL monomers are shown curved lines.

FIG. 22: Kinetics of cell viability for MOC-31 IgG-scTRAIL across a panel of cancer cell lines. Cells were treated for 0.5, 1, 2, 4, 8 and 24 hours with increasing concentrations of TRAIL and MOC-31 IgG-scTRAIL. Cell viability was determined by measuring ATP levels and visualized as a heat map. Within each cell line, an individual square represents a single molecule concentration and time point, relative to control (untreated cells at time point zero). Cell viability is indicated by blue (100%) and red (0%) colors.

FIG. 23: Caspase 8 activation of MOC-31 IgG-scTRAIL in HCT116 cells. Cells were treated for 0.5, 1, 2, 4, 8 and 24 hours with 41 pM of TRAIL or MOC-31 IgG-scTRAIL. Active caspase 8 levels were measured and normalized to untreated control before being plotted as function of time.

FIG. 24: Activity of MOC-31 IgG-scTRAIL as measured in a cell viability assay using HCT116 cells. Cells were treated for 24 hours with increasing concentrations of Fc-scTRAIL and MOC-31 IgG-scTRAIL. Cell viability was determined by measuring ATP levels and plotted as function of protein concentration.

### SUMMARY

Provided herein are single mutant polypeptide chains of an Fc-TRAIL fusion polypeptide comprised of two polypeptide chains dimerized by at least one inter-Fc disulfide bond.

Also provided are other TRAIL fusion polypeptides that provide increased half-life in circulating blood in a human patient. These include TRAIL trimers, Fc-TRAIL fusions, TRAIL—antibody Fab fragment fusions and TRAIL albumin fusions.

In one embodiment, the mutant chain comprises a human IgG Fc moiety peptide-bound to a set of three human TRAIL monomer moieties to form a single unbranched polypeptide.

In another embodiment, the a single unbranched polypeptide comprises, in amino- to carboxyl-terminal order, an Fc moiety, a TRAIL-Fc linker, a first TRAIL monomer, an inter-TRAIL monomer linker, a second TRAIL monomer, a second inter-TRAIL monomer linker, and a third TRAIL monomer.

In another embodiment, each linker consists of 15-20 amino acids.

In another embodiment, each of the two inter-TRAIL monomer linkers comprises 3 G₄S domains (SEQ ID NO: 106).

In another embodiment, at least two of the three TRAIL monomers comprises at least one stabilizing mutation not found in native wild-type human TRAIL.

In another embodiment, the Fc-TRAIL fusion polypeptide formed by the dimerization of two copies of the mutant polypeptide chain exhibits a melting temperature of greater than or equal to 65° C.

In another embodiment, the at least one stabilizing mutation is at a corresponding to position 247 of wild-type TRAIL (SEQ ID NO:28) and is an amino acid other than an isoleucine that is located at this position in wild-type TRAIL. In another embodiment, the amino acid other than the isoleucine is glycine, alanine, valine or leucine.

In a particular embodiment, the single mutant polypeptide chain of an Fc-TRAIL fusion polypeptide comprises two polypeptide chains dimerized by at least one inter-Fc disulfide bond, the mutant chain comprising a human IgG Fc moiety peptide-bound to a set of three human TRAIL monomer moieties to form a single unbranched polypeptide comprising, in amino- to carboxyl-terminal order, the Fc moiety, a TRAIL-Fc linker, a first TRAIL monomer, an inter-TRAIL monomer linker, a second TRAIL monomer, a second inter-TRAIL monomer linker, and a third TRAIL monomer, wherein the each linker consists of 15-20 amino acids and each of the two inter-TRAIL monomer linkers comprises 3 G₄S domains (SEQ ID NO: 106), and wherein at least two of the three TRAIL monomers comprises at least one stabilizing mutation not found in native wild-type human TRAIL, and wherein, the Fc-TRAIL fusion polypeptide formed by the dimerization of two copies of the mutant polypeptide chain exhibits a melting temperature of greater than or equal to 65° C.

In one embodiment, a single mutant polypeptide chain of a TRAIL fusion polypeptide comprises a human serum albumin moiety peptide-bound to a set of three human TRAIL monomer moieties to form a single unbranched polypeptide comprising, in amino- to carboxyl-terminal order, the Fc moiety, a TRAIL-Fc linker, a first TRAIL monomer, an inter-TRAIL monomer linker, a second TRAIL monomer, a second inter-TRAIL monomer linker, and a third TRAIL monomer, wherein the each linker consists of 15-20 amino acids and each of the two inter-TRAIL monomer linkers comprises 3 G₄S domains (SEQ ID NO: 106), and wherein at least two of the three TRAIL monomers comprises at least one stabilizing mutation not found in native wild-type human TRAIL. In another embodiment, the fusion polypeptide is formed by the dimerization of two copies of the mutant polypeptide chain and exhibits a melting temperature of greater than or equal to 65° C.

Also provided herein are methods of treating a cancer in a human patient, the method comprising administering to the patient an effective amount of a TRAIL fusion polypeptide (e.g., Fc-TRAIL fusion polypeptide) as described herein.

In one embodiment, the treatment method comprises administering to the patient an effective amount of the Fc-TRAIL fusion polypeptide formed by the dimerization of two copies of the mutant polypeptide chain.

In another embodiment, the treatment methods described herein comprise administering a TRAIL fusion polypeptide (e.g., Fc-TRAIL fusion polypeptide) in combination with one or more other antineoplastic agents (e.g., other chemotherapeutics or other small molecule drugs). In another embodiment, no more than three other antineoplastic agents are administered within the treatment cycle. In another embodiment, no more than two other antineoplastic agents are administered within the treatment cycle. In another embodiment, no more than one other antineoplastic agent is administered within the treatment cycle. In another embodiment, no other antineoplastic agent is administered within the treatment cycle.

As used herein, adjunctive or combined administration (coadministration) includes simultaneous administration of a TRAIL fusion polypeptide (e.g., Fc-TRAIL fusion polypeptide) and one or more antineoplastic agents in the same or different dosage form, or separate administration of the TRAIL fusion polypeptide and one or more antineoplastic agents (e.g., sequential administration). Such concurrent or sequential administration preferably results in both the TRAIL fusion polypeptide and the one or more agents being simultaneously present in treated patients.

In another embodiment, the patient is selected for treatment with a TRAIL fusion polypeptide based on an FDA-approved test.

Also provided herein are polypeptides comprising an amino acid sequence that is at least 95% identical to amino acid residues 95-281, 114-281, or 120-281 of SEQ ID NO: 28. In one embodiment, the polypeptide comprises a substitution at one or more of position 121, 130, 228, and 247. In another embodiment, the polypeptide comprises at least one substitution selected from the group consisting of R121I, R130G, N228S, and I247V. In another embodiment, the polypeptide comprises at least one substitution selected from the group consisting of I247G, I247A, I247V, and I 247L. In another embodiment, the polypeptide comprises a substitution at one or both of positions 213 and 215. In another embodiment, the polypeptide comprises at least one substitution selected from the group consisting of Y213W and S215D. In another embodiment, the polypeptide comprises a set of substitutions selected from the group consisting of: (i) R121I and I247V; (ii) N228S and I247V; (iii) R130G and I247V; (iv) R121I, R130G, Y213W, S215D and I247V; (v) R130G, Y213W, S215D and I247V; and (vi) R130G, Y213W, S215D, N228S and I247V. In another embodiment, the polypeptide comprises a set of substitutions selected from the group consisting of: (i) R121I, R130G, and I247V; (ii) R130G, N228S, and I247V; (iii) R121I, R130G, N228S, and I247V; (iv) R121I, N228S, and I247V; (v) R121I and R130G; (vi) R121I, R130G, and N228S; (vii) R121I and N228S; and (viii) R130G and N228S.

In another embodiment, the polypeptide comprises a sequence selected from the group consisting of SEQ ID NO: 82, 83, 84, 85, 86, 87, 88, 89, 90, 91, 92, 93, 94, 95, 96, and 97. In another embodiment, the polypeptide comprises the sequence set forth in SEQ ID NO: 82. In another embodiment, the polypeptide comprises the sequence set forth in SEQ ID NO: 83. In another embodiment, the polypeptide comprises the sequence set forth in SEQ ID NO: 84. In another embodiment, the polypeptide comprises the sequence set forth in SEQ ID NO: 85. In another embodiment, the polypeptide comprises the sequence set forth in SEQ ID NO: 86. In another embodiment, the polypeptide comprises the sequence set forth in SEQ ID NO: 87. In another embodiment, the polypeptide comprises the sequence set forth in SEQ ID NO: 88. In another embodiment, the polypeptide comprises the sequence set forth in SEQ ID NO: 89. In another embodiment, the polypeptide comprises the sequence set forth in SEQ ID NO: 90. In another embodiment, the polypeptide comprises the sequence set forth in SEQ ID NO: 91. In another embodiment, the polypeptide comprises the sequence set forth in SEQ ID NO: 92. In another embodiment, the polypeptide comprises the sequence set forth in SEQ ID NO: 93. In another embodiment, the polypeptide comprises the sequence set forth in SEQ ID NO: 94. In another embodiment, the polypeptide comprises the sequence set forth in SEQ ID NO: 95. In another embodiment, the polypeptide comprises the sequence set forth in SEQ ID NO: 96. In another embodiment, the polypeptide comprises the sequence set forth in SEQ ID NO: 97.

Also provided herein are polypeptides comprising a set of three human TRAIL monomer moieties to form a single-chain TRAIL trimer. In one embodiment, the single-chain TRAIL trimer comprises, in amino- to carboxyl-terminal order, a first TRAIL monomer, an inter-TRAIL monomer linker, a second TRAIL monomer, a second inter-TRAIL monomer linker, and a third TRAIL monomer. In another embodiment, each linker consists of 15-20 amino acids. In another embodiment, each of the two inter-TRAIL monomer linkers comprises 3 G₄S domains (SEQ ID NO: 106). In another embodiment, at least two of the three TRAIL monomers comprises at least one stabilizing mutation not found in native wild-type human TRAIL. In another embodiment, the at least one stabilizing mutation is at a corresponding to position 247 of wild-type TRAIL (SEQ ID NO:28) and is an amino acid other than an isoleucine that is located at this position in wild-type TRAIL. In another embodiment, the amino acid other than the isoleucine is glycine, alanine, valine or leucine.

In another embodiment, the polypeptide comprises an amino acid sequence that is at least 95% identical to amino acid residues 95-281, 114-281, or 120-281 of SEQ ID NO: 28. In one embodiment, the polypeptide comprises a substitution at one or more of position 121, 130, 228, and 247 of SEQ ID: 28. In another embodiment, the polypeptide comprises at least one substitution selected from the group consisting of R121I, R130G, N228S, and I247V of SEQ ID NO: 28. In another embodiment, the polypeptide comprises at least one substitution selected from the group consisting of I247G, I247A, I247V, and I 247L. In another embodiment, the polypeptide comprises a substitution at one or both of positions 213 and 215 of SEQ ID: 28. In another embodiment, the polypeptide comprises at least one substitution selected from the group consisting of Y213W and S215D. In another embodiment, the polypeptide comprises a set of substitutions selected from the group consisting of: (i) R121I and I247V; (ii) N228S and I247V; (iii) R130G and I247V; (iv) R121I, R130G, Y213W, S215D and I247V; (v) R130G, Y213W, S215D and I247V; and (vi) R130G, Y213W, S215D, N228S and I247V. In another embodiment, the polypeptide comprises a set of substitutions selected from the group consisting of: (i) R121I, R130G, and I247V; (ii) R130G, N228S, and I247V; (iii) R121I, R130G, N228S, and I247V; (iv) R121I, N228S, and I247V; (v) R121I and R130G; (vi) R121I, R130G, and N228S; (vii) R121I and N228S; and (viii) R130G and N228S.

In another embodiment, the polypeptide comprises a sequence selected from the group consisting of SEQ ID NO: 66, 67, 68, 69, 70, 71, 72, 73, 74, 75, 76, 77, 78, 79, 80, and 81. In another embodiment, the polypeptide comprises the sequence set forth in SEQ ID NO: 66. In another embodiment, the polypeptide comprises the sequence set forth in SEQ ID NO: 67. In another embodiment, the polypeptide comprises the sequence set forth in SEQ ID NO: 68. In another embodiment, the polypeptide comprises the sequence set forth in SEQ ID NO: 69. In another embodiment, the polypeptide comprises the sequence set forth in SEQ ID NO: 70. In another embodiment, the polypeptide comprises the sequence set forth in SEQ ID NO: 71. In another embodiment, the polypeptide comprises the sequence set forth in SEQ ID NO: 72. In another embodiment, the polypeptide comprises the sequence set forth in SEQ ID NO: 73. In another embodiment, the polypeptide comprises the sequence set forth in SEQ ID NO: 74. In another embodiment, the polypeptide comprises the sequence set forth in SEQ ID NO: 75. In another embodiment, the polypeptide comprises the sequence set forth in SEQ ID NO: 76. In another embodiment, the polypeptide comprises the sequence set forth in SEQ ID NO: 77. In another embodiment, the polypeptide comprises the sequence set forth in SEQ ID NO: 78. In another embodiment, the polypeptide comprises the sequence set forth in SEQ ID NO: 79. In another embodiment, the polypeptide comprises the sequence set forth in SEQ ID NO: 80. In another embodiment, the polypeptide comprises the sequence set forth in SEQ ID NO: 81.

Also provided herein are proteins comprising two polypeptide chains, each polypeptide chain comprises a portion of an antibody constant region and a single-chain TRAIL trimer, wherein the protein has a melting temperature greater than about 60° C. (e.g., each of 61-77° C.). In one embodiment the protein has a melting temperature of 60, 61, 62, 63, 64, 65, 66, 67, 68, 69, 70, or 71° C. In another embodiment, the melting temperature is measured by differential scanning fluorometry.

In one embodiment, the TRAIL trimer comprises a set of three human TRAIL monomer moieties. In another embodiment, the polypeptide chain comprises an amino acid sequence at least 95% identical to amino acid residues 95-281, 114-281, or 120-281 of SEQ ID NO:28, and comprises a substitution at one or more of positions 121, 130, 228, and 247. In another embodiment, the polypeptide chain comprises an amino acid sequence at least 96%, 97%, 98%, or 99% identical to amino acid residues 95-281, 114-281, or 120-281 of SEQ ID NO:28.

In another embodiment, the polypeptide chain comprises a sequence selected from the group consisting of T148 (SEQ ID NO: 35), T151 (SEQ ID NO: 36), T153 (SEQ ID NO: 37), T183 (SEQ ID NO: 38), T186 (SEQ ID NO: 39), T191 (SEQ ID NO: 40), T202 (SEQ ID NO: 41), T203 (SEQ ID NO: 42), T204 (SEQ ID NO: 43), T205 (SEQ ID NO: 44), T206 (SEQ ID NO: 45), T207 (SEQ ID NO: 46), T208 (SEQ ID NO: 47), T209 (SEQ ID NO: 48), T210 (SEQ ID NO: 49), T211 (SEQ ID NO: 50). In another embodiment, polypeptide comprises sequence T148 (SEQ ID NO:35). In another embodiment, the polypeptide comprises sequence T151 (SEQ ID NO:36). In another embodiment, the polypeptide comprises sequence T153 (SEQ ID NO:37). In another embodiment, the polypeptide comprises sequence T183 (SEQ ID NO:38). In another embodiment, the polypeptide comprises sequence T186 (SEQ ID NO:39). In another embodiment, the polypeptide comprises sequence T191 (SEQ ID NO:40). In another embodiment, the polypeptide comprises sequence T202 (SEQ ID NO:41). In another embodiment, the polypeptide comprises sequence T203 (SEQ ID NO:42). In another embodiment, the polypeptide comprises sequence T204 (SEQ ID NO:43). In another embodiment, the polypeptide comprises sequence T205 (SEQ ID NO:44). In another embodiment, the polypeptide comprises sequence T206 (SEQ ID NO:45). In another embodiment, the polypeptide comprises sequence T207 (SEQ ID NO:46). In another embodiment, the polypeptide comprises sequence T208 (SEQ ID NO:47). In another embodiment, the polypeptide comprises sequence T209 (SEQ ID NO:48). In another embodiment, the polypeptide comprises sequence T210 (SEQ ID NO:49). In another embodiment, the polypeptide comprises sequence T211 (SEQ ID NO:50).

Also provided herein are proteins comprising two polypeptide chains, each polypeptide chain comprising a portion of an antibody constant region and a single-chain TRAIL trimer, wherein the protein retains at least 10% of initial activity after incubation in 90% mouse serum at a final concentration of 1 μM for 7 days at 37° C. In one embodiment the TRAIL activity is measured by the EC50 of HCT116 cell killing.

In one embodiment, the TRAIL trimer comprises a set of three human TRAIL monomer moieties. In another embodiment, the polypeptide chain comprises an amino acid sequence at least 95% identical to amino acid residues 95-281, 114-281, or 120-281 of SEQ ID NO:28, and comprises a substitution at one or more of positions 121, 130, 228, and 247. In another embodiment, the polypeptide chain comprises an amino acid sequence at least 96%, 97%, 98%, or 99% identical to amino acid residues 95-281, 114-281, or 120-281 of SEQ ID NO:28.

In another embodiment, the polypeptide chain comprises a sequence selected from the group consisting of T148 (SEQ ID NO: 35), T151 (SEQ ID NO: 36), T153 (SEQ ID NO: 37), T183 (SEQ ID NO: 38), T186 (SEQ ID NO: 39), T191 (SEQ ID NO: 40), T202 (SEQ ID NO: 41), T203 (SEQ ID NO: 42), T204 (SEQ ID NO: 43), T205 (SEQ ID NO: 44), T206 (SEQ ID NO: 45), T207 (SEQ ID NO: 46), T208 (SEQ ID NO: 47), T209 (SEQ ID NO: 48), T210 (SEQ ID NO: 49), T211 (SEQ ID NO: 50).

Also provided herein are proteins comprising two polypeptide chains, each polypeptide chain comprising a portion of an antibody constant region and a single-chain TRAIL trimer, wherein the protein has a terminal half-life in mouse circulation of 10 hours or greater. In one embodiment, the TRAIL trimer comprises a set of three human TRAIL monomer moieties. In another embodiment, the polypeptide chain comprises an amino acid sequence at least 95% identical to amino acid residues 95-281, 114-281, or 120-281 of SEQ ID NO:28, and comprises a substitution at one or more of positions 121, 130, 228, and 247. In another embodiment, the polypeptide chain comprises an amino acid sequence at least 96%, 97%, 98%, or 99% identical to amino acid residues 95-281, 114-281, or 120-281 of SEQ ID NO:28.

In another embodiment, the polypeptide chain comprises a sequence selected from the group consisting of T148 (SEQ ID NO: 35), T151 (SEQ ID NO: 36), T153 (SEQ ID NO: 37), T183 (SEQ ID NO: 38), T186 (SEQ ID NO: 39), T191 (SEQ ID NO: 40), T202 (SEQ ID NO: 41), T203 (SEQ ID NO: 42), T204 (SEQ ID NO: 43), T205 (SEQ ID NO: 44), T206 (SEQ ID NO: 45), T207 (SEQ ID NO: 46), T208 (SEQ ID NO: 47), T209 (SEQ ID NO: 48), T210 (SEQ ID NO: 49), T211 (SEQ ID NO: 50).

Also provided herein, are polypeptides comprising the heavy chain of MOC31 IgG (anti-EpCAM) fused to scTRAIL. In one embodiment, the polypeptide comprises an amino acid sequence that is at least 95% identical to amino acid residues 95-281, 114-281, or 120-281 of SEQ ID NO: 28. In one embodiment, the polypeptide comprises a substitution at one or more of position 121, 130, 228, and 247 of SEQ ID: 28. In another embodiment, the polypeptide comprises at least one substitution selected from the group consisting of R121I, R130G, N228S, and I247V of SEQ ID NO: 28. In another embodiment, the polypeptide comprises at least one substitution selected from the group consisting of I247G, I247A, I247V, and I 247L. In another embodiment, the polypeptide comprises a substitution at one or both of positions 213 and 215 of SEQ ID: 28. In another embodiment, the polypeptide comprises at least one substitution selected from the group consisting of Y213W and S215D. In another embodiment, the polypeptide comprises a set of substitutions selected from the group consisting of: (i) R121I and I247V; (ii) N228S and I247V; (iii) R130G and I247V; (iv) R121I, R130G, Y213W, S215D and I247V; (v) R130G, Y213W, S215D and I247V; and (vi) R130G, Y213W, S215D, N228S and I247V. In another embodiment, the polypeptide comprises a set of substitutions selected from the group consisting of: (i) R121I, R130G, and I247V; (ii) R130G, N228S, and I247V; (iii) R121I, R130G, N228S, and I247V; (iv) R121I, N228S, and I247V; (v) R121I and R130G; (vi) R121I, R130G, and N228S; (vii) R121I and N228S; and (viii) R130G and N228S. In another embodiment, the polypeptide comprises SEQ ID NO: 99.

### DETAILED DESCRIPTION

Provided herein are TRAIL fusion polypeptides. In one aspect, single mutant polypeptide chains of an Fc-TRAIL fusion polypeptide comprised of two polypeptide chains dimerized by at least one inter-Fc disulfide bond are disclosed. In another aspect, TRAIL fusions to antibody FAB fragments or to other proteins such as albumin, e.g. human serum albumin (HSA), are provided. In yet another aspect, mutations within the TRAIL monomer that provide improved characteristics (such as thermostability and manufacturability) are provided. Also provided herein are methods of treating a cancer in a human patient by administering to the patient an effective amount of the Fc-TRAIL fusion polypeptide described herein.

### Definitions

For convenience, the meaning of certain terms and phrases used in the specification, examples, and claims, are provided below.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a”, “an”, and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

The term “about” as used herein when referring to a measurable value such as an amount, a temporal duration and the like, is encompasses variations of up to ±10% from the specified value. Unless otherwise indicated, all numbers expressing quantities of ingredients, properties such as molecular weight, reaction conditions, etc., used herein are to be understood as being modified by the term “about”.

As used herein, the term “subject” or “patient” is a human patient (e.g., a patient having cancer).

The terms “treat,” “treating,” and “treatment,” as used herein, refer to therapeutic or preventative measures described herein. The methods of “treatment” employ administration to a subject, the combination disclosed herein in order to prevent, cure, delay, reduce the severity of, or ameliorate one or more symptoms of the disease or disorder or recurring disease or disorder, or in order to prolong the survival of a subject beyond that expected in the absence of such treatment.

As used herein, “antineoplastic agent” refers to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “TRAIL” (also referred to as “Apo2L/TRAIL”, “TNF-related apoptosis-inducing ligand” and “CD253”) refers to member of the TNF family that binds and activates the death receptors (specifically DR4 and DR5). Human TRAIL amino acid sequence (1-281) (NP_003801.1) is:

(SEQ ID NO: 28)
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG.

TRAIL also binds non-signaling decoy receptors, DcR1, DcR2, and osteoprotegrin (OPG, also known as osteoclastogenesis inhibitory factor (OCIF)). TRAIL naturally occurs as a type 2 transmembrane protein, with an extracellular domain that can be cleaved to release a soluble trimeric protein. Clustering of the receptor complex, e.g., as mediated by the trimeric structure of TRAIL, is necessary for efficient signaling and induction of apoptosis by the death receptors. Additionally, higher order oligomerization of receptor complexes can amplify signaling, resulting in greater induction of apoptosis.

Beneficial mutations in TRAIL monomer provided herein for use in a single chain TRAIL molecule include individual mutations (numbered per SEQ ID NO:28, above) as follows: R121I, R130G, Y213W, S215D, N228S and I247V. Combinations of mutations are also provided. In one embodiment, the TRAIL fusion polypeptide is an Fc TRAIL fusion polypeptide. In another embodiment the TRAIL fusion polypeptide is a Fab-TRAIL fusion polypeptide. In yet another embodiment the TRAIL fusion polypeptide is an HSA-TRAIL fusion polypeptide. Suitable human serum albumin (HSA) moieties for use in such an HSA-TRAIL fusion polypeptide include native and mutant HSAs disclosed in U.S. Pat. Nos. 8,927,694 and 8,877,687.

“Peptide” or “polypeptide” refers to any peptide comprising two or more amino acids joined by peptide bonds or modified peptide bonds (e.g., peptide isosteres). Peptides can contain amino acids other than the 20 naturally occurring nucleic acid encoded amino acids, and include amino acid sequences modified either by natural processes, such as post-translational processing, or by chemical modification techniques which are well known in the art. Modifications can occur anywhere in a peptide, including the peptide backbone, the amino acid side-chains and the amino or carboxyl termini. It will be appreciated that the same type of modification can be present in the same or varying degrees at several sites in a given peptide. Also, a given polypeptide can contain many types of modifications. Polypeptides can be branched as a result of ubiquitination, and they can be cyclic, with or without branching. Cyclic, branched and branched cyclic polypeptides can result from natural posttranslational processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “variant” as used herein is defined as a modified or altered form of a wildtype sequence, e.g. where one or more amino acids may be replaced by other amino acid(s) or non-amino acid(s) which do not substantially affect function. In some embodiments, the variant may contain an altered side chain for at least one amino acid residue.

The term “antigen” as used herein is defined as an entity which elicits an immune system response. The term herein may be abbreviated to “Ag.”

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

The term “inhibit” or “inhibition” means to reduce by a measurable amount.

“Inhibitors” and “antagonists,” or “activators” and “agonists,” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity.

An “agonist” is a compound that interacts with a target to cause or promote an increase in the activation of the target (e.g., a polypeptide which agonizes (promotes) TRAIL signaling).

An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although aspects of the present invention have been specifically disclosed by various embodiments which may include preferred embodiments, exemplary embodiments and optional features, modifications and variations of the concepts herein disclosed may be resorted to by those skilled in the art. Such modifications and variations are considered to be within the scope of embodiments of the invention as described and as may be defined by the appended claims.

A. TRAIL Moieties

Provided herein are TRAIL polypeptides which comprise a TRAIL moiety. In one embodiment, the TRAIL moiety comprises one TRAIL domain (monomer). In another embodiment, the TRAIL moiety comprises two TRAIL monomers (dimer). In another embodiment, the moiety comprises three TRAIL monomers (trimer). In another embodiment, the moiety comprises the amino acid residues 95-281, 114-281, or 120-281 of SEQ ID NO:28. In another embodiment, the polypeptide comprises a TRAIL moiety linked (e.g., fused) to an antibody Fc region or a fragment thereof and/or a Fab or fragment thereof and/or an antibody and/or an albumin (e.g., HSA).

In another embodiment, the TRAIL monomer comprises full-length human TRAIL (i.e., amino acid residues 1-281 of SEQ ID NO: 28). In another embodiment, the TRAIL monomer comprises a portion of the amino acid sequence set forth in SEQ ID NO: 28. In another embodiment, the TRAIL monomer comprises amino acids 114-281 of SEQ ID NO: 28. In another embodiment, the TRAIL monomer consists of amino acids 114-281 of SEQ ID NO: 28. In another embodiment, the TRAIL domain comprises amino acid residues 95-281 of SEQ ID NO: 28. In another embodiment, the TRAIL monomer consists of amino acid residues 95-281 of SEQ ID NO: 28. In another embodiment, the TRAIL monomer comprises amino acid residues 120-281 of SEQ ID NO: 28. In another embodiment, the TRAIL monomer consists of amino acid residues 120-281 of SEQ ID NO: 28.

In another embodiment, the TRAIL domain consists of or comprises amino acid residues 90-281 of SEQ ID NO: 28. In another embodiment, the TRAIL domain consists of or comprises amino acid residues 91-281 of SEQ ID NO: 28. In another embodiment, the TRAIL domain consists of or comprises amino acid residues 92-281 of SEQ ID NO: 28. In another embodiment, the TRAIL domain consists of or comprises amino acid residues 93-281 of SEQ ID NO: 28. In another embodiment, the TRAIL domain consists of or comprises amino acid residues 94-281 of SEQ ID NO: 28. In another embodiment, the TRAIL domain consists of or comprises amino acid residues 95-281 of SEQ ID NO: 28. In another embodiment, the TRAIL domain consists of or comprises amino acid residues 96-281 of SEQ ID NO: 28. In another embodiment, the TRAIL domain consists of or comprises amino acid residues 97-281 of SEQ ID NO: 28. In another embodiment, the TRAIL domain consists of or comprises amino acid residues 98-281 of SEQ ID NO: 28. In another embodiment, the TRAIL domain consists of or comprises amino acid residues 99-281 of SEQ ID NO: 28. In another embodiment, the TRAIL domain consists of or comprises amino acid residues 100-281 of SEQ ID NO: 28. In another embodiment, the TRAIL domain consists of or comprises amino acid residues 101-281 of SEQ ID NO: 28. In another embodiment, the TRAIL domain consists of or comprises amino acid residues 102-281 of SEQ ID NO: 28. In another embodiment, the TRAIL domain consists of or comprises amino acid residues 103-281 of SEQ ID NO: 28. In another embodiment, the TRAIL domain consists of or comprises amino acid residues 104-281 of SEQ ID NO: 28. In another embodiment, the TRAIL domain consists of or comprises amino acid residues 105-281 of SEQ ID NO: 28. In another embodiment, the TRAIL domain consists of or comprises amino acid residues 106-281 of SEQ ID NO: 28. In another embodiment, the TRAIL domain consists of or comprises amino acid residues 107-281 of SEQ ID NO: 28. In another embodiment, the TRAIL domain consists of or comprises amino acid residues 108-281 of SEQ ID NO: 28. In another embodiment, the TRAIL domain consists of or comprises amino acid residues 109-281 of SEQ ID NO: 28. In another embodiment, the TRAIL domain consists of or comprises amino acid residues 110-281 of SEQ ID NO: 28. In another embodiment, the TRAIL domain consists of or comprises amino acid residues 111-281 of SEQ ID NO: 28. In another embodiment, the TRAIL domain consists of or comprises amino acid residues 112-281 of SEQ ID NO: 28. In another embodiment, the TRAIL domain consists of or comprises amino acid residues 113-281 of SEQ ID NO: 28. In another embodiment, the TRAIL domain consists of or comprises amino acid residues 114-281 of SEQ ID NO: 28. In another embodiment, the TRAIL domain consists of or comprises amino acid residues 115-281 of SEQ ID NO: 28. In another embodiment, the TRAIL domain consists of or comprises amino acid residues 116-281 of SEQ ID NO: 28. In another embodiment, the TRAIL domain consists of or comprises amino acid residues 117-281 of SEQ ID NO: 28. In another embodiment, the TRAIL domain consists of or comprises amino acid residues 118-281 of SEQ ID NO: 28. In another embodiment, the TRAIL domain consists of or comprises amino acid residues 119-281 of SEQ ID NO: 28. In another embodiment, the TRAIL domain consists of or comprises amino acid residues 120-281 of SEQ ID NO: 28. In another embodiment, the TRAIL domain consists of or comprises amino acid residues 121-281 of SEQ ID NO: 28. In another embodiment, the TRAIL domain consists of or comprises amino acid residues 122-281 of SEQ ID NO: 28. In another embodiment, the TRAIL domain consists of or comprises amino acid residues 123-281 of SEQ ID NO: 28. In another embodiment, the TRAIL domain consists of or comprises amino acid residues 124-281 of SEQ ID NO: 28. In another embodiment, the TRAIL domain consists of or comprises amino acid residues 125-281 of SEQ ID NO: 28. In another embodiment, the TRAIL domain consists of or comprises amino acid residues 126-281 of SEQ ID NO: 28. In another embodiment, the TRAIL domain consists of or comprises amino acid residues 127-281 of SEQ ID NO: 28. In another embodiment, the TRAIL domain consists of or comprises amino acid residues 128-281 of SEQ ID NO: 28. In another embodiment, the TRAIL domain consists of or comprises amino acid residues 129-281 of SEQ ID NO: 28. In another embodiment, the TRAIL domain consists of or comprises amino acid residues 130-281 of SEQ ID NO: 28.

In another embodiment, the TRAIL monomer comprises or consists of a sequence at least 80%, 85%, 90%, 91%, 92%, 93%, 94%, 95%, 96%, 97%, 98%, or 99% identical to a sequence having an N-terminus at any one of amino acid residues 90-130 of SEQ ID NO: 28 and a C terminus at any one of amino acid residues 251-281 of SEQ ID NO: 28.

In another embodiment, the TRAIL monomer comprises no more than about 250 amino acid residues, preferably no more than about 200 amino acid residues, and more preferably no more than about 150 amino acid residues. In another embodiment, the TRAIL monomer consists of no more than about 250 amino acid residues, preferably no more than about 200 amino acid residues, and more preferably no more than about 150 amino acid residues.

In another embodiment, the fusion polypeptide comprises a set of three human TRAIL monomers to form a single-chain TRAIL trimer. In one embodiment, the single-chain TRAIL trimer comprises, in amino- to carboxyl-terminal order, a first TRAIL monomer, a linker, a second TRAIL monomer, a second linker, and a third TRAIL monomer. In another embodiment, each linker consists of 15-20 amino acids. In another embodiment, each of the two inter-TRAIL monomer linkers comprises 3 G₄5 domains.

In one embodiment, the TRAIL fusion polypeptide is an Fc TRAIL fusion polypeptide. In another embodiment the TRAIL fusion polypeptide is a Fab-TRAIL fusion polypeptide. In yet another embodiment the TRAIL fusion polypeptide is an HSA-TRAIL fusion polypeptide. Suitable human serum albumin (HSA) moieties for use in such an HSA-TRAIL fusion polypeptide include native and mutant HSAs disclosed in U.S. Pat. Nos. 8,927,694 and 8,877,687.

In one embodiment, the TRAIL moiety binds to at least one of its signaling receptors (specifically DR4 and DR5) or non-signaling decoy receptors, DcR1, DcR2, and osteoprotegrin (OPG). In another embodiment, the TRAIL moiety induces apoptosis.

B. TRAIL Mutations

Provided herein are TRAIL monomer, dimer, timers, and fusion polypeptides thereof comprising an amino acid substitution at one or more of positions 121, 130, 228, and 247 of SEQ ID NO: 28. Beneficial mutations in TRAIL monomer provided herein for use in a single chain TRAIL molecule include individual mutations (numbered per SEQ ID NO:28, above) as follows: R121I, R130G, Y213W, S215D, N228S and I247V. Combinations of mutations are also provided, including numbered combinations 1)-6) as follows: 1) R121I and I247V; 2) N228S and I247V; 3) R130G and I247V; 4) R121I, R130G, Y213W, S215D and I247V; 5) R130G, Y213W, S215D and I247V; 6) R130G, Y213W, S215D, N228S and I247V. Combinations of mutations may also include numbered combinations 1)-8) as follows: (1) R121I, R130G, and I247V; (2) R130G, N228S, and I247V; (3) R121I, R130G, N228S, and I247V; (4) R121I, N228S, and I247V; (5) R121I and R130G; (6) R121I, R130G, and N228S; (7) R121I and N228S; and (8) R130G and N228S. Specific TRAIL mutants comprising each of the foregoing numbered combinations of mutations are set forth in the Examples and Figures below as: combination 1) “T148”, combination 2) “T151”, combination 3) “T153”, combination 4) “T183”, combination 5) “T186” and combination 6) “T191”. Other TRAIL mutants useful in the compositions and methods provided herein include “T182”, “T196”, “T202”, “T203”, “T204”, “T205”, “T206”, “T207”, “T208”, “T209”, “T210”, and “T211”.

In one embodiment, the TRAIL monomer comprises the amino acid sequence of SEQ ID NO: 82 or a portion thereof. In another embodiment, the TRAIL monomer consists of the amino acid sequence of SEQ ID NO: 82. In another embodiment, the TRAIL monomer comprises the amino acid sequence of SEQ ID NO: 83 or a portion thereof. In another embodiment, the TRAIL monomer consists of the amino acid sequence of SEQ ID NO: 83. In another embodiment, the TRAIL monomer comprises the amino acid sequence of SEQ ID NO: 84 or a portion thereof. In another embodiment, the TRAIL monomer consists of the amino acid sequence of SEQ ID NO: 84. In another embodiment, the TRAIL monomer comprises the amino acid sequence of SEQ ID NO: 85 or a portion thereof. In another embodiment, the TRAIL monomer consists of the amino acid sequence of SEQ ID NO: 85. In another embodiment, the TRAIL monomer comprises the amino acid sequence of SEQ ID NO: 86 or a portion thereof. In another embodiment, the TRAIL monomer consists of the amino acid sequence of SEQ ID NO: 86. In another embodiment, the TRAIL monomer comprises the amino acid sequence of SEQ ID NO: 87 or a portion thereof. In another embodiment, the TRAIL monomer consists of the amino acid sequence of SEQ ID NO: 87. In another embodiment, the TRAIL monomer comprises the amino acid sequence of SEQ ID NO: 88 or a portion thereof. In another embodiment, the TRAIL monomer consists of the amino acid sequence of SEQ ID NO: 88. In another embodiment, the TRAIL monomer comprises the amino acid sequence of SEQ ID NO: 89 or a portion thereof. In another embodiment, the TRAIL monomer consists of the amino acid sequence of SEQ ID NO: 89. In another embodiment, the TRAIL monomer comprises the amino acid sequence of SEQ ID NO: 90 or a portion thereof. In another embodiment, the TRAIL monomer consists of the amino acid sequence of SEQ ID NO: 90. In another embodiment, the TRAIL monomer comprises the amino acid sequence of SEQ ID NO: 91 or a portion thereof. In another embodiment, the TRAIL monomer consists of the amino acid sequence of SEQ ID NO: 91. In another embodiment, the TRAIL monomer comprises the amino acid sequence of SEQ ID NO: 92 or a portion thereof. In another embodiment, the TRAIL monomer consists of the amino acid sequence of SEQ ID NO: 92. In another embodiment, the TRAIL monomer comprises the amino acid sequence of SEQ ID NO: 93 or a portion thereof. In another embodiment, the TRAIL monomer consists of the amino acid sequence of SEQ ID NO: 93. In another embodiment, the TRAIL monomer comprises the amino acid sequence of SEQ ID NO: 94 or a portion thereof. In another embodiment, the TRAIL monomer consists of the amino acid sequence of SEQ ID NO: 94. In another embodiment, the TRAIL monomer comprises the amino acid sequence of SEQ ID NO: 95 or a portion thereof. In another embodiment, the TRAIL monomer consists of the amino acid sequence of SEQ ID NO: 95. In another embodiment, the TRAIL monomer comprises the amino acid sequence of SEQ ID NO: 96 or a portion thereof. In another embodiment, the TRAIL monomer consists of the amino acid sequence of SEQ ID NO: 96. In another embodiment, the TRAIL monomer comprises the amino acid sequence of SEQ ID NO: 97 or a portion thereof. In another embodiment, the TRAIL monomer consists of the amino acid sequence of SEQ ID NO: 97. In another embodiment, the TRAIL monomer comprises the amino acid sequence of SEQ ID NO: 104 or a portion thereof. In another embodiment, the TRAIL monomer consists of the amino acid sequence of SEQ ID NO: 104. In another embodiment, the TRAIL monomer comprises the amino acid sequence of SEQ ID NO: 105 or a portion thereof. In another embodiment, the TRAIL monomer consists of the amino acid sequence of SEQ ID NO: 105.

In another embodiment, the TRAIL monomer comprises an amino acid sequence that is highly identical to any one of the sequences set forth herein. For example, in one embodiment, the TRAIL monomer comprises an amino acid sequences at least 80%, 85%, 90%, 91%, 92%, 93%, 94%, 95%, 96%, 97%, 98%, or 99% identical to amino acid residues 1-254 of SEQ ID NO: 4. In another embodiment, the TRAIL monomer comprises an amino acid sequences at least 80%, 85%, 90%, 91%, 92%, 93%, 94%, 95%, 96%, 97%, 98%, or 99% identical to amino acid residues 95-281, 114-281, or 120-281 of SEQ ID NO: 28. In another embodiment, the TRAIL monomer consists of amino acid sequences at least 80%, 85%, 90%, 91%, 92%, 93%, 94%, 95%, 96%, 97%, 98%, or 99% identical to amino acid residues 1-281, 95-281, 114-281, or 120-281 of SEQ ID NO: 28. In another embodiment, the TRAIL monomer comprises an amino acid sequences at least 80%, 85%, 90%, 91%, 92%, 93%, 94%, 95%, 96%, 97%, 98%, or 99% identical to SEQ ID NO: 82-97, 104, and 105. In another embodiment, the TRAIL monomer consists of an amino acid sequence at least 80%, 85%, 90%, 91%, 92%, 93%, 94%, 95%, 96%, 97%, 98%, or 99% identical to SEQ ID NO: 82-97, 104, and 105. In another embodiment, TRAIL monomer comprises an amino acid sequence at least 95% identical to residues 1-281, 95-281, 114-281, or 120-281 of SEQ ID NO: 28. In a particular embodiment, the TRAIL monomer comprises an amino acid sequence at least 95% identical to SEQ ID NO: 82-97, 104, and 105.

“% identical” refers to two or more nucleic acid or polypeptide sequences or subsequences that are the same (100% identical) or have a specified percentage of nucleotide or amino acid residues that are the same, when the two sequences are aligned for maximum correspondence and compared. To align for maximum correspondence, gaps may be introduced into one of the sequences being compared. The amino acid residues or nucleotides at corresponding positions are then compared and quantified. When a position in the first sequence is occupied by the same residue as the corresponding position in the second sequence, then the sequences are identical at that position. The percent identity between the two sequences is a function of the number of identical positions shared by the sequences (e.g., % identity=#of identical positions/total #of positions (e.g., overlapping positions)×100). In certain embodiments, the two sequences are the same length. The determination that one sequence is a measured % identical with another sequence can be determined using a mathematical algorithm. A non-limiting example of a mathematical algorithm utilized for such comparison of two sequences is incorporated in the ALIGN program (version 2.0) which is part of the GCG sequence alignment software package. When utilizing the ALIGN program e.g., for comparing amino acid sequences, a PAM120 weight residue table, a gap length penalty of 12, and a gap penalty of 4 may be used. Additional algorithms for sequence analysis are well known in the art and many are available online.

While exemplified in the foregoing mutant single chain TRAIL polypeptides, these mutations and combinations are contemplated as being present in any single chain TRAIL construct, regardless of precise format or fusion partner (if any), for example, in single chain TRAIL constructs comprising three TRAIL monomers, wherein the each mutation, or combination of mutations can be independently present or absent from each of the three monomers.

In one embodiment, the mutant TRAIL fusion polypeptide is an Fc-TRAIL fusion polypeptide. In another embodiment the mutant TRAIL fusion polypeptide is a Fab-TRAIL fusion polypeptide. In another embodiment the mutant TRAIL fusion polypeptide is a Fab-Fc-TRAIL fusion polypeptide. In yet another embodiment the mutant TRAIL fusion polypeptide is an HSA-TRAIL fusion polypeptide. Suitable human serum albumin (HSA) moieties for use in such an HSA-TRAIL fusion polypeptide include native and mutant HSAs disclosed in U.S. Pat. Nos. 8,927,694 and 8,877,687.

C. Exemplary TRAIL Fusion Polypeptides

i. TRAIL Monomers, Dimers, and Trimers

As provided herein, TRAIL polypeptides may be TRAIL monomers, dimers, or trimers in a single polypeptide chain construct, regardless of precise format or fusion partner (if any). For example, a single chain TRAIL construct can comprise one, two, or three TRAIL monomers.

Each monomer may contain a mutation or combination of mutations can be independently present or absent from each of the three monomers. The TRAIL mutations may be selected from amino acid substitution at one or more of positions 121, 130, 213, 215, 228, and 247 of SEQ ID NO: 28. Beneficial mutations in TRAIL monomer provided herein for use in a single chain TRAIL molecule include individual mutations (numbered per SEQ ID NO:28, above) as follows: R121I, R130G, Y213W, S215D, N228S and I247V.

In one aspect each of the three monomers contains the same mutation or the same combination of mutations, in another aspect two of the three monomers contains the same mutation or the same combination of mutations, while the third comprises a different mutation or combination of mutations, or no mutation, and in yet another aspect, each of the three monomers comprises a different mutation or combination of mutations, or no mutation is present in one or two of the three monomers. For example, exemplary single chain mutant TRAIL trimers may be selected from “T148”, “T151”, “T153”, “t182”, “T183”, “T186”, “T191”, “T196”, “T202”, “T203”, “T204”, “T205”, “T206”, “T207”, “T208”, “T209”, “T210”, and “T211” (SEQ ID NO: 61-81, 102, and 103).

ii. Fc-TRAIL Fusion Polypeptides

In one embodiment, a TRAIL moiety is linked to an Fc region or fragment thereof.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (C_{H2}) and third (C_{H3}) constant domains of the antibody's two heavy chains; IgM and IgE Fc regions comprise three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

In another embodiment, the Fc-TRAIL fusion polypeptide comprises a native sequence Fc region. A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1:1).

In certain embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity.

For example, one may make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fe. Such Fc region variants will generally comprise at least one amino acid modification in the Fc region. Combining amino acid modifications is thought to be particularly desirable. For example, the variant Fc region may include two, three, four, five, etc substitutions therein, e.g. of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKE sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase antibody dependent cellular cytotoxicity (ADCC) and/or to increase the affinity for an Fey receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439, Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/3247, and 267E/268F/324T. Other modifications for enhancing FcyR and complement interactions include but are not limited to substitutions 298A, 333A, 334A, 326A, 2471, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Other Fc modifications that can be made to Fcs are those for reducing or ablating binding to FcγR and/or complement proteins, thereby reducing or ablating Fe-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 2368, 237K, 267R, 269R, 325L, and 328R, wherein numbering is according to the EU index. An Fc variant may comprise 236R/328R. Other modifications for reducing FcyR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCT Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor FcγRIIb may also be used. Such variants may provide an Fc fusion protein with immunomodulatory activities related to FcyRIIb⁺ cells, including for example B cells and monocytes. In one embodiment, the Fc variants provide selectively enhanced affinity to FcyRIIb relative to one or more activating receptors. Modifications for altering binding to FcyRIIb include one or more modifications at a position selected from the group consisting of 234, 235, 236, 237, 239, 266, 267, 268, 325, 326, 327, 328, and 332, according to the EU index. Exemplary substitutions for enhancing FcyRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 2361), 2391), 266M, 267E, 268D, 268E, 328F, 328W, and 328Y. Other Fc variants for enhancing binding to FcyRIIb include 235Y/267E. 236D/267E, 239D/268D, 239D/267E, 267E/268D, 267E/268E, and 267E/328F.

The affinities and binding properties of an Fc region for its ligand may be determined by a variety of in vitro assay methods biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In certain embodiments, the antibody is modified to increase its biological half-life. Various approaches are possible. For example, this may be done by increasing the binding affinity of the Fc region for FcRn. For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 621.3-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In certain embodiments, hybrid IgG isotypes with particular biological characteristics may be used. For example, an IgG1/IgG3 hybrid variant may be constructed by substituting IgG1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed that comprises one or more substitutions, e.g., one or more of the following amino acid substitutions: 233E, 234L, 235L, −236G (referring to an insertion of a glycine at position 236), and 327A.

Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

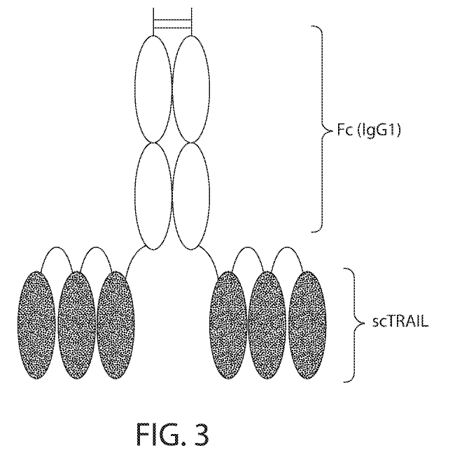

In another embodiment, an Fc-TRAIL polypeptide chain is dimerized to a second Fc-TRAIL polypeptide chain (see FIG. 3). In a particular embodiment, the two Fc-TRAIL polypeptide chains are dimerized by at least one inter-Fc disulfide bond. In another embodiment, the two Fc-TRAIL polypeptide chains are dimerized by at least two inter-Fc disulfide bonds. In another embodiment, the two Fc-TRAIL polypeptide chains are dimerized by at least three inter-Fc disulfide bonds.

In a particular embodiment, the Fc-TRAIL fusion polypeptide comprises two polypeptide chains dimerized by at least one inter-Fc disulfide bond, each chain comprising a human IgG Fc moiety peptide-bound to a set of three human 4-TRAIL domains to form a single unbranched polypeptide comprising, in amino- to carboxyl-terminal order, the Fc moiety, a linker, a first TRAIL monomer, a inter-monomer linker, a second TRAIL monomer, a second inter-monomern linker, and a third TRAIL monomer, wherein each linker consists of 15-20 amino acids and each of the two inter-TRAIL monomer linkers comprises 3 G₄S motifs.

In another embodiment, the Fc region is modified with respect to effector function, so as to enhance the effectiveness of the polypeptide in treating a disease, e.g., cancer. For example cysteine residue(s) may be introduced in the Fc region, thereby allowing inter-chain disulfide bond formation in this region. The homodimeric polypeptide thus generated may have improved internalization capability and/or increased complement-mediated cell killing and antibody-dependent cellular cytotoxicity (ADCC). Homodimeric polypeptides with enhanced anti-tumor activity may also be prepared using heterobifunctional cross-linkers. Alternatively, a polypeptide can be engineered which has dual Fc regions and may thereby have enhanced complement lysis and ADCC capabilities.

In a particular embodiment, the Fc-TRAIL fusion polypeptide comprises a human IgG Fc moiety, or fragment thereof, bound to a set of three human TRAIL domains to form a single unbranched polypeptide comprising, in amino- to carboxyl-terminal order, the Fc moiety, a linker, a first TRAIL monomer, an inter-monomer linker, a second TRAIL monomer, a second inter-monomer linker, and a third TRAIL monomer. In a particular embodiment, for example, the Fc-TRAIL fusion polypeptide comprises any one of SEQ ID NO: 35-50, 100, and 101. In another embodiment, the Fc-TRAIL fusion polypeptide comprises at least one, two, three, or four mutations not found in native wild-type human TRAIL.

In one embodiment, the Fc-TRAIL fusion polypeptide induces cancer cell apoptosis.

iii. Fab-Fc-TRAIL and Fab-TRAIL Fusion Polypeptides

The Fc-TRAIL fusion polypeptides described herein may further comprise an antibody Fab region, or fragment thereof (e.g., Fab-Fc-TRAIL fusion polypeptide). “Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge. In another embodiment, the TRAIL fusion comprises a full-length heavy and light chain, or fragment thereof. In another embodiment the TRAIL fusion comprises a full-length antibody.

In one embodiment, the Fab-Fc-TRAIL fusion or the full-length heavy and light chain heavy chain TRAIL fusion, or fragment thereof, can be dimerized to a second fusion polypeptide chain. In a particular embodiment, the two fusion polypeptide chains are dimerized by at least one inter-Fc disulfide bond. In another embodiment, the two fusion polypeptide chains are dimerized by at least two inter-Fc disulfide bonds. In another embodiment, the two fusion polypeptide chains are dimerized by at least three inter-Fc disulfide bonds.

In another embodiment the Fab-Fc, heavy and light chain, full-length antibody, or fragment thereof, is fused to a TRAIL moiety with a linker. In another embodiment the linker is an amino acid linker. Modifications can also be made within one or more of the framework or joining regions of the heavy and/or the light chain variable regions of the Fab region or antibody, so long as antigen binding affinity subsequent to these modifications is maintained.

In another embodiment, the Fab-Fc-TRAIL fusion polypeptide comprises a human Fab moiety, or fragment thereof, bound to a human Fc moiety, or fragment thereof, bound to a set of three human TRAIL monomers to form a single unbranched polypeptide comprising, in amino- to carboxyl-terminal order, the Fc moiety, a linker, a first TRAIL monomer, an inter-monomer linker, a second TRAIL monomer, a second inter-monomer linker, and a third TRAIL monomer. In another embodiment, the Fab-Fc-TRAIL fusion polypeptide comprises at least one, two, three, or four mutations not found in native wild-type human TRAIL.

The TRAIL fusions describe herein, may also comprise an antibody Fab region, or antigen-binding portion thereof (Fab-TRAIL). In one embodiment the Fab region comprises a full-length heavy chain. In another embodiment, the Fab region comprises a full-length heavy and light chain, or fragment thereof. In another embodiment, the Fab-TRAIL fusion, can be dimerized to a second fusion polypeptide chain. In a particular embodiment, the two fusion polypeptide chains are dimerized by at least one inter-Fc disulfide bond. In another embodiment, the two fusion polypeptide chains are dimerized by at least two inter-Fc disulfide bonds. In another embodiment, the two fusion polypeptide chains are dimerized by at least three inter-Fc disulfide bonds.

In another embodiment the Fab, or fragment thereof, is fused to a TRAIL moiety with a linker. In another embodiment the linker is an amino acid linker. Modifications can also be made within one or more of the framework or joining regions of the heavy and/or the light chain variable regions of the Fab region or antibody, so long as antigen binding affinity subsequent to these modifications is maintained.

In another embodiment, the Fab-TRAIL fusion polypeptide comprises a human Fab moiety, or fragment thereof, bound to a set of three human TRAIL monomers to form a single unbranched polypeptide comprising, in amino- to carboxyl-terminal order, the Fab moiety, a linker, a first TRAIL monomer, an inter-monomer linker, a second TRAIL monomer, a second inter-monomer linker, and a third TRAIL monomer. In another embodiment, the Fab-TRAIL fusion polypeptide comprises at least one, two, three, or four mutations not found in native wild-type human TRAIL. An exemplary Fab-TRAIL fusion polypeptide may comprise an anti-EpCAM Fab fused to a soluble TRAIL (scTRAIL) moiety (e.g., SEQ ID NO: 99).

iv. Albumin-TRAIL Fusion Polypeptides

In another embodiment, a TRAIL moiety is linked to an albumin moiety (e.g., Human Serum Albumin (HSA)). In another embodiment, the albumin-TRAIL fusion polypeptide comprises one, two, or three TRAIL monomers.

In a particular embodiment, a single TRAIL fusion polypeptide chain comprises a human serum albumin moiety peptide-bound to a set of three human TRAIL monomers to form a single unbranched polypeptide comprising, in amino- to carboxyl-terminal order, the albumin moiety, a linker, a first TRAIL monomer, an inter-monomer linker, a second TRAIL monomer, a second inter-monomer linker, and a third TRAIL monomer.

v. Bispecific Fusion Polypeptides

Also provided are bispecific antibody fusions. In one embodiment, the TRAIL moiety is fused to the c-terminus of a heavy chain of a bispecific antibody. Bispecific antibodies herein include at least two binding specificities for the same or different proteins which preferably bind non-overlapping or non-competing epitopes. Such bispecific antibodies can include additional binding specificities, e.g., a third protein binding specificity for another antigen, such as the product of an oncogene. Bispecific antibodies can be prepared as full length antibodies or antibody fragments (e.g. F(ab′)₂ bispecific antibodies).

D. Methods for Producing Fusion Polypeptides

The TRAIL fusion proteins described herein can be produced by standard recombinant techniques. Methods for recombinant production are widely known in the state of the art and comprise protein expression in prokaryotic and eukaryotic cells with subsequent isolation of the antibody and usually purification to a pharmaceutically acceptable purity. For the expression of the binding proteins in a host cell, nucleic acids encoding the respective polypeptides are inserted into expression vectors by standard methods. Expression is performed in appropriate prokaryotic or eukaryotic host cells (such as CHO cells, NSO cells, SP2/0 cells, HEK293 cells, COS cells, PER.C6 cells, yeast, or E. coli cells), and the binding protein is recovered from the cells (supernatant or cells after lysis). General methods for recombinant production of antibodies are well-known in the state of the art and described, for example, in the review articles of Makrides, S. C., Protein Expr. Purif 17 183-202 (1999); Geisse, S., et al, Protein Expr. Purif. 8 271-282 (1996); Kaufman, R. J., MoI. Biotechnol. 16 151-161 (2000); Werner, R. G., Drug Res. 48 870-880 (1998).

The polypeptides may be suitably separated from the culture medium by conventional purification procedures. Purification can be performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxylmethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A. Appl. Biochem. Biotech. 75 93-102 (1998)). DNA and RNA encoding the polypeptides are readily isolated and sequenced using conventional procedures.

E. Linkers

A variety of linkers can be used in the fusion polypeptides described herein. “Linked to” refers to direct or indirect linkage or connection of, in context, amino acids or nucleotides. “Linker” refers to one or more amino acids connecting two domains or regions together. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Additional linkers suitable for use can be found in the Registry of Standard Biological Parts at http://partsregistry.org/Protein_domains/Linker (see also, e.g., Crasto C J and Feng J A. LINKER: a program to generate linker sequences for fusion proteins. Protein Eng 2000 May; 13(5) 309-12 and George R A and Heringa J. An analysis of protein domain linkers: their classification and role in protein folding. Protein Eng 2002 November; 15(11) 871-9). A linker may be 1-10, 10-20, 20-30, 30-40, 40-50, 50-60, 60-70, 70-80, 80-90 or at least 90-100 amino acids long.

An Fc region or albumin can be separated from the TRAIL moiety by a linker. Additionally, each TRAIL monomer of the TRAIL moiety can be separated by an inter-monomer linker. In certain embodiments, each linker or inter-domain linker comprises 5-25 amino acids. In one embodiment, the linker or inter-domain linker comprises 5-10, 5-15, 5-20, 5-25, 10-15, 10-20, 10-25, 15-20, 15-25, or 20-25 amino acids. In another embodiment, the linker or inter-monomer linker comprises 5, 6, 7, 8, 9, 10, 11, 12, 13, 14, 15, 16, 17, 18, 19, 20, 21, 22, 23, 24, or 25 amino acids. In a particular embodiment, the linker or inter-monomer linker comprises 15-20 amino acids. In another embodiment, the linker or inter-monomer linker comprises at least one, two, or three G₄S motifs. A G₄S motif comprises four glycine residues followed by one serine residue (i.e., amino acid sequence GGGGS). In particular embodiments, the linker or inter-monomer linker comprises three G₄S motifs.

F. Compositions

In another aspect, compositions comprising the polypeptides described herein are provided, as well as methods of using such compositions for diagnostic purposes or to treat a disease in a patient. The compositions provided herein contain one or more of the polypeptides disclosed herein, formulated together with a carrier (e.g., a “pharmaceutically acceptable carrier”). In one embodiment, the composition comprises a polypeptide comprising a TRAIL moiety linked (e.g., fused) to an antibody Fc region or a fragment thereof and/or a Fab or fragment thereof and/or an antibody and/or an albumin (e.g., HSA).

As used herein, “pharmaceutically acceptable carrier” includes any and all solvents, dispersion media, coatings, antibacterial and antifungal agents, isotonic and absorption delaying agents, and the like that are physiologically compatible. The carrier can be a solvent or dispersion medium containing, for example, water, ethanol, polyol (for example, glycerol, propylene glycol, and liquid polyethylene glycol, and the like), and suitable mixtures thereof. Saline solutions and aqueous dextrose and glycerol solutions can be employed as liquid carriers, particularly for injectable solutions. Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any excipient, diluent or agent is incompatible with the active compound, use thereof in the pharmaceutical compositions provided herein is contemplated. Supplementary active compounds (e.g., additional anti-cancer agents) can also be incorporated into the compositions.

Therapeutic compositions typically must be sterile and stable under the conditions of manufacture and storage. The composition can be formulated as a solution, microemulsion, liposome, or other ordered structure suitable to high drug concentration. The composition, if desired, can also contain minor amounts of wetting or solubility enhancing agents, stabilizers, preservatives, or pH buffering agents. In many cases, it will be useful to include isotonic agents, for example, sodium chloride, sugars, polyalcohols such as mannitol, sorbitol, glycerol, propylene glycol, and liquid polyethylene glycol in the composition. Prolonged absorption of the injectable compositions can be brought about by including in the composition an agent that delays absorption, for example, monostearate salts and gelatin

In the context of treating a disease in a patient, preferably, the carrier is suitable for intravenous, intramuscular, subcutaneous, parenteral, spinal or epidermal administration (e.g., by injection or infusion). Depending on the route of administration, the polypeptide may be coated in a material to protect them from the action of acids and other natural conditions that may inactivate proteins. For example, the polypeptide may be administered to a patient in an appropriate carrier, for example, in liposomes, or a diluent. Pharmaceutically acceptable diluents include saline and aqueous buffer solutions. Liposomes include water-in-oil-in-water CGF emulsions, as well as conventional liposomes. The composition can be administered by a variety of methods known in the art. As will be appreciated by the skilled artisan, the route and/or mode of administration will vary depending upon the desired results.

Pharmaceutical compositions may be administered alone or in combination therapy, i.e., combined with other agents (e.g., as discussed in further detail below).

G. Methods and Uses

The polypeptides, compositions, and methods described herein have numerous in vitro and in vivo utilities involving, for example, inducing cancer cell apoptosis and/or enhancement of immune response. For example, the polypeptides described herein (e.g., a polypeptide comprising a TRAIL moiety linked (e.g., fused) to an antibody Fc region or a fragment thereof and/or a Fab or fragment thereof and/or an antibody and/or an albumin (e.g., HSA)) can be administered to cells in culture, in vitro or ex vivo, or to human subjects, e.g., in vivo, to induce cancer cell apoptosis and/or enhance immunity in a variety of diseases.

The terms “treat,” “treating,” and “treatment,” as used herein, refer to therapeutic or preventative measures described herein. The methods of “treatment” employ administration to a patient the polypeptides disclosed herein in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” refers to the amount of a therapy which is sufficient to reduce or ameliorate the severity and/or duration of a disease or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another therapy (e.g., prophylactic or therapeutic agent).

In one embodiment, the disease is cancer. The term “cancer” as used herein is defined as a tissue of uncontrolled growth or proliferation of cells, such as a tumor. As used herein, the term includes pre-malignant as well as malignant cancers.

Further provided are methods for inhibiting growth of tumor cells in a subject comprising administering to the subject the polypeptides described herein, such that growth of the tumor is inhibited in the subject. As used herein, the term “inhibits growth” of a tumor includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

Cancers can be cancers with solid tumors or blood malignancies (liquid tumors). The methods described herein may also be used for treatment of metastatic cancers, unresectable and/or refractory cancers (e.g., cancers refractory to previous immunotherapy), and recurrent cancers.

Also, provided herein are methods of modifying an immune response in a subject comprising administering to the subject the polypeptides described herein, such that the immune response in the subject is modified. Preferably, the response is enhanced, stimulated or up-regulated. In one embodiment, methods of stimulating (activating) immune cells for cancer therapy by administering the polypeptides described herein to a patient (e.g., a human patient) are provided. In another embodiment, methods of maintaining T cells for adoptive cell transfer therapy are provided. In another embodiment, methods of stimulating proliferation of T cells for adoptive cell transfer therapy are provided. T cells that can be enhanced stimulated with the polypeptides described herein include CD4+ T cells and CD8+ T cells. The T cells can be T_{eff} cells, e.g., CD4+T_{eff} cells, CD8+T_{eff} cells, Thelper (Tₕ) cells and T cytotoxic (T_{c}) cells.

H. Kits and Articles of Manufacture

Further provided are kits containing the polypeptide compositions described herein and instructions for use. Kits typically include a packaged combination of reagents in predetermined amounts with instructions and a label indicating the intended use of the contents of the kit. The term label or instruction includes any writing, or recorded material supplied on or with the kit, or which otherwise accompanies the kit at any time during its manufacture, transport, sale or use. It can be in the form prescribed by a governmental agency regulating the manufacture, use or sale of pharmaceuticals or biological products, which notice reflects approval by the agency of the manufacture, use or sale for administration to a human or for veterinary use. The label or instruction can also encompass advertising leaflets and brochures, packaging materials, and audio or video instructions.

For example, in some embodiments, the kit contains the polypeptide in suitable containers and instructions for administration in accordance with the treatment regimens described herein. In some embodiments, the kit further comprises an additional antineoplastic agent. In some embodiments, the polypeptides are provided in suitable containers as a dosage unit for administration. Suitable containers include, for example, bottles, vials, syringes, and test tubes. The containers may be formed from a variety of materials such as glass or plastic.

In some embodiments, the polypeptides are provided in lyophilized form, and the kit may optionally contain a sterile and physiologically acceptable reconstitution medium such as water, saline, buffered saline, and the like. It may further include other materials desirable from a commercial and user standpoint, including other buffers, diluents, filters, needles, syringes, and package inserts with instructions for use, for example, comprising administration schedules, to allow a practitioner (e.g., a physician, nurse, or patient) to administer the composition contained therein.

All references cited throughout this application, for example patent documents including issued or granted patents or equivalents; patent application publications; and non-patent literature documents or other source material; are hereby incorporated by reference herein in their entireties, as though individually incorporated by reference. Any sequence listing and sequence listing information is considered part of the disclosure herewith.

Those skilled in the art will recognize, or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

The following examples are merely illustrative and should not be construed as limiting the scope of this disclosure in any way as many variations and equivalents will become apparent to those skilled in the art upon reading the present disclosure.

All patents, patent applications and publications cited herein are incorporated herein by reference in their entireties.

### EXAMPLES

### Example 1: Developing an Improved Sctrail Format Methods

### Protein Expression

The nucleotide sequence encoding TRAIL is codon optimized for HEK-293 (ATCC CRL-1573) expression and the following sequences T1-T9 (SEQ ID NO:1-SEQ ID NO:9) are synthesized and cloned into plasmid pCEP4 (Invitrogen) at the KpnI and NotI restriction sites. The underlined text denotes the leader sequence and the heavy chain Fv of anti-EpCAM antibody MOC-31 is shown in bold. The leader sequence of each is underlined.

T1
(SEQ ID NO: 1)
MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSTSEETISTVQEKQQNISPLVRERGPQRVAAHITGTRGRSNTL
SSPNSKNEKALGRKINSWESSRSGHSFLSNLHLRNGELVIHEKGFYYIYS
QTYFRFQEEIKENTKNDKQMVQYIYKYTSYPDPILLMKSARNSCWSKDAE
YGLYSIYQGGIFELKENDRIFVSVTNEHLIDMDHEASFFGAFLVGGGGGS
TSEETISTVQEKQQNISPLVRERGPQRVAAHITGTRGRSNTLSSPNSKNE
KALGRKINSWESSRSGHSFLSNLHLRNGELVIHEKGFYYIYSQTYFRFQE
EIKENTKNDKQMVQYIYKYTSYPDPILLMKSARNSCWSKDAEYGLYSIYQ
GGIFELKENDRIFVSVTNEHLIDMDHEASFFGAFLVGGGGGSTSEETIST
VQEKQQNISPLVRERGPQRVAAHITGTRGRSNTLSSPNSKNEKALGRKIN
SWESSRSGHSFLSNLHLRNGELVIHEKGFYYIYSQTYFRFQEEIKENTKN
DKQMVQYIYKYTSYPDPILLMKSARNSCWSKDAEYGLYSIYQGGIFELKE
NDRIFVSVTNEHLIDMDHEASFFGAFLVG
T2

(SEQ ID NO: 2)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSTSEETISTVQEKQQNISPLVRERGPQRVAAHITGTRGRSNTL
SSPNSKNEKALGRKINSWESSRSGHSFLSNLHLRNGELVIHEKGFYYIYS
QTYFRFQEEIKENTKNDKQMVQYIYKYTSYPDPILLMKSARNSCWSKDAE
YGLYSIYQGGIFELKENDRIFVSVTNEHLIDMDHEASFFGAFLVGGGGGS
GGGGSTSEETISTVQEKQQNISPLVRERGPQRVAAHITGTRGRSNTLSSP
NSKNEKALGRKINSWESSRSGHSFLSNLHLRNGELVIHEKGFYYIYSQTY
FRFQEEIKENTKNDKQMVQYIYKYTSYPDPILLMKSARNSCWSKDAEYGL
YSIYQGGIFELKENDRIFVSVTNEHLIDMDHEASFFGAFLVGGGGGSGGG
GSTSEETISTVQEKQQNISPLVRERGPQRVAAHITGTRGRSNTLSSPNSK
NEKALGRKINSWESSRSGHSFLSNLHLRNGELVIHEKGFYYIYSQTYFRF
QEEIKENTKNDKQMVQYIYKYTSYPDPILLMKSARNSCWSKDAEYGLYSI
YQGGIFELKENDRIFVSVTNEHLIDMDHEASFFGAFLVG
T3

(SEQ ID NO: 3)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSTSEETISTVQEKQQNISPLVRERGPQRVAAHITGTRGRSNTL
SSPNSKNEKALGRKINSWESSRSGHSFLSNLHLRNGELVIHEKGFYYIYS
QTYFRFQEEIKENTKNDKQMVQYIYKYTSYPDPILLMKSARNSCWSKDAE
YGLYSIYQGGIFELKENDRIFVSVTNEHLIDMDHEASFFGAFLVGGGGGS
GGGGSGGGGSTSEETISTVQEKQQNISPLVRERGPQRVAAHITGTRGRSN
TLSSPNSKNEKALGRKINSWESSRSGHSFLSNLHLRNGELVIHEKGFYYI
YSQTYFRFQEEIKENTKNDKQMVQYIYKYTSYPDPILLMKSARNSCWSKD
AEYGLYSIYQGGIFELKENDRIFVSVTNEHLIDMDHEASFFGAFLVGGGG
GSGGGGSGGGGSTSEETISTVQEKQQNISPLVRERGPQRVAAHITGTRGR
SNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWS
KDAEYGLYSIYQGGIFELKENDRIFVSVTNEHLIDMDHEASFFGAFLVG
T4

(SEQ ID NO: 4)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSVRERGPQRVAAHITGTRGRSNTLSSPNSKNEKALGRKINSWE
SSRSGHSFLSNLHLRNGELVIHEKGFYYIYSQTYFRFQEEIKENTKNDKQ
MVQYIYKYTSYPDPILLMKSARNSCWSKDAEYGLYSIYQGGIFELKENDR
IFVSVTNEHLIDMDHEASFFGAFLVGGGGGSVRERGPQRVAAHITGTRGR
SNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWS
KDAEYGLYSIYQGGIFELKENDRIFVSVTNEHLIDMDHEASFFGAFLVGG
GGGSVRERGPQRVAAHITGTRGRSNTLSSPNSKNEKALGRKINSWESSRS
GHSFLSNLHLRNGELVIHEKGFYYIYSQTYFRFQEEIKENTKNDKQMVQY
IYKYTSYPDPILLMKSARNSCWSKDAEYGLYSIYQGGIFELKENDRIFVS
VTNEHLIDMDHEASFFGAFLVG
T5

(SEQ ID NO: 5)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSVRERGPQRVAAHITGTRGRSNTLSSPNSKNEKALGRKINSWE
SSRSGHSFLSNLHLRNGELVIHEKGFYYIYSQTYFRFQEEIKENTKNDKQ
MVQYIYKYTSYPDPILLMKSARNSCWSKDAEYGLYSIYQGGIFELKENDR
IFVSVTNEHLIDMDHEASFFGAFLVGGGGGSGGGGSVRERGPQRVAAHIT
GTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSAR
NSCWSKDAEYGLYSIYQGGIFELKENDRIFVSVTNEHLIDMDHEASFFGA
FLVGGGGGSGGGGSVRERGPQRVAAHITGTRGRSNTLSSPNSKNEKALGR
KINSWESSRSGHSFLSNLHLRNGELVIHEKGFYYIYSQTYFRFQEEIKEN
TKNDKQMVQYIYKYTSYPDPILLMKSARNSCWSKDAEYGLYSIYQGGIFE
LKENDRIFVSVTNEHLIDMDHEASFFGAFLVG
T6

(SEQ ID NO: 6)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSVRERGPQRVAAHITGTRGRSNTLSSPNSKNEKALGRKINSWE
SSRSGHSFLSNLHLRNGELVIHEKGFYYIYSQTYFRFQEEIKENTKNDKQ
MVQYIYKYTSYPDPILLMKSARNSCWSKDAEYGLYSIYQGGIFELKENDR
IFVSVTNEHLIDMDHEASFFGAFLVGGGGGSGGGGSGGGGSVRERGPQRV
AAHITGTRGRSNTLSSPNSKNEKALGRKINSWESSRSGHSFLSNLHLRNG
ELVIHEKGFYYIYSQTYFRFQEEIKENTKNDKQMVQYIYKYTSYPDPILL
MKSARNSCWSKDAEYGLYSIYQGGIFELKENDRIFVSVTNEHLIDMDHEA
SFFGAFLVGGGGGSGGGGSGGGGSVRERGPQRVAAHITGTRGRSNTLSSP
NSKNEKALGRKINSWESSRSGHSFLSNLHLRNGELVIHEKGFYYIYSQTY
FRFQEEIKENTKNDKQMVQYIYKYTSYPDPILLMKSARNSCWSKDAEYGL
YSIYQGGIFELKENDRIFVSVTNEHLIDMDHEASFFGAFLVG
T7

(SEQ ID NO: 7)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSQRVAAHITGTRGRSNTLSSPNSKNEKALGRKINSWESSRSGH
SFLSNLHLRNGELVIHEKGFYYIYSQTYFRFQEEIKENTKNDKQMVQYIY
KYTSYPDPILLMKSARNSCWSKDAEYGLYSIYQGGIFELKENDRIFVSVT
NEHLIDMDHEASFFGAFLVGGGGGSQRVAAHITGTRGRSNTLSSPNSKNE
KALGRKINSWESSRSGHSFLSNLHLRNGELVIHEKGFYYIYSQTYFRFQE
EIKENTKNDKQMVQYIYKYTSYPDPILLMKSARNSCWSKDAEYGLYSIYQ
GGIFELKENDRIFVSVTNEHLIDMDHEASFFGAFLVGGGGGSQRVAAHIT
GTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSAR
NSCWSKDAEYGLYSIYQGGIFELKENDRIFVSVTNEHLIDMDHEASFFGA
FLVG
T8

(SEQ ID NO: 8)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSQRVAAHITGTRGRSNTLSSPNSKNEKALGRKINSWESSRSGH
SFLSNLHLRNGELVIHEKGFYYIYSQTYFRFQEEIKENTKNDKQMVQYIY
KYTSYPDPILLMKSARNSCWSKDAEYGLYSIYQGGIFELKENDRIFVSVT
NEHLIDMDHEASFFGAFLVGGGGGSGGGGSQRVAAHITGTRGRSNTLSSP
NSKNEKALGRKINSWESSRSGHSFLSNLHLRNGELVIHEKGFYYIYSQTY
FRFQEEIKENTKNDKQMVQYIYKYTSYPDPILLMKSARNSCWSKDAEYGL
YSIYQGGIFELKENDRIFVSVTNEHLIDMDHEASFFGAFLVGGGGGSGGG
GSQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYP
DPILLMKSARNSCWSKDAEYGLYSIYQGGIFELKENDRIFVSVTNEHLID
MDHEASFFGAFLVG
T9

(SEQ ID NO: 9)

MGTPAQLLFLLLLWLPDTTGQVQLQQSGPELKKPGETVKISCKASGYTFT
NYGMNWVKQAPGRGLKWMGWINTYTGESTYADDFKGRFAFSLETSASAAY
LQINNLKNEDTATYFCARFAIKGDYWGQGTTLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCAGAGGGGSGGG
GSGGGGSSQRVAAHITGTRGRSNTLSSPNSKNEKALGRKINSWESSRSGH
SFLSNLHLRNGELVIHEKGFYYIYSQTYFRFQEEIKENTKNDKQMVQYIY
KYTSYPDPILLMKSARNSCWSKDAEYGLYSIYQGGIFELKENDRIFVSVT
NEHLIDMDHEASFFGAFLVGGGGGSGGGGSGGGGSQRVAAHITGTRGRSN
TLSSPNSKNEKALGRKINSWESSRSGHSFLSNLHLRNGELVIHEKGFYYI
YSQTYFRFQEEIKENTKNDKQMVQYIYKYTSYPDPILLMKSARNSCWSKD
AEYGLYSIYQGGIFELKENDRIFVSVTNEHLIDMDHEASFFGAFLVGGGG
GSGGGGSGGGGSQRVAAHITGTRGRSNTLSSPNSKNEKALGRKINSWESS
RSGHSFLSNLHLRNGELVIHEKGFYYIYSQTYFRFQEEIKENTKNDKQMV
QYIYKYTSYPDPILLMKSARNSCWSKDAEYGLYSIYQGGIFELKENDRIF
VSVTNEHLIDMDHEASFFGAFLVG
The light chain of MOC-31 (SEQ ID NO: 10) is
also synthesized and cloned into the KpnI and
NotI sites in pCEP4.

SEQ ID NO: 10

MGTPAQLLFLLLLWLPDTTGDIVMTQSAFSNPVTLGTSASISCRSTKSLL
HSNGITYLYWYLQKPGQSPQLLIYQMSNLASGVPDRFSSSGSGTDFTLRI
SRVEAEDVGVYYCAQNLEIPRTFGGGTKLEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC

HEK-293F cells (FREESTYLE HEK-293 cells adapted for suspension culture, ThermoFisher Cat.#R79007) stably expressing the anti-apoptotic protein Bcl-XL are grown in FREESTYLE F17 media (Gibco) containing 4 mM L-glutamine (Gibco) and 1% PLURONIC F-68 (Gibco) as suspension cultures in flasks with rotation (125 rpm). Cells are separately and singly co-transfected with a mixture of 0.5 μg of one of plasmids pCEP4-T1 through pCEP4-T9, 0.5 μg of plasmid pCEP4-MOC31 light chain (1 μg of total DNA), and 2.5 μg of linear 25 kDa polyethylenimine (Polysciences Inc.) per milliliter of cell culture. Density of cells at time of transfection is 1.5-2.0 e6 cells/ml. Cells are fed the following day with Tryptone N1 (“TN1”, Organotechnie) added to a final concentration of 5 mg/ml. Six days post transfection, cell cultures are centrifuged for 15 min at 5,000×g to pellet the cells. The supernatant media are decanted from the cells and filtered using 0.2 μm filter in preparation for purification.

### Protein Purification

Media containing the anti-EpCAM Fab-scTRAIL variants are separately loaded onto MABSELECT (GE Heathcare) resin using an AKTA Explorer (Amersham Biosciences). Following affinity capture, the resin is washed with phosphate buffered saline (PBS), pH 7.4 (Gibco®) and eluted with 0.1 M glycine-HCl, pH 3.5. The acid eluate is rapidly neutralized using 1:100 volume of 1 M Tris base. Proteins are dialyzed into PBS, pH 7.4 overnight and aliquoted the next day for storage at −80° C.

### SDS-PAGE

One microgram of each of the purified anti-EpCAM Fab-scTRAIL variants is incubated in the presence or absence of 2-mercaptoethanol (1% final) for 10 minutes at 95° C. Samples are electrophoresed on NUPAGE 4-12% Bis Tris Gel (Invitrogen) and visualized using SIMPLYBLUE SAFESTAIN (Invitrogen). Stained gels are scanned using an ODYSSEY CLx imager (LI-COR Biosciences).

### Size Exclusion Chromatography

TSKGEL SuperSW3000 column (4.6 mm ID×30 cm)(Tosoh BioSciences) is equilibrated with 400 mM NaClO₄, 150 mM NaCl, pH 6.5 using a Agilent 1100 HPLC (Agilent). Fifty micrograms of protein is injected at a flow rate of 0.35 ml/min and absorbance at 280 nm is recorded over a 20 minute period.

### Cell Culture

HeLa cells are obtained from American Tissue Type Collection (ATCC) and cultured in flasks with DMEM media (Gibco) supplemented with 10% FBS, 100 units/ml penicillin, and 100 μg/ml streptomycin.

### Luminescent Cell Viability Assay

Cells are seeded at 10,000 cells per well in 96 well tissue culture plate. Twenty-four hours later cells are incubated with increasing concentrations of Fab-scTRAIL proteins. After a 24 hour treatment period, the amount of cellular ATP is detected using CELLTITER-GLO Assay (Promega) and measured on a SYNERGY H1 plate reader (BioTek). Luminescence is normalized to untreated controls and duplicates are averaged and plotted as a function of Fab-scTRAIL protein concentration. Non-linear regression is fitted using a 4 parameter least square fit using PRISM software (GraphPad).

### Results

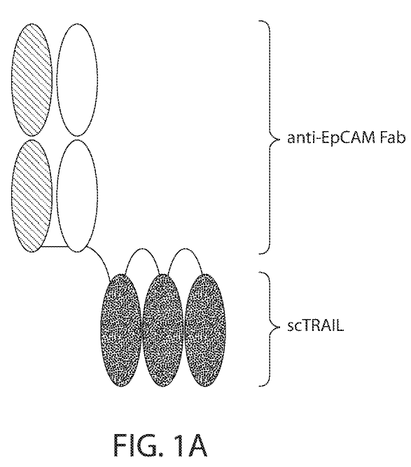

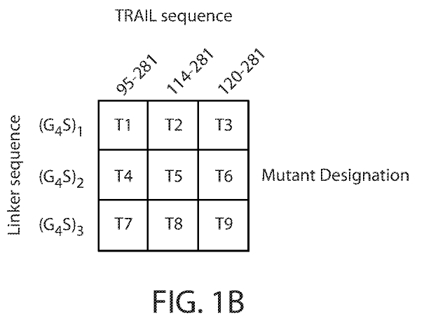

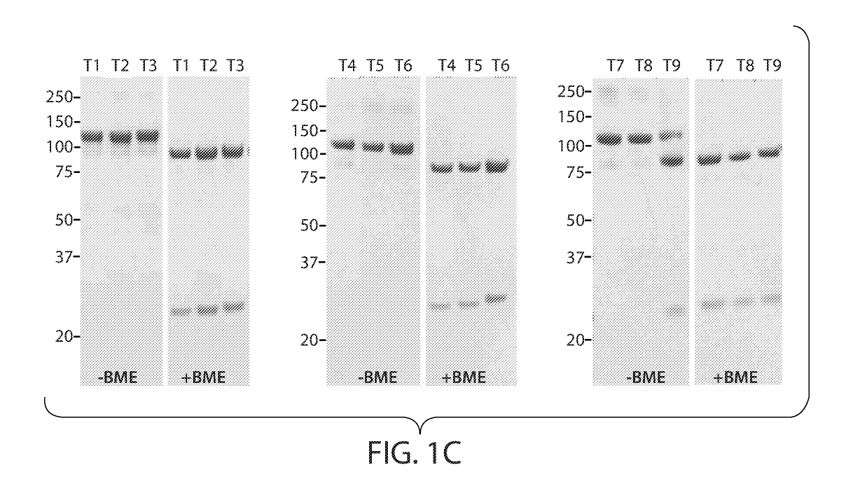

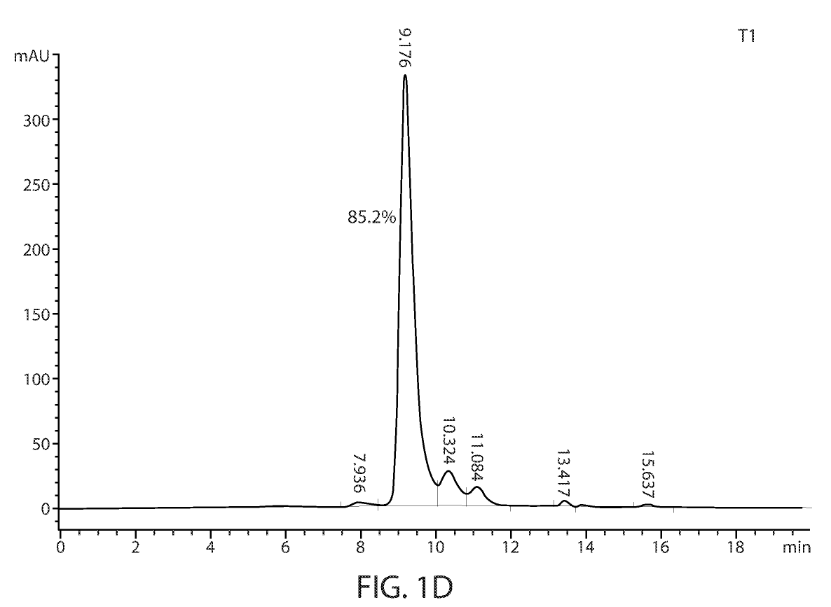

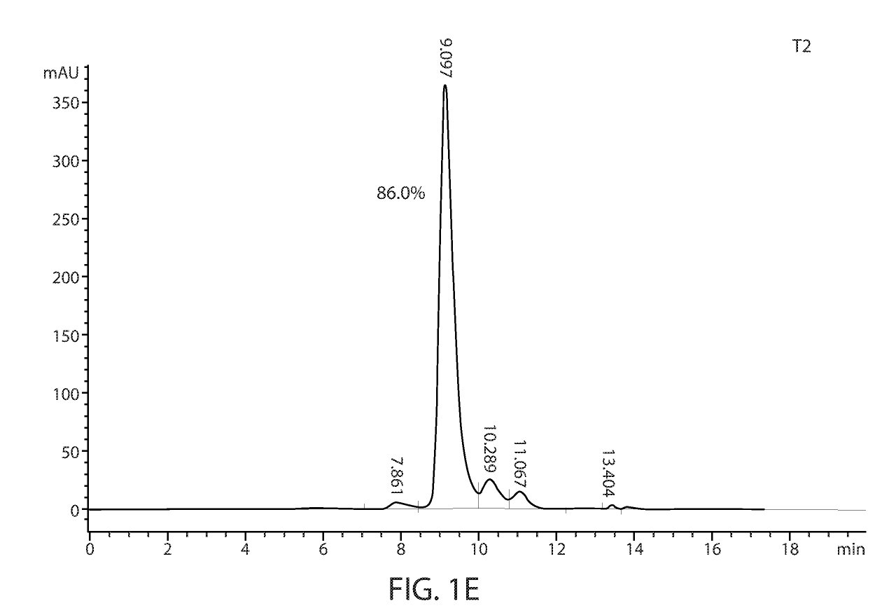

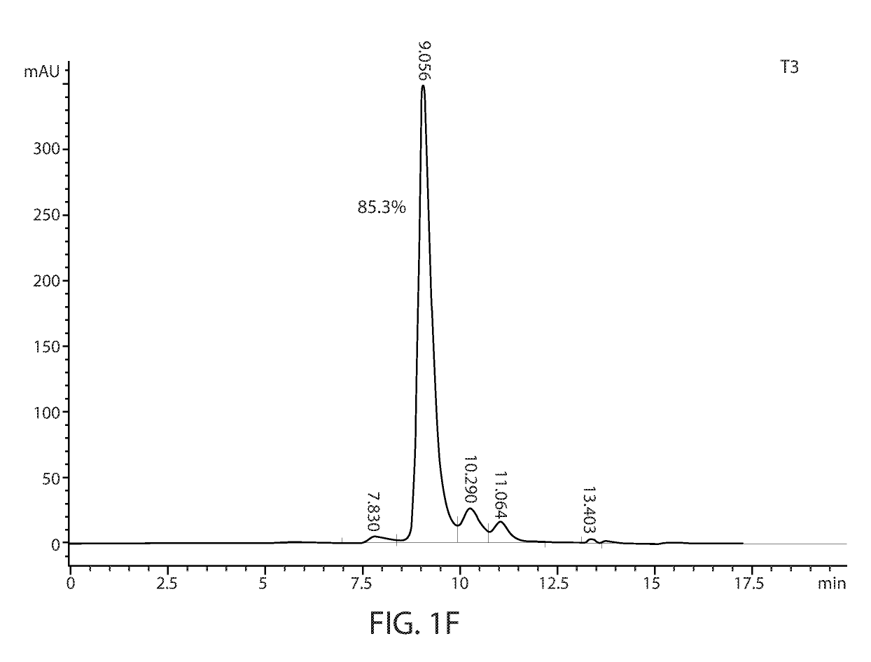

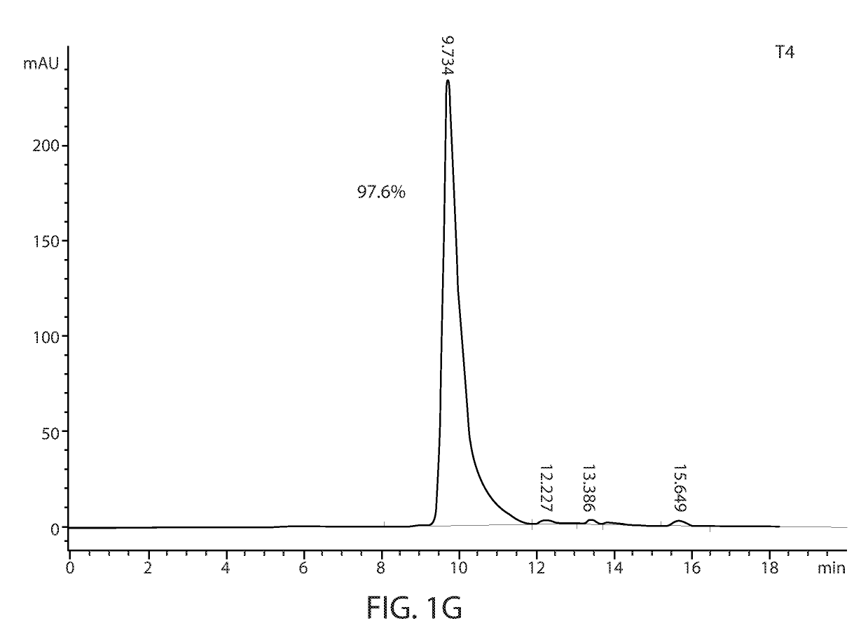

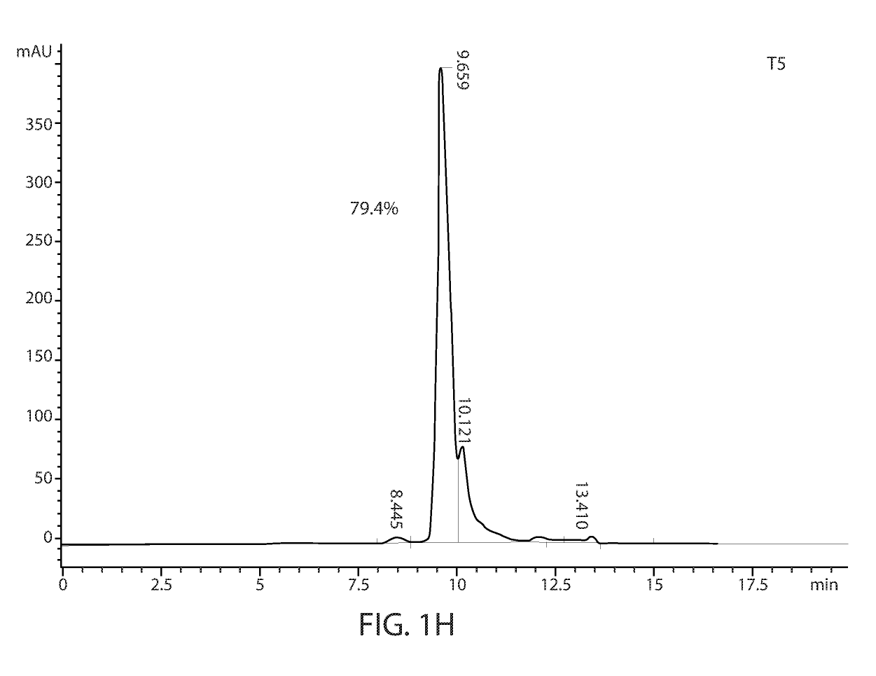

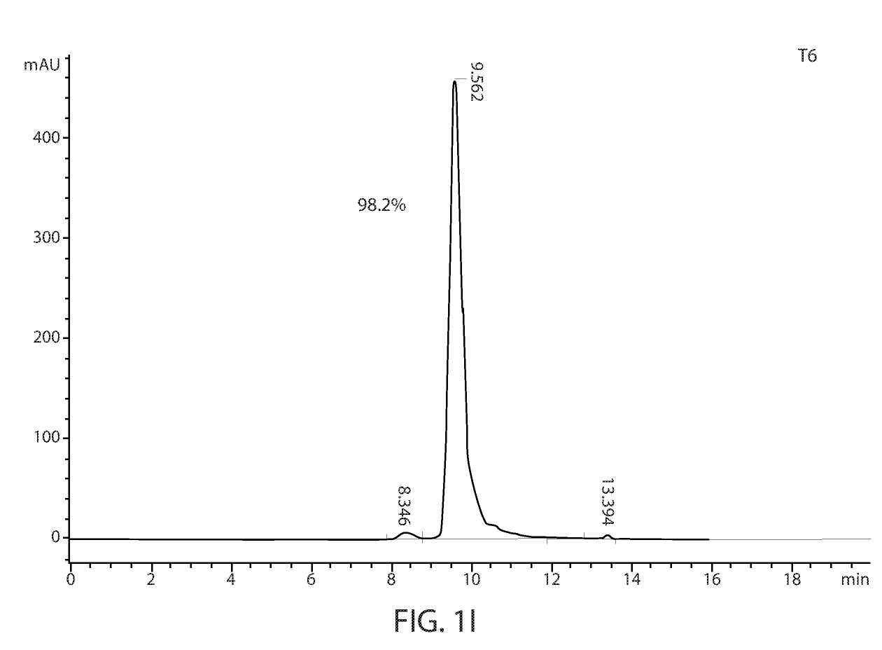

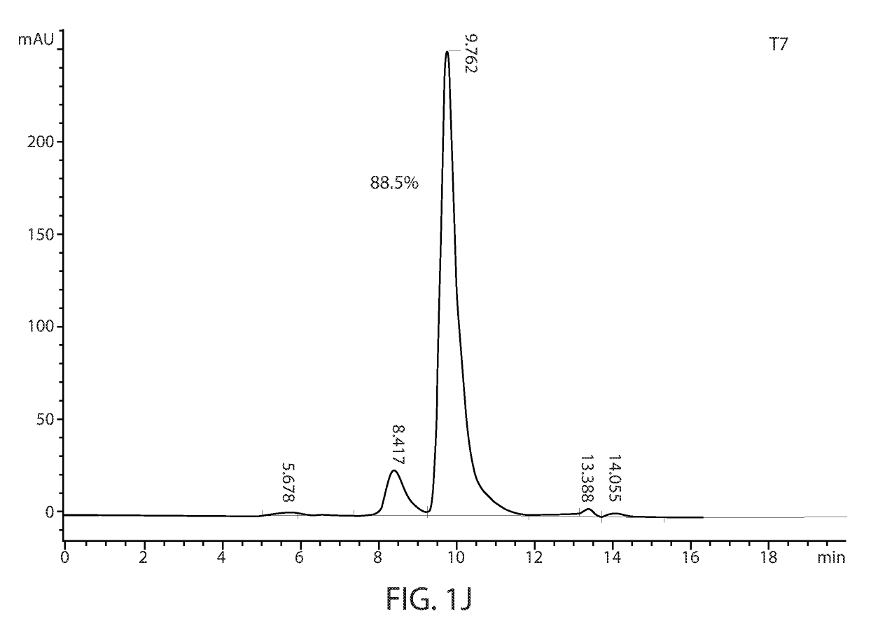

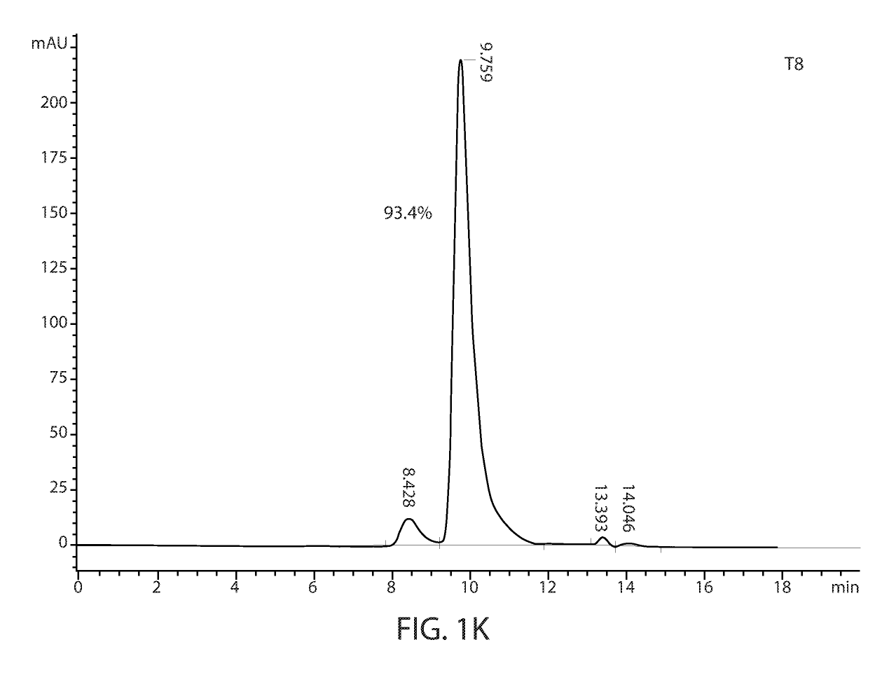

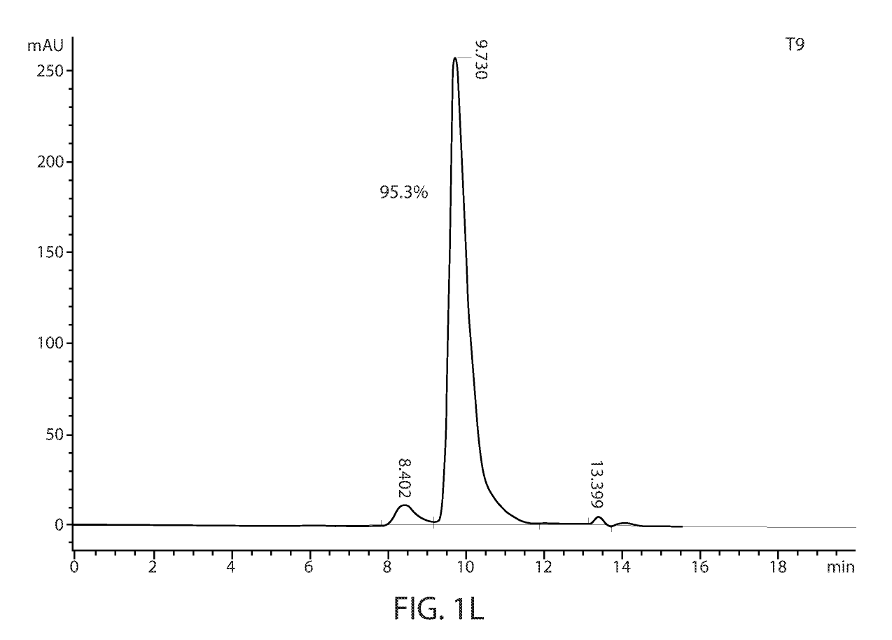

An improved single chain TRAIL (scTRAIL) fusion protein was designed. As a test fusion partner, an immunoglobulin-derived polypeptide is selected, in particular, in this Example, scTRAIL was fused to the C-terminus of the heavy chain of anti-EpCAM Fab (MOC-31) (FIG. 1A). Three different lengths of TRAIL sequences and three different lengths of glycine serine linkers for connecting the TRAIL sequences into a single linear polypeptide chain are systematically investigated (FIG. 1B).

A total of nine Fab-scTRAIL fusion variants are produced in HEK-293F cells stably overexpressing Bcl-XL and purified using protein A chromatography. As shown in FIG. 1C, for each variant the predicted observed migration of each protein under reducing and non-reducing conditions will correspond to the predicted non-reduced and reduced molecular weights (Table 2).

TABLE 2

| Fab-scTRAIL | Non reduced MW | Reduced MW |
|---|---|---|
| Variant | (kDa) | (kDa) |
| T1 | 113.6 | 90.3 |
| T2 | 114.3 | 90.9 |
| T3 | 114.9 | 91.5 |
| T4 | 107.3 | 83.9 |
| T5 | 107.9 | 84.6 |
| T6 | 108.6 | 85.2 |
| T7 | 105.2 | 81.8 |
| T8 | 105.8 | 82.5 |
| T9 | 106.5 | 83.2 |
| MOC31 LC | 23.4 | 23.4 |

The combination of short TRAIL sequence (TRAIL amino acids 120-281) and long linker length (15 amino acids: G₄S×3 (SEQ ID NO: 106)) in the T9 variant is believed to have a deleterious effect on disulfide formation between the MOC31 heavy and light chains and an ˜83 kDa band is predicted to appear in the non-reduced sample. This should not be observed for the remaining variants, thus the T9 variant is not suitable.

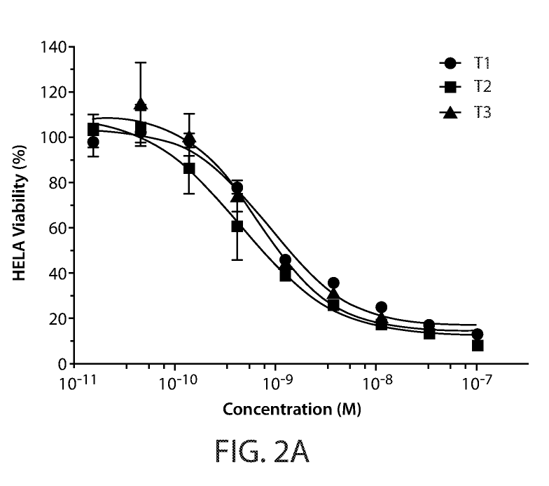

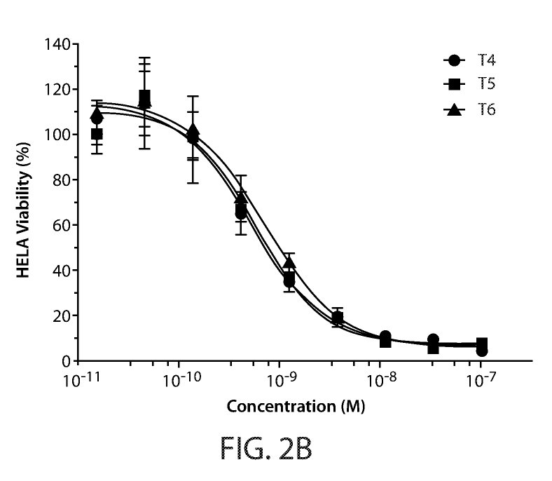

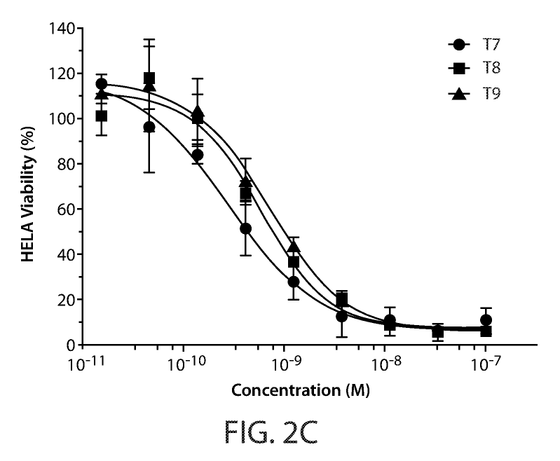

Analytical size exclusion is expected to show that among all variants, T6 contains the highest percentage of a single major species (about 98%) (FIGS. 1D-L). All variants are predicted to be functional in a cell viability assay using HELA cells (FIGS. 2A-C). Although T7 is expected to show a minor improvement in potency among all the variants (IC₅₀=2.76e-10), the TRAIL sequence and linker length of the T6 variant is selected for use because of its predicted favorable SEC profile. Next, a human IgG1 Fc as a fusion partner to the T6 variant is to be used to produce Fc-scTRAIL.

### Example 2: Expression and Purification of Fc-Sctrail

### Methods

### Protein Expression

The nucleotide sequence encoding Fc-scTRAIL was synthesized and cloned into plasmid pCEP4 (Invitrogen) at the KpnI and NotI restriction sites. In the following sequence, the leader sequence (which is removed during expression) is shown in bold while the 3 TRAIL monomers are indicated by different underlinings: position 1 ( . . . ) position 2 (_) and position 3 (=) and the Fc sequence is in italics.

SEQ ID NO: 11
MGTPAQLLFLLLLWLPDTTGEPKSSDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLS

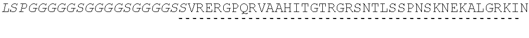

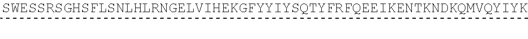

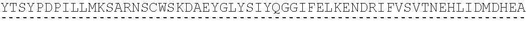

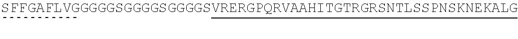

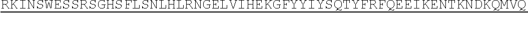

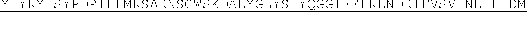

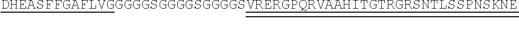

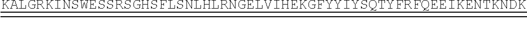

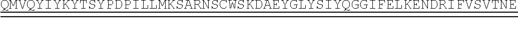

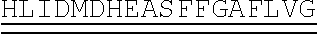

Fc-scTRAIL proteins were expressed in HEK-293F cells stably expressing Bcl-XL and purified as described in Example 1.

### SDS-PAGE and Size Exclusion Chromatography

SDS-PAGE and SEC were carried out as described in Example 1.

### Results

### Fc-scTRAIL is Well Expressed and can be Purified in Non-Aggregated Form

scTRAIL was fused to the Fc of human IgG1 (SEQ ID NO:11) to improve pharmacokinetics. An additional benefit to this format is the presence of two TRAIL cytokines in close proximity due to homodimerization of the Fc fragment (FIG. 3). This is advantageous because increased clustering of TRAIL mimics the membrane bound form of the cytokine and improves the strength of the pro apoptotic signal across many cancer cell lines.

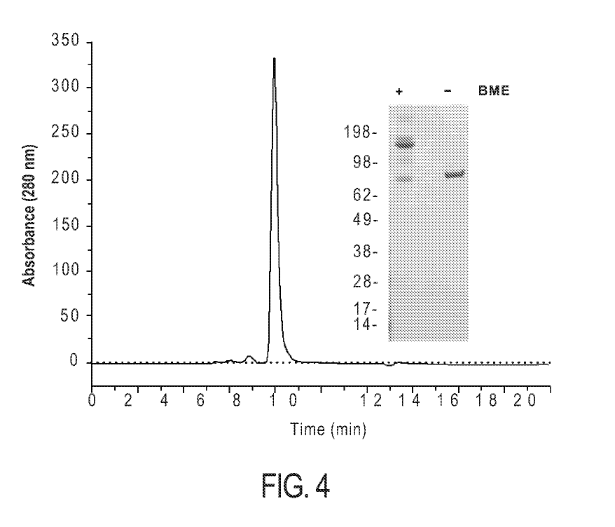

The observed molecular weight of purified Fc-scTRAIL corresponded to the predicted molecular weight of 175 and 87 kDa for disulfide linked homodimer and reduced monomer respectively (FIG. 4, gel insert). Additional bands were observed in the non-reduced sample that were not present under reducing conditions. This is believed to be due to incorrect intra-chain disulfide bond formation within the TRAIL trimer leading to an abnormal migration on the gel. Regarding the higher molecular weight species, this is believed to be due to inter-chain disulfide bond formation between two Fc-scTRAIL homodimers. In the non-reduced sample, a band was observed migrating at the same position as the reduced sample, indicating that a minor fraction of the Fc-scTRAIL homodimer is not disulfide linked. Using analytical size exclusion chromatography (FIG. 4), purified Fc-scTRAIL was observed to be a single major species (˜98%) with a retention time of 7.94 minutes, which is consistent with its theoretical molecular weight.

### Example 3: In Vitro Activity of Fc-scTRAIL

### Methods

### Cell Culture

COLO205, HCT116, DU145, PANC1, and Jurkat were cultured in flasks with RPMI 1640 media (Gibco®) supplemented with 10% FBS, 100 units/ml penicillin, and 100 μg/ml streptomycin.

### Luminescent Cell Viability Assay

This assay was carried out as described in Example 1. Antibodies were cross-linked using an equimolar concentration of anti-human Fc antibody (Jackson Immunoresearch).

### Results

Fc-scTRAIL Induces Cell Kill Across Cell Lines with Greater Potency than Agonistic Antibodies

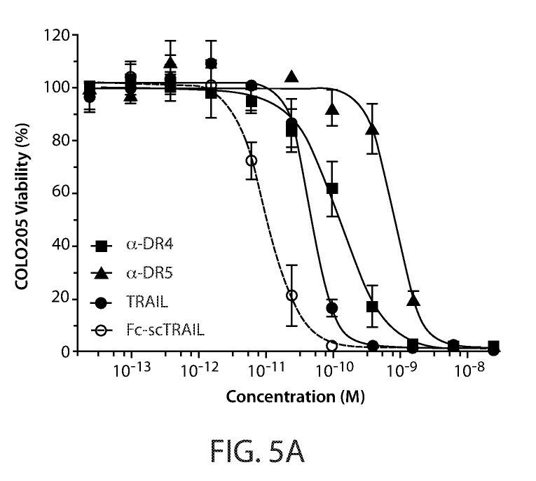

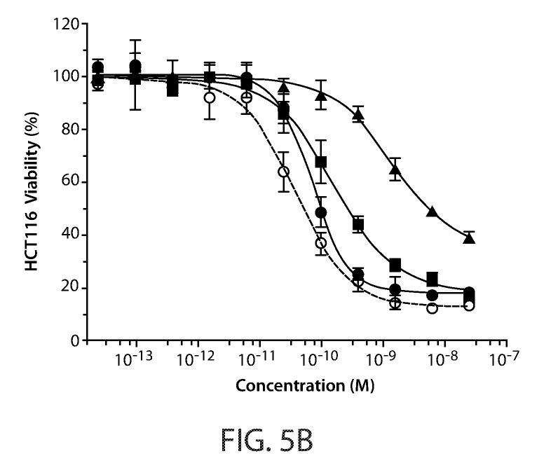

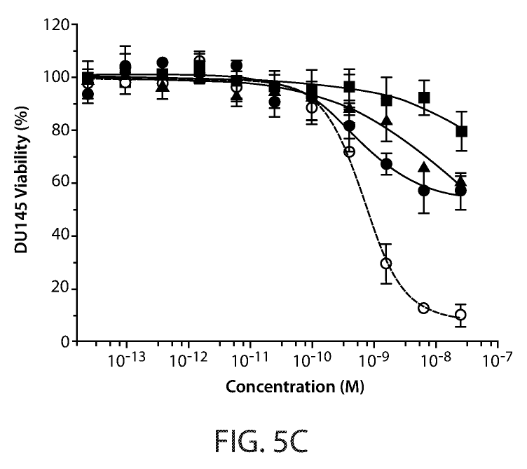

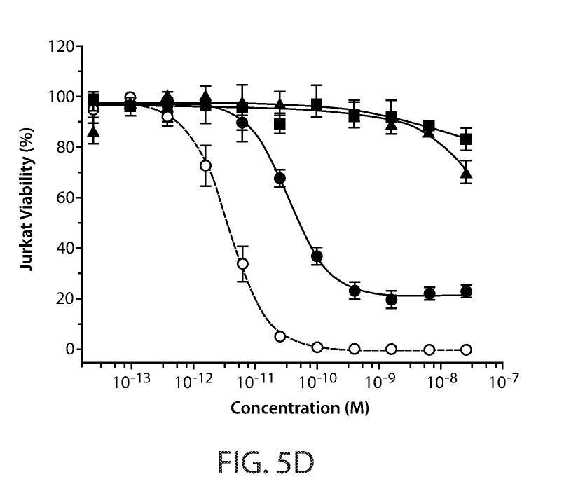

Fc-scTRAIL is functionally active as observed in a cell viability assay using the cancer cells lines COLO205 (colon), HCT116 (colon), DU145 (prostate), and Jurkat (T lymphocyte). Compared to TRAIL and agonistic DR4 (Pukac et al., Br. J. Cancer, 2005 Apr. 25; 92(8):1430-41) and DR5 (Adams et al., Cell Death Differ., 2008 April; 15(4):751-61) antibodies, Fc-scTRAIL was most active in inducing apoptosis (FIG. 5A-5D). In both COLO205 and HCT116 cells, Fc-scTRAIL induced cell death at lower concentrations as indicated by the IC₅₀s of the viability curves. In DU145 and Jurkat cells, Fc-scTRAIL induced the maximum reduction in cell viability. This improved potency supports our therapeutic design where having two TRAIL homotrimers per molecule and hexavalent binding of death receptors is better than the trivalent and bivalent receptor binding of TRAIL and agonistic antibodies, respectively.

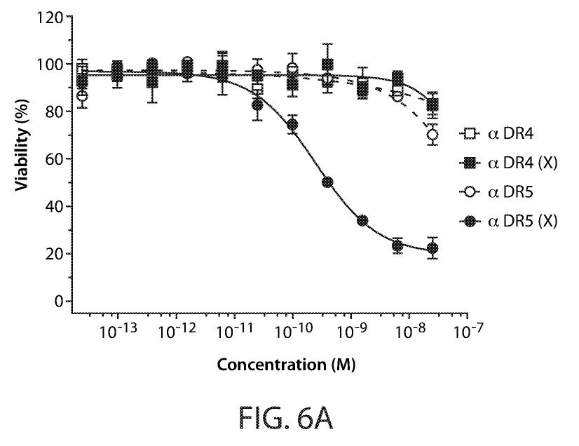

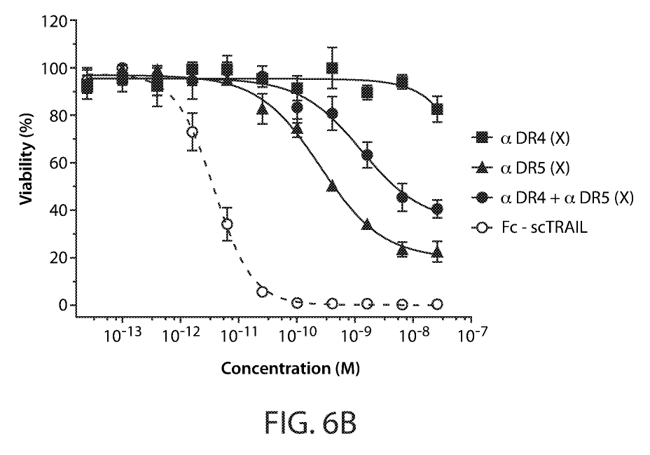

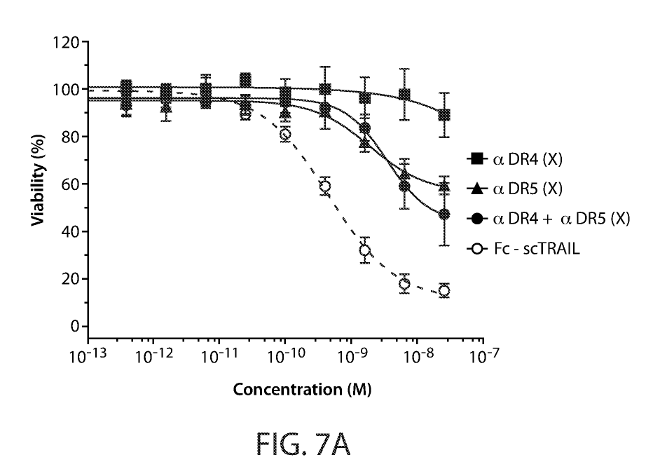

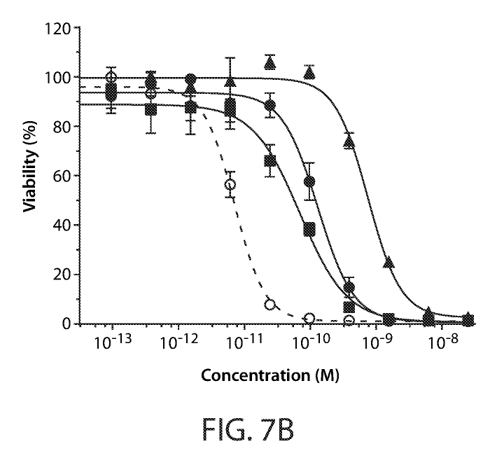

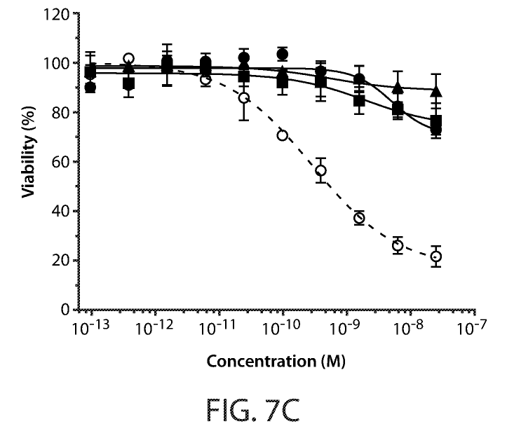

As shown in FIG. 6A, Jurkat cells will only undergo apoptosis in response to a cross-linked DR5 antibody. Cross-linked DR4 antibody, or DR4 and DR5 antibodies in the absence of cross-linking, have very little effect. However, Fc-scTRAIL is significantly more active than cross-linked anti-DR5 (FIG. 6B). The superiority of Fc-scTRAIL compared to cross-linked anti-DR4, anti-DR5 or the combination of anti-DR4 and 5 was seen across multiple cancer cell lines, such as DU1445, COLO205, and PANC1 cells (FIGS. 7A-C).

### Example 4: Apoptotic Activity of Fc-Sctrail is Dependent on Multivalency

### Methods

Variants of Fc-scTRAIL containing an inactivating Q205A substitution in 1, 2 or 3 of the TRAIL protomers were codon optimized for HEK293 expression, synthesized and cloned into the vector pCEP4 (Genscript, N.J.) using KpnI and NotI sites.

### Protein Expression

HEK-293F cells (FREESTYLE HEK-293 cells adapted for suspension culture, ThermoFisher (Cat.#R79007) stably expressing the anti-apoptotic protein Bcl-XL were grown in FREESTYLE F17 media (Gibco) containing 4 mM L-glutamine (Gibco) and 1% PLURONIC F-68 (Gibco) as suspension cultures in flasks with rotation (125 rpm). Cells were co-transfected with a 1 μg of plasmid DNA and 2.5 μg of linear 25 kDa polyethylenimine (Polysciences Inc.) per milliliter of cell culture. Density of cells at time of transfection was 1.5-2.0 e6 cells/ml. Cells were fed the following day with Tryptone Ni (Organotechnie) added to a final concentration of 5 mg/ml. Six days post transfection, cell cultures were centrifuged for 15 min at 5,000×g to pellet the cells. The supernatant media were decanted from the cells and filtered using 0.2 μm filter in preparation for purification.

### Cell Culture

H1993 cells were cultured in flasks with RPMI 1640 media (Gibco®) supplemented with 10% FBS, 100 units/ml penicillin, and 100 μg/ml streptomycin.

### Luminescent Cell Viability Assay

This assay was carried out as described in Example 1.

### Results

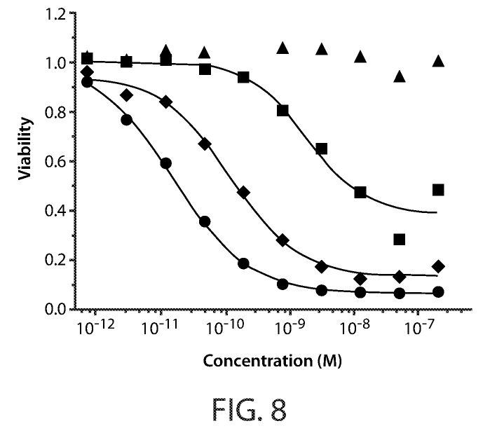

To confirm the correlation between apoptotic activity and the hexavalent nature of Fc-scTRAIL, knockout variants of Fc-scTRAIL were generated using Q205A mutations which is known to abrogate TRAIL binding to DR4 and 5 (Hymowitz et al. 2000, Biochemistry 39(4):633-40). The following variants were constructed: Fc-scTRAIL Q1 contains a single Q205A mutation in TRAIL protomer 1, Fc-scTRAIL Q2 contains two Q205A mutations in TRAIL protomers 1 and 2, and Fc-scTRAIL Q3 contains three Q205A mutations in all three TRAIL protomers. All 3 variants were compared against Fc-scTRAIL in a cell viability assay using H1993 cells. As shown in FIG. 8, with each reduction in valency, both the IC50 and maximum cell kill were reduced. Moreover, activity of Fc-scTRAIL Q2 with a valency of 2 is not unlike the activity seen for bivalent DR4 and 5 antibodies. This study highlights the advantage of the Fc-scTRAIL format compared to the agonist antibodies.

### Example 5: Stability of Fc-Sctrail in Thermal and Serum Stability Assays

### Methods

### Differential Scanning Fluorometry

Twenty-five micrograms of protein was analyzed using the Protein Thermal Shift Assay (Applied Biosystems) and fluorescence was detected using a VIIA 7 PCR system (Applied Biosystems) over a melt range of 25-99° C. The derivative-determined T_{M} was obtained using Protein Thermal Shift Software (Applied Biosystems).

### Mouse Serum Stability Assay

As an in vitro screen for serum stability, Fc-scTRAIL was incubated in 90% mouse serum (Sigma) at a final concentration of 1 μM for 0, 1, 3 and 7 days at 37° C. Samples are frozen at −80° C. and the end of incubation. The activity of Fc-scTRAIL was assessed in a cell viability assay using the colorectal carcinoma cell line, HCT116. Cells were seeded at 10,000 cells per well in 96 well tissue culture plate. Twenty-four hours later cells were incubated with a dilution series of the serum-incubated Fc-scTRAIL starting at 10 nM concentration. After a 24-hour treatment period, the amount of cellular ATP was detected using CELLTITER-GLO Assay (Promega) and measured on a SYNERGY H1 plate reader (BioTek). Luminescence was normalized to untreated controls and triplicates were averaged and plotted as a function of protein concentration. Non-linear regression was fitted using a 4 parameter least square fit using PRISM software (GraphPad).

### Results

### Fc-scTRAIL has Low Melting Temperature

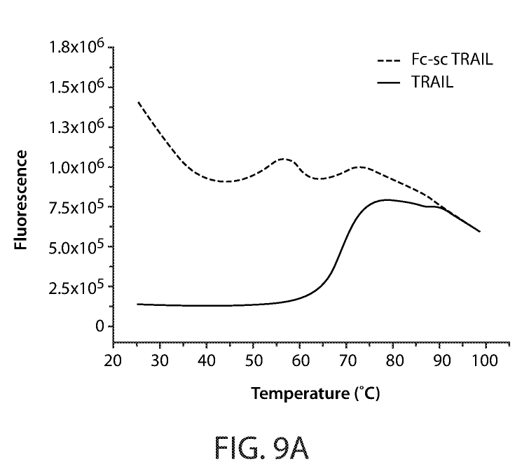

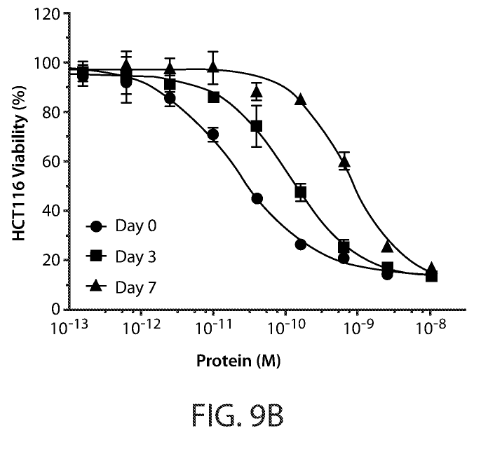

Despite significant improvements in pro-apoptotic activity compared to TRAIL or agonistic DR4 and DR5 antibodies, evidence of protein instability was observed for Fc-scTRAIL. As shown in FIG. 9A, the thermal stability of Fc-scTRAIL was determined by differential scanning fluorometry. Unexpectedly, the T_{M} of Fc-scTRAIL (53° C.) was observed to be significantly lower than TRAIL (71° C.). In addition, very high background interaction was observed between the Protein Thermal Shift Dye and Fc-scTRAIL but not TRAIL, an indication of the non-native structure of Fc-scTRAIL. Coordination of zinc is critical to the native folding of TRAIL (Hymowitz et al., Biochemistry, 2000 Feb. 1; 39(4):633-40). Therefore, the zinc content of purified Fc-scTRAIL was analyzed using inductive coupled plasma mass spectrometry (ICP-MS). The zinc content of a known concentration of Fc-scTRAIL was measured and based on the molar ratio, it was estimated that only 20% of Fc-scTRAIL contained zinc atom.

Fc-scTRAIL Loses Activity after Incubation in Mouse Serum

As an in vitro screen for serum stability, Fc-scTRAIL was incubated in 90% mouse serum (Sigma) at a final concentration of 1 μM for 0, 1, 3 and 7 days at 37° C. Subsequently, samples from each time-point were assessed in a cell viability assay using the colorectal carcinoma cell line, HCT116 and results are shown in FIG. 9B. Using the ratio of IC₅₀ for each time-point versus day 0, it was observed that there was negligible loss of activity after 24 hours (not shown), however, there was significant loss in activity following 3 days (5-fold) and 7 days (34-fold).

### Example 6: Identification of Mutations to Trail that Improve Stability

### Methods

### Yeast Library Construction

The nucleic acid sequence for TRAIL (114-281) was optimized for Saccharomyces cerevisiae using JCat codon adaptation tool (Grote et al, Nucl. Acids Res., 2005 v 33, Issue Suppl 2, pp W526-W531). The TRAIL nucleotide sequence is preceded by a V5 epitope tag and followed by the Tobacco Mosaic Virus (TMV) sequence and a FLAG epitope tag (SEQ ID NO:12). The TMV sequence refers to a 21 base pair sequence containing the stop codon found in the replicase gene of the TMV and was reported to have 30% read-through in Saccharomyces cerevisiae (Namy et al., EMBO Rep. 2001 September; 2(9):787-93). The TMV sequence was incorporated to allow the expression of both soluble TRAIL and the TRAIL/AGα fusion protein.

SEQ ID NO: 12
GAACGCGTGGAGGGGGTAAGCCTATACCTAACCCGCTGTTGGGGTTAGAC
AGCACGGGTGGATCCGTCAGAGAAAGAGGTCCACAAAGAGTCGCCGCCCA
CATAACAGGTACAAGAGGTAGAAGTAACACATTAAGTTCCCCAAATAGTA
AGAATGAAAAAGCTTTGGGTAGAAAGATTAACTCTTGGGAATCTTCAAGA
TCCGGTCATTCATTTTTGTCTAATTTGCACTTAAGAAACGGTGAATTAGT
CATTCATGAAAAGGGTTTCTACTACATCTATTCTCAAACATACTTCAGAT
TCCAAGAAGAAATTAAAGAAAACACCAAAAACGATAAGCAAATGGTACAA
TACATCTATAAGTACACAAGTTATCCAGACCCTATCTTGTTGATGAAGTC
TGCAAGAAACTCATGTTGGTCCAAGGATGCCGAATACGGTTTGTACTCTA
TCTATCAAGGTGGTATCTTCGAATTGAAGGAAAACGACAGAATCTTCGTT
TCAGTCACCAACGAACATTTGATTGATATGGACCACGAAGCATCCTTTTT
CGGTGCCTTTTTAGTAGGTGGAACACAATAGCAATTACAGGGCGCCTCAG
GATCTGGTGACTACAAGGACGACGATGACAAGGGTACCGGCGGGTCCGGA
GCTAGTGCCAAAAG
SEQ ID NO: 12 was amplified using forward
primers ET1
(GAACGCGTGGAGGGGGTAAGCCTATACCTA) (SEQ ID NO: 14)
and reverse primer ET2 (CTTTTGGCACTAGCTCCGGACCCGC)
(SEQ ID NO: 15) and cloned into pCR4 Blunt-TOPO
vector using ZERO BLUNT TOPO PCR
Cloning Kit to produce the plasmid V10.

Random mutagenesis was performed using the GENEMORPH II Random Mutagenesis Kit (Agilent Technologies). Twenty PCR reactions were set up, each containing 3 ng of V10 as template DNA and forward and reverse primers, ET31 (TACCTAACCCGCTGTTGGGGTTAGACAGCACGGGTGGATCCGTCAGAGAAAGAGGT CCACAAAGAGTCG) (SEQ ID NO:16) and ET32 (TTGTCATCGTCGTCCTTGTAGTCACCAGATCCTGAGGCGCCCTGTAATTGCTATTGT GTTCCACCTACTAAAAAGGCACCGAAAAAGGATG) (SEQ ID NO:17). Following 20 cycles of amplification, the PCR reactions were pooled and electrophoresed on 1% agarose gel. The PCR product was extracted and purified using WIZARD SV Gel and PCR Clean-Up kit (Promega). A secondary PCR amplification was then performed using the Q5 Hot Start High-Fidelity 2× Master Mix system (New England Biolabs). Purified primary PCR product was amplified for 8 cycles using forward and reverse primers, ET81 (TACCTAACCCGCTGTTGGGG) (SEQ ID NO:18) and ET82 (TTGTCATCGTCGTCCTTGTAGTC) (SEQ ID NO:19) and gel purified as before. The yeast display vector pMYD1000 (Xu et al. 2013) was digested with the restriction enzymes, BamHI and KasI, and gel purified. For electroporation, freshly prepared competent EBYZ cells (Xu et al. 2013) were incubated with the purified secondary PCR product and digested vector at a 3:1 ratio (w/w) and electroporated as described previously (Benatuil et al. 2010). The transformed library was grown overnight at 30° C. with shaking (225 rpm), aliquoted and stored at −80° C. Library size was estimated to be 1.1 e8 following serial dilution of cells on selective media.

### Yeast Library Panning

In preparation for library panning, the antigen, DR5-Fc (Abcam), was labeled with EZ-LINK Sulfo-NHS-biotin (ThermoFisher Scientific) according to manufacturer's instructions. We determined the ratio of ˜3 biotin molecules per protein. The library (1e10 cells) was grown in SDCAA media (dextrose-20 mg/ml, casamino acids-10 mg/ml, yeast nitrogen base—3.4 mg/ml, ammonium sulfate—10 mg/ml, Na₂HPO₄ —5.4 mg/ml and NaH₂PO₄ —7.4 mg/ml) for 24 hours at 30° C. with shaking (225 rpm). Cells were then pelleted and resuspended in SDGAA media (galactose—20 mg/ml, casamino acids—10 mg/ml, yeast nitrogen base—3.4 mg/ml, ammonium sulfate—10 mg/ml, Na₂HPO₄ —5.4 mg/ml and NaH₂PO₄ —7.4 mg/ml) and grown for an additional 48 hours at 20° C. with shaking to induce expression of the TRAIL on the yeast cell surface. First round of library panning was performed using magnetic cell sorting. Briefly, cells from the induced library (1e10) were incubated with biotin-labeled DR5-Fc (100 nM) for 1 hour at 25° C. and antigen binding cells were enriched using streptavidin beads and a magnetic column (Miltenyi Biotec). Cells were eluted from the magnetic column into SDCAA media and grown overnight followed by induction as before. Subsequent rounds of panning were performed using FACS. Induced cells from first round of panning were incubated with 100 nM biotin-labeled DR5-Fc and 1 μg/ml of anti-FLAG (Sigma) for 1 hour at 25° C. Cells were then washed with wash buffer (PBS, pH 7.4 containing 0.5% BSA) and incubated with 1 μg/ml of both goat anti-mouse Fc/Alexa488 (Invitrogen) and streptavidin/Alexa647 (Invitrogen) for 1 hour at 4° C. Labeled cells were sorted using FACSARIA III cell sorter (BD Biosciences). The top 5% of double positive cells were sorted into SDCAA media and expanded for the next round. In panning rounds 3 and 4, antigen was reduced to 20 and 5 nM, respectively. In both of the later panning rounds, the top 1.5% of double positive cells were taken forward to the next round.

Cells sorted from round 4 panning were plated on SDCAA media plates and grown for 72 hours at 30° C. Individual colonies were then used to inoculate 1 ml SDCAA cultures in 96-well plate. Cultures were grown and induced as before. Cells were then pelleted and incubated with either 10 nM of DR5-Fc or DR4-Fc (Abcam). Clones that displayed the highest level of binding to both receptors were sequenced.

Cloning of TRAIL Mutations into Fc-scTRAIL Format

Mutant TRAIL nucleotide sequences were first amplified using 3 pairs of forward and reverse primers for each of the 3 TRAIL monomer positions in the Fc-scTRAIL format (SEQ ID NO:11).

Position 1
ET62
(GGAGAGGGTCTCGAGGAGGCGGCAGTGGTGGAGGTGGATCTGGCGGAG
GAGGCTCTGTCAGAGAAAGAGGTCCACAAAGAGTCGC)
(SEQ ID NO: 29)
ET63
(TCTCTCGGTCTCCACTACCGCCACCTCCTGATCCTCCACCGCCACCTA
CTAAAAAGGCACCGAAAAAGGATGCT) (SEQ ID NO: 30)
Position 2
ET64
(GAGAGAGGTCTCGTAGTGGTGGCGGAGGTTCAGTCAGAGAAAGAGGT
CCACAAAGAGTCGC) (SEQ ID NO: 31)
ET65
(TCTCTCGGTCTCCTGAGCCTCCTCCGCCACTGCCACCGCCTCCACC
TACTAAAAAGGCACCGAAAAAGGATGCT) (SEQ ID NO: 32)
Position 3
ET66
(GAGAGAGGTCTCGCTCAGGCGGAGGTGGCAGTGTCAGAGAAAGAGG
TCCACAAAGAGTCGC) (SEQ ID NO: 33)
ET67
(TCTCTCGGTCTCCATTAACCTACTAAAAAGGCACCGAAAAAGGATGCT)
(SEQ ID NO: 34)
In addition, the human IgG1 Fc region was
synthesized (SEQ ID NO: 13) and amplified using
forward primer, ET160 (GTTCTAGGTCTCATGTGGGCTGA
TAAGACACATACATGCCCT) (SEQ ID NO: 20), and
reverse primer, ET161 (CACAATGGTCTCTTCCTCCACCC
GGCGACAAGCTTAGCGA) (SEQ ID NO: 21).
SEQ ID NO: 13
GTTCTAGGTCTCATGTGGGCTGATAAGACACATACATGCCCTCCATGTCC
CGCACCCGAGTTGCTTGGAGGACCTTCGGTGTTTCTTTTTCCCCCGAAGC
CAAAAGATACACTGATGATTTCACGGACGCCCGAGGTGACTTGTGTCGTC
GTGGACGTCAGCCACGAGGACCCAGAAGTCAAGTTTAACTGGTATGTAGA
TGGGGTGGAGGTACACAATGCGAAAACGAAACCGAGAGAGGAGCAGTACA
ATTCGACGTATAGGGTGGTCAGCGTGCTGACGGTGTTGCACCAGGACTGG
CTGAACGGGAAAGAGTATAAGTGCAAAGTGTCGAACAAGGCCCTCCCCGC
ACCCATCGAAAAGACGATATCCAAAGCCAAGGGCCAACCGCGCGAGCCGC
AAGTGTACACGCTGCCTCCCTCGCGAGAAGAGATGACCAAGAACCAGGTG
TCCCTTACGTGCTTGGTGAAAGGATTCTACCCTTCGGACATCGCCGTAGA
ATGGGAAAGCAATGGGCAGCCAGAGAACAATTACAAAACCACACCGCCTG
TGCTCGACTCGGACGGTTCCTTTTTCTTGTATTCCAAGTTGACAGTGGAC
AAGTCACGGTGGCAACAGGGGAACGTATTCTCGTGTTCCGTCATGCACGA
AGCGCTGCATAACCACTACACTCAGAAGTCGCTAAGCTTGTCGCCGGGTG
GAGGAAGAGACCATTGTG

Following 30 cycles of amplification, the three different TRAIL amplicons for each individual mutant were combined and gel purified as a pool. The human IgG1 Fc amplicon was gel purified separately. A combination restriction digest/ligation reaction was set up as follows: the TRAIL amplicons, Fc amplicon, and pSC4 vector were combined at a 3:1:1 molar ratio and incubated with 20 units of BsaI (New England Biolabs) and six units of T4 Ligase (Promega) in the presence of T4 Ligase buffer (Promega) and BSA (New England Biolabs). The reaction proceeded in a thermocycler with the following conditions:

Step 1—37° C. (2 min)
Step 2—16° C. (3 min)
Steps 1 and 2 were cycled 50 times followed by 50° C. (5 min) and 80° C. (5 min)

The reaction was transformed into competent 5-alpha E. coli cells (New England Biolabs) and plated on LB plates containing carbenicillin (Teknova). The next day colonies were selected and cultured for DNA sequencing and isolation.

### Protein Expression

Mutant Fc-scTRAIL proteins were expressed in HEK293 F cells stably expressing Bcl-XL and purified as described in Example 1.

### Differential Scanning Fluorometry

This assay was carried out as described in Example 5.

### Mouse Serum Stability Assay

This assay was carried out as described in Example 5.

### Results

Multiple Useful Mutations were Identified Via Yeast Display Selection

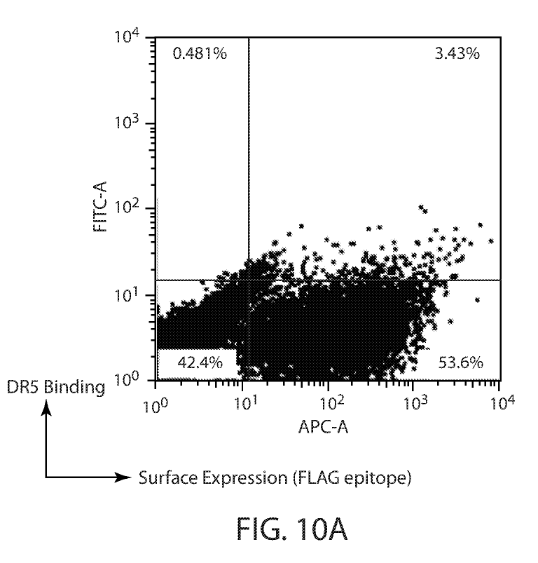

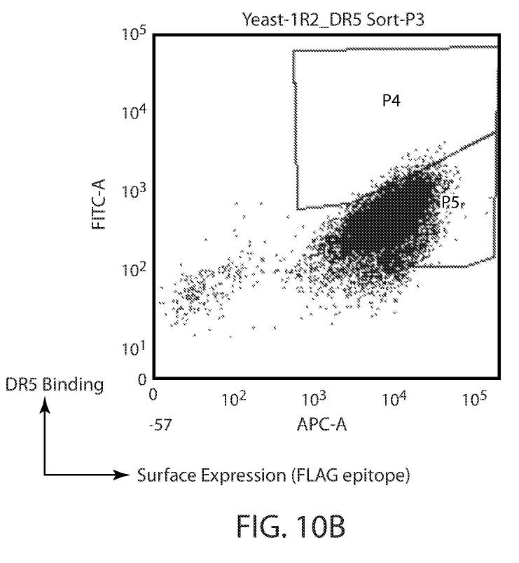

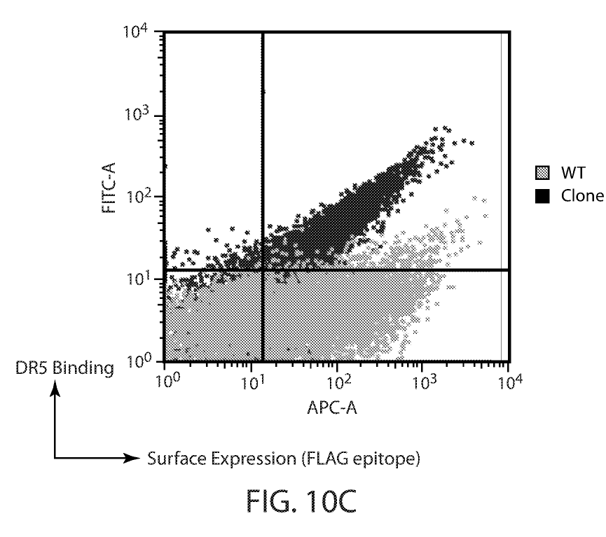

It was hypothesized that improving the stability of the TRAIL homotrimer would lead to an enhancement in the T_{M} of Fc-scTRAIL and improved serum stability. Therefore, identification of mutations within TRAIL were sought that would stabilize trimer formation and improve binding to DR5. A library of random mutations in TRAIL was generated using error prone PCR, and the library of TRAIL mutants was displayed on the surface of yeast. Sequencing of a small subset of the library revealed that 55% of the clones each contained 1-2 amino acid mutations. Flow cytometric analysis of the unselected library revealed a good expression of TRAIL on the surface as measured using anti-FLAG; however, there was little binding to the antigen, biotin-labeled DR5-Fc (FIG. 10A). After an initial round of panning using magnetic cell sorting followed by three subsequent rounds of panning using FACS and decreasing concentration of antigen, it was observed that the majority of clones were now positive for DR5 binding (FIG. 10B). The top 1% of the population sorted were grown and characterized individually. Shown in FIG. 10C is an exemplary clone that is significantly improved in DR5-Fc binding compared to the wild-type control.

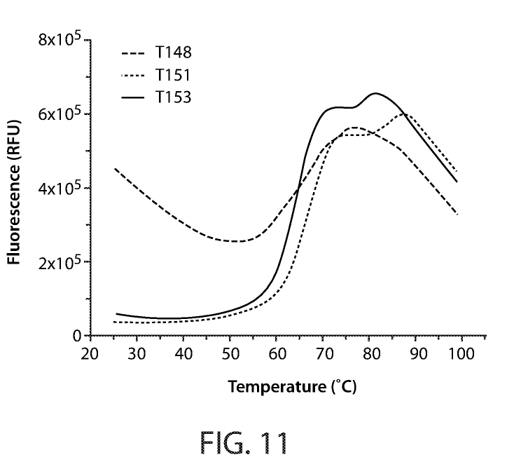

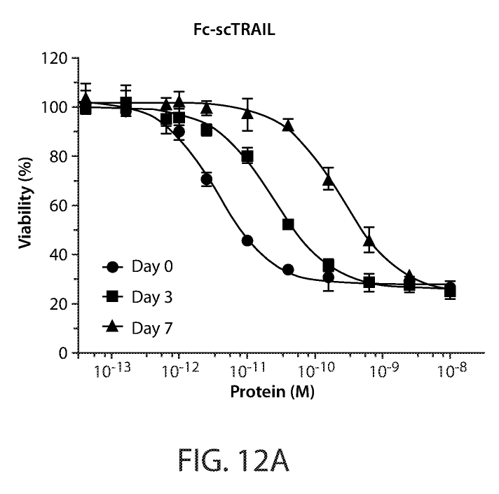

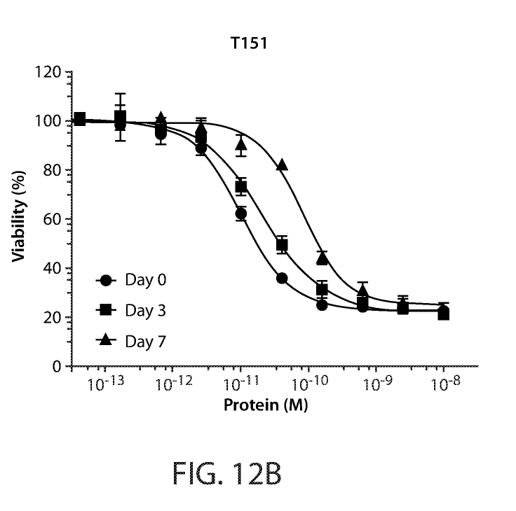

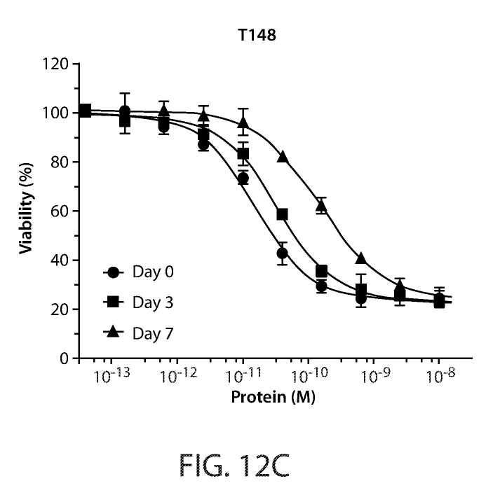

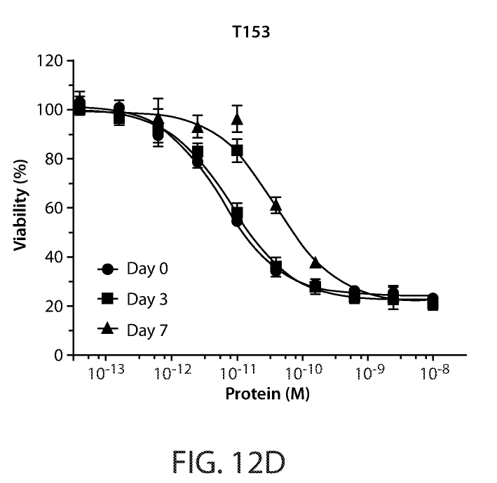

Individual clones that were confirmed to bind DR4-Fc, in addition to DR5-Fc, were then DNA sequenced. Mutant nucleotide sequences were then transferred into Fc-scTRAIL format for mammalian expression. Mutant Fc-scTRAIL proteins were expressed and purified as before and further characterized using the thermal shift assay. Shown in FIG. 11 are mutants T148, T151, and T153 that showed the most significant enhancement in T_{M}, (66-69° C.) compared to 48° C. for the wild-type Fc-scTRAIL. Interestingly, all three mutants contain the conservative amino acid substitution I247V. In the in vitro serum assay, all three mutants showed significant reduction in activity loss (6.5-10-fold) following a 7-day incubation in serum compared to the wild-type Fc-scTRAIL (FIGS. 12A-12D).

### Example 7: Mutations can Combine Additively or Synergistically for Increased Stability

### Methods

### Cloning of T183, T186 and T191

Mutant TRAIL nucleotide sequences were codon optimized for human expression using Jcat codon adaptation tool and synthesized (Genscript, N.J.). The synthesized DNA was then amplified using three pairs of forward and reverse primers for the three TRAIL monomer positions in Fc-scTRAIL (SEQ ID NO:11)

Position 1
ET154

(SEQ ID NO: 22)

(GTTCTAGGTCTCAAGGAGGCGGCAGTGGTGGAGGTG)
ET155

(SEQ ID NO: 23)

(CACAATGGTCTCTACCACCGCCCACCAGAAAGGCACCGA)
Position 2
ET156

(SEQ ID NO: 24)

(GTTCTAGGTCTCATGGTGGCGGCAGTGGTGGAGGTG)
ET157

(SEQ ID NO: 25)

(CACAATGGTCTCTCCCGCCGCCCACCAGAAAGGCACCGA)
Position 3
ET158

(SEQ ID NO: 26)

(GTTCTAGGTCTCACGGGGGCGGCAGTGGTGGAGGTG)
ET159

(SEQ ID NO: 27)

(CACAATGGTCTCTATTAGCCCACCAGAAAGGCACCGA)

Following 30 cycles of amplification, the three different TRAIL amplicons for each individual mutant were combined and gel purified as a pool. The TRAIL amplicons and the human IgG1 Fc amplicon were cloned into pSC4 vector as described above.

### Differential Scanning Fluorometry

This assay was carried out as described in Example 5.

### Mouse Serum Stability Assay

This assay was carried out as described in Example 5.

### Results

### Mutation Combinations Further Enhance Stability

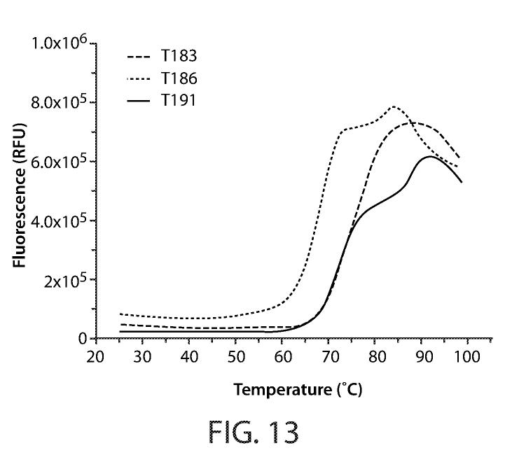

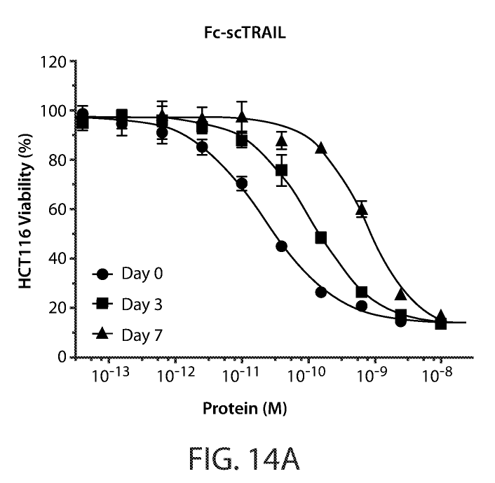

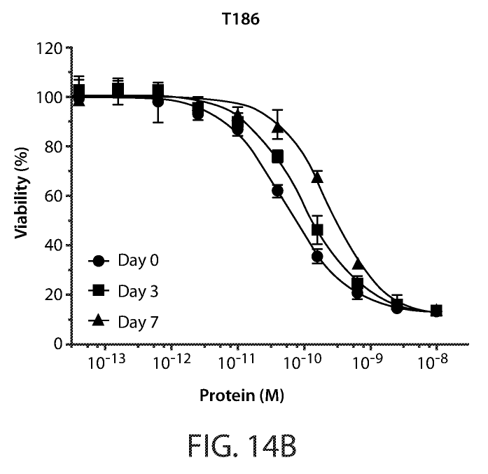

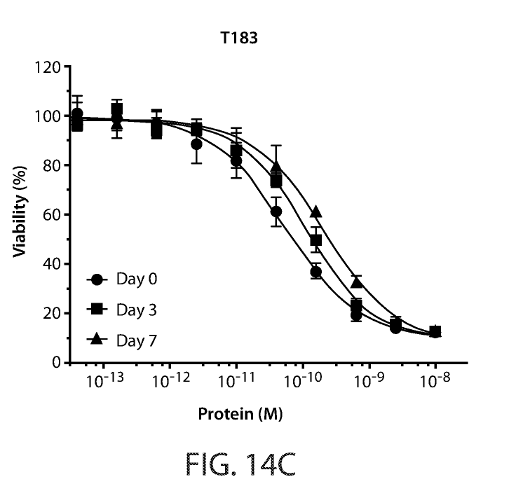

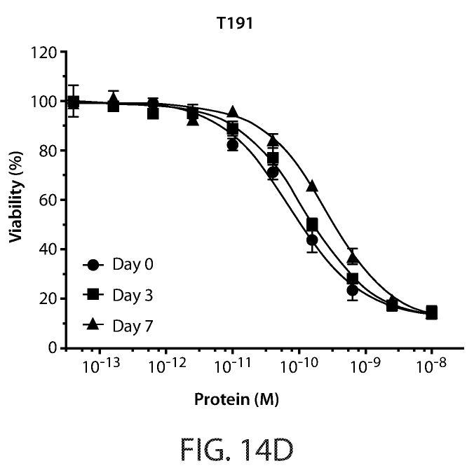

Based on the improvements in T_{M} and serum stability that were observed, the mutations from T148, T151, and T153 were combined to create 3 new combination mutants, T183, T186, and T191 (FIG. 13). Two additional mutations, Y213W and S215D, which have been shown to improve expression (Kelley et al. 2005) were also included. In the thermal shift assay, T183 and T191 displayed an even further enhanced T_{M} of 77 and 72° C., respectively, while the T_{M} of T186 was not significantly improved from the parental mutants. After a seven-day incubation in mouse serum, T183 and T186 showed 4-fold and 4.5-fold activity loss while T191 was the most improved showing <4-fold activity loss compared to wild-type (FIGS. 14A-14D).

### Example 8: In Vitro Activity of Exemplary Clone T191

### Methods

### Cell Culture

A549, DU145, and HOP62 cells were cultured in flasks with RPMI 1640 media (Gibco) supplemented with 10% FBS, 100 units/ml penicillin, and 100 μg/ml streptomycin. PANC-1 was cultured using DMEM media (Gibco) while SK-LU-1 was cultured using EMEM media (ATCC). Both media were also supplemented with 10% FBS, 100 units/ml penicillin, and 100 μg/mL streptomycin.

### Luminescent Cell Viability Assay

This assay was carried out as described in Example 7.

### Results

T191 Displays Enhanced Cell Killing in Comparison to rhTRAIL Soluble Ligand

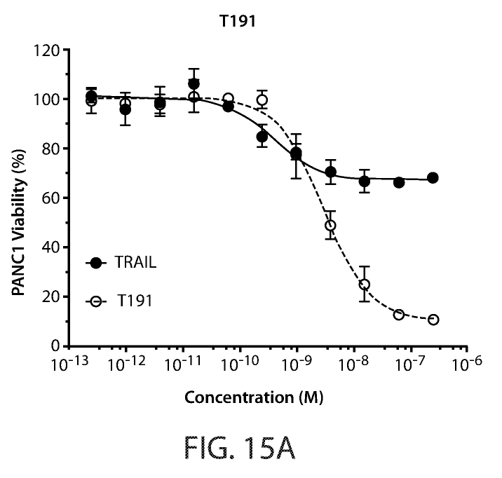

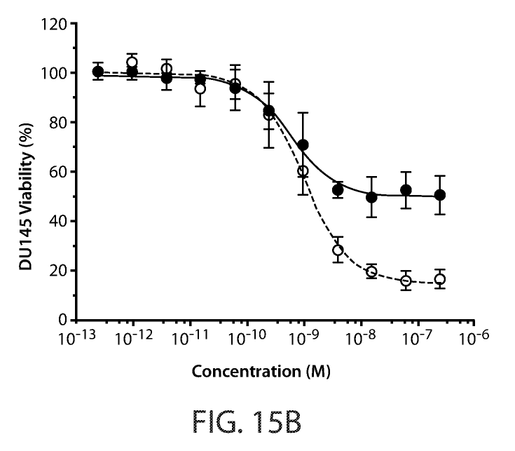

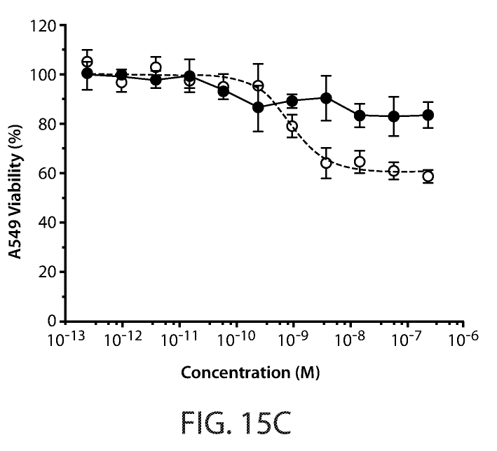

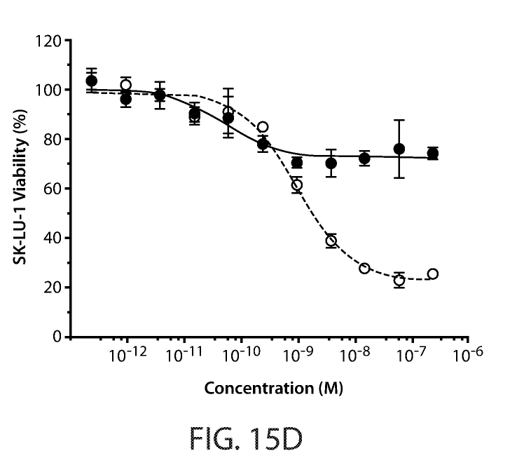

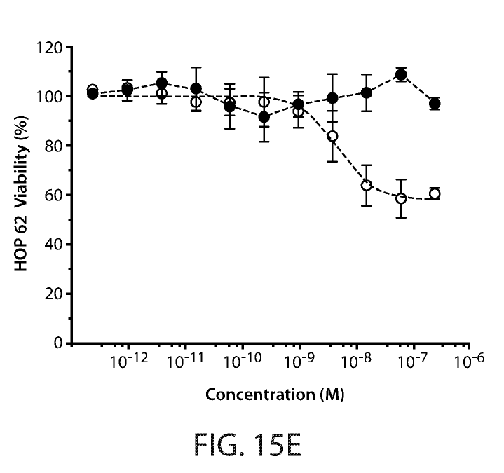

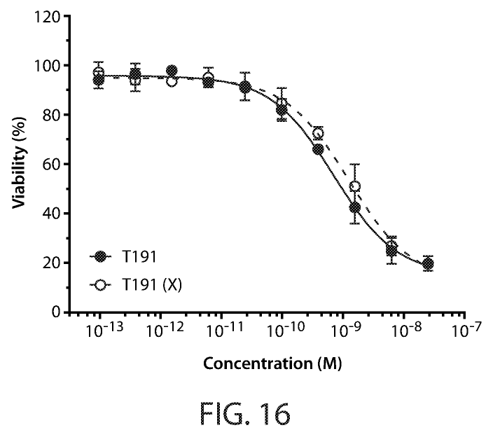

DU145, A549, PANC-1, HOP62, and SK-LU-1 cell lines are predominantly insensitive to native TRAIL. As shown in FIGS. 15A-15E, T191 shows not only improved IC₅₀s but more importantly enhanced maximum cell kill compared to TRAIL in all 5 cell lines. In DU145 cells, the addition of an equivalent molar concentration of anti-Fc antibody to provide Fc-mediated cross-linking had no effect on the activity of T191 in inducing cell death (FIG. 16).

### Example 9: Clone T191 Induces Apoptosis In Vitro

### Immunoblot Analysis of for Caspase-8, Bid, PARP and GAPDH

Cells were seeded in 6-well plates at 6.0×10⁵ cells/well in 2.7 mL media overnight. T191 (10 nM) with or without 10 nM of AFFINIPURE Goat Anti-Human IgG (Jackson ImmunoResearch Laboratories, Inc.) was added to each well and incubated for 2, 4, 8, or 24 hours at 37° C.

Untreated samples at 0 and 24 hours served as controls. At the end of incubation, medium from respective wells was collected and the cells were washed with ice cold Dulbecco's Phosphate Buffer Saline (PBS), pH7.4 (Gibco), trypsinized with 0.25% trypsin (Gibco), and collected into 15 ml tubes. Cells were pelleted and washed in ice cold PBS, and lysed in 250 μl of lysis buffer (RIPA Lysis and Extraction Buffer (Thermo Scientific)+Protease Inhibitor Cocktail (Sigma), Phosphatase Inhibitor Cocktail 2 (Sigma), 1 mM sodium orthovanadate, 10 mM sodium pyrophosphate, 50 μM phenylarsine, 10 μM bpV, 10 mM B-glycerophosphate, 1 M sodium fluoride). Cell lysates were incubated on ice for a minimum of 30 minutes; then transferred into 1.5 ml microcentrifuge tubes and stored at −80° C. Protein concentration was determined using the BCA Assay (Pierce), according to the manufacturer's protocol.

Protein samples (15 μg) were loaded onto a NUPAGE 4-12% Bis-Tris gel (Invitrogen) and separated by gel electrophoresis. Protein was transferred to nitrocellulose membrane using the IBLOT Dry Blotting System (Invitrogen). The membrane was blocked for 1 hour at room temperature in ODYSSEY Blocking Buffer (LI-COR), followed by an overnight incubation at 4° C. with primary antibodies diluted in 1:1 Odyssey blocking buffer/PBST (DPBS (Gibco) +0.1% TWEEN 20). Antibodies against the following proteins were used: Caspase-8 (Santa Cruz Biotechnology, sc-6136), BID (Cell Signaling Technology, #2002), PARP (Cell Signaling Technology, #9532), and GAPDH (Cell Signaling Technology, #2118). The next day, membranes were with PBST and incubated with secondary antibodies: IRDYE 800CW Goat anti-rabbit IgG (H+L) or IRDYE 800CW Donkey anti-goat IgG (H+L) (LI-COR) for 1 hour at room temperature. Membranes were washed once more in PBST and imaged using the ODYSSEY CLx Imaging system (LI-COR).

### T191 Rapidly Induces Apoptosis Through Caspase-8 Cleavage

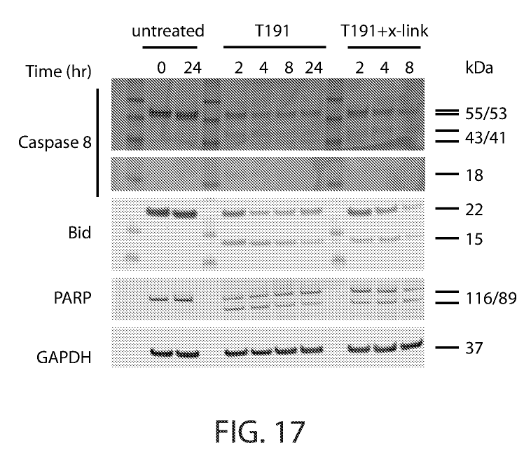

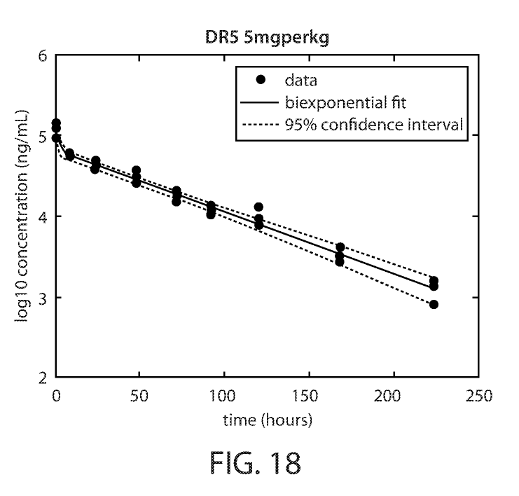

A time-course of T191 induced apoptosis in DU145 cells was investigated. Cells were treated with 10 nM of T191 for 2, 4, 8 and 24 hours, then lysed and analyzed by immunoblotting. We also investigated the effect of Fc crosslinking on T191 induced apoptosis by incubating T191 in the presence of anti-human Fc antibody. As shown in FIG. 17, induction of apoptosis was observed after only 2 hours of T191 treatment. Caspase 8 activation, as marked by detection of cleavage products at 43/41 kDa and 18 kDa, was observed after 2 hours of treatment, but not in untreated cells at either 0 or 24 hours. Total levels of caspase 8 decreased over the 24 hours as the pool is depleted after activation. Cleaved BID (15 kDa) supports the activity of caspase 8 as it is a substrate for the active Caspase. It also initiates the mitochondrial pathway for apoptosis. Cleaved PARP (89 kDa) is observed at all treatment time points and marks the execution of apoptosis in the cells. The kinetics of Caspase 8, BID, and PARP activation were not changed upon Fc-mediated cross-linking. These results demonstrate the rapid induction of apoptosis by T191 as the mechanism for changes in cell viability after treatment.

### Example 10: Half-Life Determination for Clone T191

### Methods

### Cloning of DR4 and DR5-his

The nucleotide sequence of DR4 (1-239) and DR5 (1-181) fused to a His6 tag (SEQ ID NO: 107) was synthesized and codon optimized for HEK293 expression (Genscript, N.J.). Both sequences were cloned into pCEP4 at the KpnI and XhoI restriction sites.

### Protein Expression

DR4-His and DR5-His proteins were expressed in HEK293F cells grown in FREESTYLE F17 media (Gibco) containing 4 mM L-glutamine (Gibco) and 1% PLURONIC F-68 (Gibco) as suspension cultures in flasks with rotation (125 rpm). Cells were transfected as described in Example 1.

### Protein Purification

PBS containing 800 mM imidazole, pH 7.0 was added to media containing DR4-His and DR5-His for a final concentration of ˜5 mM imidazole. The media was then loaded onto COMPLETE His-Tag Purification Resin (Roche) using ÄKTAEXPLORER (Amersham Biosciences) and was washed with PBS containing 0.5 M NaCl, pH 7.0. Both His-tagged proteins were then eluted using PBS containing 400 mM imidazole, pH 7.0, dialyzed overnight into PBS, pH 7.4 and stored at −80° C.

### Half-Life Determination in Mice

Five groups of four C57BL/6 mice (Charles River Laboratories) at 6-8 weeks old and 18-20 g body weight were each injected with either 5 mg/kg or 1 mg/kg of T191 in DPBS (Gibco) and bled at the specific time points: 0.5, 8.5, 24, 48, 72, 92, 120, 168, and 224 hours. Each mouse, with exception to the 0.5-hour group, was bled at two time-points, the earlier time-point being a tail vein bleed followed by a terminal cardiac bleed at the later time point. Mice in the 0.5-hour group received a single terminal bleed. Blood was collected in red-cap serum separator (Sarstedt Cat #16.441.100) and centrifuged at 12,500 rpm for 8 minutes at 4° C. in a microcentrifuge (Eppendorf). The serum was transferred to fresh 1.5 m microcentrifuge tubes and stored at −80° C.

T191 protein levels in the mouse serum were measured by ELISA. Plates (384-well) were coated overnight at room temperature with either 1 μg/ml DR4-His or DR5-His diluted in DPBS (Gibco). Plates are blocked with DPBS containing 2% bovine serum albumin (Sigma) for 1 hour at room temperature and then washed with PBST (DPBS+0.05% TWEEN-20). Serum samples serially diluted (10,000-500 fold) using dilution buffer (DPBS containing 2% BSA and 0.1% TWEEN 20/DPBS while freshly thawed T191 diluted in buffer (900-0.15 ng/ml) was used as standard. Samples were incubated with the coated receptors for 2 hours at room temperature. Plates are washed in PBST, then incubated with Peroxidase-conjugated AFFINIPURE Goat Anti-Human IgG (H+L) (Jackson ImmunoResearch Laboratories, Inc.) for 1 hour at room temperature. Plates are washed again with PBST and incubated with SUPERSIGNAL ELISA Pico Chemiluminescent Substrate (ThermoFisher Scientific). Luminescence was detected using the SYNERGY H1 Reader (BioTek). Raw luminescence was normalized to buffer only wells and then regressed to the standard curve using a 4-pt logistic curve. Regressed values are corrected by dilution factor, and then averaged to determine sample concentrations of T191.

Serum levels of T191 as a function of time (hours) were fit to a bi-exponential curve (y=Ae^{-αt}+Be^{-βt}), where y represents drug concentration, t represents time, and beta<alpha using MATLAB (Version 8.5.0.197613 (R2015a), License Number 518808) for each group of data (5 mg/kg and 1 mg/kg groups, DR5- or DR4-binding assay measurements). The fit was achieved using a non-linear least squares regression function (nlinfit.m in Matlab), and weights were applied to each serum drug concentration (biological replicate) to increase/decrease the influence of that value on the fitted model. The weight applied to each serum drug concentration at a given time was equal to the inverse of the standard deviation of all serum drug concentrations associated with that time point. The slope was used to calculate the terminal half-life (half-life=log(2)/beta).

### Results

### T191 has Extended Terminal Half-Life in Mice

To investigate whether T191 had improved pharmacokinetics in mice, C57BL/6 mice were injected at one of two doses, 1 and 5 mg/kg. Mice were bled at several time points (0.5, 8.5, 24, 48, 72, 92, 120, 168, and 224 hours) and the functional levels of T191 in the serum were determined by DR4 and DR5 binding ELISA. Drug concentration was then plotted as a function of time (FIGS. 15A-15E) and from the curve, the terminal half-lives for T191 were determined (Table 3).

TABLE 3
Terminal Half-life of T191 in C57BL/6 mice

| Half-life (hours) | DR5-based | DR4-based |
|---|---|---|
| (95% confidence interval) | ELISA | ELISA |
| 5 mg/kg bw dose | 39.4 | 43.5 |
| | (35.2-44.8) | (39.6-48.3) |
| 1 mg/kg bw dose | 33.7 | 41.1 |
| | (29.6-39.0) | (36.3-47.3) |

Values are consistent independent of dose and ELISA assay. The terminal half-life is greater than 30 hours, compared to the reported half-life of TRAIL of 3.6 minutes in mice (Kelley et al. 2001).

### Example 11: Efficacy of T191 in the COLO205 Xenograft Model

### Methods

### Proteins

Recombinant human TRAIL was purchased (Peprotech). The Fc-scTRAIL variant, T191, was expressed and purified as described above.

### COLO205 Xenograft Model

Nude mice (NU-Foxn1nu; Charles River Laboratories) at 6 weeks old and 18-20 g body weight were injected subcutaneously in the right flank with a suspension of COLO205 cells (3e6) in 50% MATRIGEL (Corning). Tumor measurements were made using a digital caliper and tumor volumes were calculated using the following equation: π/6(L×Ŵ2) with the “W” being the maximum width and the “L” being the maximum length. Once tumors were of sufficient size (250 mm³), mice were randomized into five groups (9 mice each) and injected two days later with either PBS pH 7.4, TRAIL, or T191 at the indicated doses and schedule (Table 4).

TABLE 4
Treatment groups for COLO205 xenograft study

| | | Day of |
|---|---|---|
| Group | Treatment and dose | treatment |
| A | PBS | 1, 3, 5 |
| B | TRAIL - 1 mg/kg | 1, 2, 3, 4, 5 |
| C | T191 - 1 mg/kg | 1, 2, 3, 4, 5 |
| D | T191 - 1 mg/kg | 1, 3, 5 |
| E | T191 - 5 mg/kg | 1 |

Tumor volumes and body weights were then monitored twice weekly for a total of 23 days. Following the last measurement, mice were bled and tumors were harvested for future histological evaluation. To determine the statistical differences between the treatment groups, one-way ANOVA analysis was performed using the fractional change in tumor volume for day 23 for each mouse.

### Results

### T191 Demonstrates Stronger Response in a COLO205 Xenograft Model at Equivalent Dosing

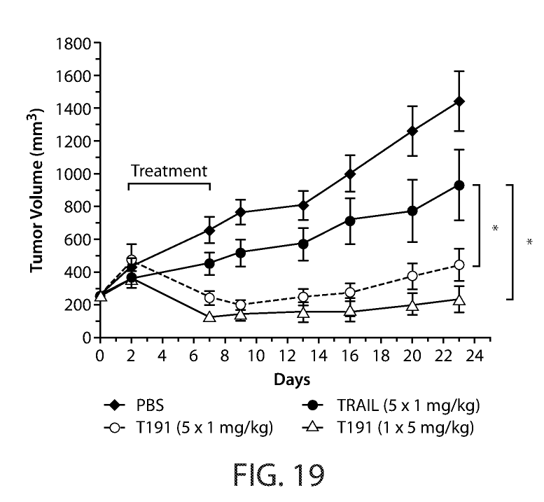

To investigate whether the increased in vitro activity and extended half-life of T191 translated into improved in vivo efficacy, T191 and TRAIL were compared in a COLO205 xenograft model. As shown in FIG. 19, tumors grew rapidly in mice treated with PBS only while five consecutive doses of TRAIL at 1 mg/kg delayed tumor growth modestly but was not determined to be statistically significant from the PBS control (Table 5). In contrast, five consecutive doses of T191 at 1 mg/kg resulted in initial regression and delayed outgrowth until day 16 of the study while a single dose of T191 at 5 mg/kg caused significant tumor regression and inhibited outgrowth for the duration of the 23-day study. Both T191 treatment groups were determined to be statistical different from PBS control and TRAIL treated mice (Table 5).

### Example 12: Efficacy of T191 in the HCC2998 and LS411N Xenograft Models

### Methods

### HCC2998 and LS411N Xenograft Models

Nude mice (NU-Foxn1nu; Charles River Laboratories) at 6 weeks old and 18-21 g body weight were injected subcutaneously in the right flank with a suspension of HCC2998 or LS411N cells (5e6) in 50% MATRIGEL (Corning). Tumor measurements were made using a digital caliper and tumor volumes were calculated using the following equation: n/6(L×Ŵ2) with the “W” being the maximum width and the “L” being the maximum length. Once tumors were of sufficient size (˜200 mm³), mice were randomized into two groups (5 mice each) and injected with either PBS pH 7.4, T191 at the indicated doses and schedule (Table 6).

TABLE 6
Treatment groups for HCC2998 or LS411N xenograft study

| Group | Treatment and dose | Day of treatment (post inoculation) |
|---|---|---|
| A | PBS-IP | 5, 12 |
| B | T191 - 5 mg/kg IP | 5, 12 |

Tumor volumes and body weights were monitored twice weekly for a total of 27 and 17 days post-treatment in the HCC2998 and LS411N models, respectively.

### Results

### T191 Demonstrates Tumor Regression in Both HCC2998 and LS411N Xenograft Models.

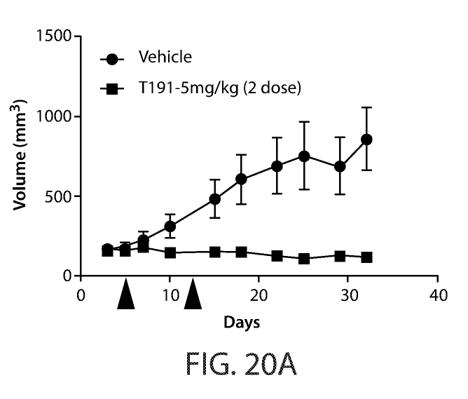

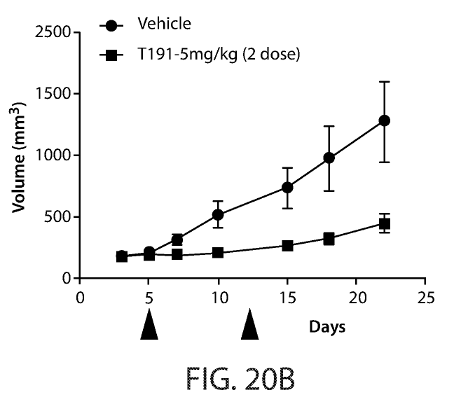

To further confirm the ability of T191 to suppress the tumor growth, the efficacy of this protein was tested and compared to PBS (control) in other colorectal xenograft models including HCC2998 and LS411N. As shown in FIGS. 20A-20B, tumors grew rapidly in control mice treated with PBS while 2 doses of T191 at 5 mg/kg inhibited the tumor growth in both models. T191 led to a stronger response in HCC2998 than LS411N which is consistent with its in vitro activity.

### Example 13: Back Mutation Analysis of T191 Variants

### Methods

### Protein Expression

Mutant Fc-scTRAIL proteins were cloned as described in Example 7 and expressed in HEK293 F cells stably expressing Bcl-XL and purified as described in Example 1.

### Differential Scanning Fluorometry

This assay was carried out as described in Example 5.

### Mouse Serum Stability Assay

This assay was carried out as described in Example 5.

### Results

Mutations in T191 were individually back-mutated to the wild-type sequence. Separate Fc-scTRAIL variants containing all combinations of substitutions found in T191 were generated. The full amino acid sequences of variants T202, T203, T207, T208, T209, T210, and T211 are shown in Table 9 below.

The thermal melts of Fc-scTRAIL variants were determined by differential scanning fluorimetry (Table 7). The majority of the variants (T202, T203, T207, T208, T210, and T211) showed comparable thermal melting temperature to T191, with the exception of the variant T209 which displayed T_{M} of 64.3° C.

TABLE 7

| Protein | Substitutions in TRAIL | | | | | | Thermal Melt (° C.) |
|---|---|---|---|---|---|---|---|
| Fc-scTRAIL | | | | | | | 53.3 |
| T148 | R121I | | | I247V | | | 66.3 |
| T151 | | | N228S | I247V | | | 69.3 |
| T153 | | R130G | | I247V | | | 67,1 |
| T182 | | | | | Y213W | S215D | 56.5 |
| T183 | R121I | R130G | | I247V | Y213W | S215D | 76.5 |
| T186 | | R130G | | I247V | Y213W | S215D | 68.5 |
| T191 | | R130G | N228S | I247V | Y213W | S215D | 71.8 |
| T196 | R121I | R130G | | I247V | | | 73.2 |
| T202 | | R130G | N228S | I247V | | | 68.7 |
| T203 | R121I | R130G | N228S | I247V | | | 70.4 |
| T207 | R121I | | N228S | I247V | | | 71.5 |
| T208 | R121I | R130G | | | | | 70.3 |
| T209 | R121I | R130G | N228S | | | | 64.3 |
| T210 | R121I | | N228S | | | | 70.6 |
| T211 | | R130G | N228S | | | | 71.3 |

Serum stability was measured by incubation of the variants in mouse serum for 0 and 7 days and the activity was subsequently measured in a HCT116 cell viability assay and described using IC50 (Table 8, columns 2 and 4). The activity of each variant compared to wild-type is represented by a ratio of variant IC₅₀/Fc-scTRAIL IC₅₀ at day 0 (Table 9, column 3). The majority of the variants showed improved activity as observed by their IC50 at day 0. The loss in activity after 7 days in mouse serum is represented by the ratio of IC50 day 7/IC50 day 0 for each protein (Table 9, column 5).

TABLE 8

| | | Fold-change | | Fold |
|---|---|---|---|---|
| | IC₅₀ (M) | compared to Fc- | IC₅₀ (M) | change Day 7 |
| Protein | at Day 0 | scTRAIL | at Day 7 | Day 0 |
| Fc-scTRAIL | 2.69E−11 | — | 9.46E−10 | 35.2 |
| T151 | 1.48E−11 | 0.5 | 1.00E−10 | 6.8 |
| T153 | 7.76E−12 | 0.3 | 6.17E−11 | 7.9 |
| T191 | 7.24E−11 | 2.7 | 3.41E−10 | 4.7 |
| T202 | 1.15E−11 | 0.4 | 8.71E−11 | 7.6 |
| T203 | 1.02E−11 | 0.4 | 8.13E−11 | 7.9 |
| T207 | 1.62E−11 | 0.6 | 3.40E−10 | 21.0 |
| T208 | 1.17E−11 | 0.4 | 1.30E−10 | 11.1 |
| T209 | 1.48E−11 | 0.5 | 1.08E−10 | 7.3 |
| T210 | 2.00E−11 | 0.7 | 1.62E−10 | 8.1 |
| T211 | 1.23E−10 | 4.6 | 8.59E−10 | 7.0 |

TABLE 9

| Variant | Amino Acid Sequence |
|---|---|
| T148 - Fc-TRAIL | DKTHTCPPCPAPELLGGPSVFLFPPKPKDTLMISRTPEVTCVVVDVS |
| (SEQ ID NO: 35) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARNSCWSKDAEYG |
| | LYSIYQGGVFELKENDRIFVSVTNEHLIDMDHEASFFGAFLVGGGG |
| | GSGGGGSGGGGSVRERGPQIVAAHITGTRGRSNTLSSPNSKNEKAL |
| | GRKINSWESSRSGHSFLSNLHLRNGELVIHEKGFYYIYSQTYFRFQE |
| | EIKENTKNDKQMVQYIYKYTSYPDPILLMKSARNSCWSKDAEYGL |
| | YSIYQGGVFELKENDRIFVSVTNEHLIDMDHEASFFGAFLVGGGGG |
| | SGGGGSGGGGSVRERGPQIVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARNSCWSKDAEYGLYS |
| | IYQGGVFELKENDRIFVSVTNEHLIDMDHEASFFGAFLVG |
| T151 - Fc-TRAIL | DKTHTCPPCPAPELLGGPSVFLFPPKPKDTLMISRTPEVTCVVVDVS |
| (SEQ ID NO: 36) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGVFELKENDRIFVSVTNEHLIDMDHEASFFGAFLVGGGGG |
| | SGGGGSGGGGSVRERGPQRVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARSSCWSKDAEYGLYSI |
| | YQGGVFELKENDRIFVSVTNEHLIDMDHEASFFGAFLVGGGGGSGG |
| | GGSGGGGSVRERGPQRVAAHITGTRGRSNTLSSPNSKNEKALGRKI |
| | NSWESSRSGHSFLSNLHLRNGELVIHEKGFYYIYSQTYFRFQEEIKE |
| | NTKNDKQMVQYIYKYTSYPDPILLMKSARSSCWSKDAEYGLYSIY |
| | QGGVFELKENDRIFVSVTNEHLIDMDHEASFFGAFLVG |
| T153 - Fc-TRAIL | DKTHTCPPCPAPELLGGPSVFLFPPKPKDTLMISRTPEVTCVVVDVS |
| (SEQ ID NO: 37) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVG |
| T183 - Fc-TRAIL | DKTHTCPPCPAPELLGGPSVFLFPPKPKDTLMISRTPEVTCVVVDVS |
| (SEQ ID NO: 38) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTGGRSNTLSSPNSKNEKA |
| | LGRKINSWESSRSGHSFLSNLHLRNGELVIHEKGFYYIYSQTYFRFQ |
| | EEIKENTKNDKQMVQYIYKWTDYPDPILLMKSARNSCWSKDAEYG |
| | LYSIYQGGVFELKENDRIFVSVTNEHLIDMDHEASFFGAFLVGGGG |
| | GSGGGGSGGGGSVRERGPQIVAAHITGTGGRSNTLSSPNSKNEKAL |
| | GRKINSWESSRSGHSFLSNLHLRNGELVIHEKGFYYIYSQTYFRFQE |
| | EIKENTKNDKQMVQYIYKWTDYPDPILLMKSARNSCWSKDAEYGL |
| | YSIYQGGVFELKENDRIFVSVTNEHLIDMDHEASFFGAFLVGGGGG |
| | SGGGGSGGGGSVRERGPQIVAAHITGTGGRSNTLSSPNSKNEKALG |
| | RKINSWESSRSGHSFLSNLHLRNGELVIHEKGFYYIYSQTYFRFQEEI |
| | KENTKNDKQMVQYIYKWTDYPDPILLMKSARNSCWSKDAEYGLY |
| | SIYQGGVFELKENDRIFVSVTNEHLIDMDHEASFFGAFLVG |
| T186 - Fc-TRAIL | DKTHTCPPCPAPELLGGPSVFLFPPKPKDTLMISRTPEVTCVVVDVS |
| (SEQ ID NO: 39) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARNSCWSKDAEY |
| | GLYSIYQGGVFELKENDRIFVSVTNEHLIDMDHEASFFGAFLVG |
| T191 - Fc-TRAIL | DKTHTCPPCPAPELLGGPSVFLFPPKPKDTLMISRTPEVTCVVVDVS |
| (SEQ ID NO: 40) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARSSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARSSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKWTDYPDPILLMKSARSSCWSKDAEY |
| | GLYSIYQGGVFELKENDRIFVSVTNEHLIDMDHEASFFGAFLVG |
| T202 - Fc-TRAIL | DKTHTCPPCPAPELLGGPSVFLFPPKPKDTLMISRTPEVTCVVVDVS |
| (SEQ ID NO: 41) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARSSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARSSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARSSCWSKDAEY |
| | GLYSIYQGGVFELKENDRIFVSVTNEHLIDMDHEASFFGAFLVG |
| T203 - Fc-TRAIL | DKTHTCPPCPAPELLGGPSVFLFPPKPKDTLMISRTPEVTCVVVDVS |
| (SEQ ID NO: 42) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTGGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGVFELKENDRIFVSVTNEHLIDMDHEASFFGAFLVGGGGG |
| | SGGGGSGGGGSVRERGPQIVAAHITGTGGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARSSCWSKDAEYGLYSI |
| | YQGGVFELKENDRIFVSVTNEHLIDMDHEASFFGAFLVGGGGGSGG |
| | GGSGGGGSVRERGPQIVAAHITGTGGRSNTLSSPNSKNEKALGRKI |
| | NSWESSRSGHSFLSNLHLRNGELVIHEKGFYYIYSQTYFRFQEEIKE |
| | NTKNDKQMVQYIYKYTSYPDPILLMKSARSSCWSKDAEYGLYSIY |
| | QGGVFELKENDRIFVSVTNEHLIDMDHEASFFGAFLVG |
| T204 - Fc-TRAIL | DKTHTCPPCPAPELLGGPSVFLFPPKPKDTLMISRTPEVTCVVVDVS |
| (SEQ ID NO: 43) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARNSCWSKDAEYG |
| | LYSIYQGGVFELKENDRIFVSVTNEHLIDMDHEASFFGAFLVGGGG |
| | GSGGGGSGGGGSVRERGPQIVAAHITGTRGRSNTLSSPNSKNEKAL |
| | GRKINSWESSRSGHSFLSNLHLRNGELVIHEKGFYYIYSQTYFRFQE |
| | EIKENTKNDKQMVQYIYKYTSYPDPILLMKSARNSCWSKDAEYGL |
| | YSIYQGGVFELKENDRIFVSVTNEHLIDMDHEASFFGAFLVGGGGG |
| | SGGGGSGGGGSVRERGPQIVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARNSCWSKDAEYGLYS |
| | IYQGGVFELKENDRIFVSVTNEHLIDMDHEASFFGAFLVG |
| T205 - Fc-TRAIL | DKTHTCPPCPAPELLGGPSVFLFPPKPKDTLMISRTPEVTCVVVDVS |
| (SEQ ID NO: 44) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARNSCWSKDAEY |
| | GLYSIYQGGVFELKENDRIFVSVTNEHLIDMDHEASFFGAFLVG |
| T206 - Fc-TRAIL | DKTHTCPPCPAPELLGGPSVFLFPPKPKDTLMISRTPEVTCVVVDVS |
| (SEQ ID NO: 45) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGVFELKENDRIFVSVTNEHLIDMDHEASFFGAFLVGGGGG |
| | SGGGGSGGGGSVRERGPQRVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARSSCWSKDAEYGLYSI |
| | YQGGVFELKENDRIFVSVTNEHLIDMDHEASFFGAFLVGGGGGSGG |
| | GGSGGGGSVRERGPQRVAAHITGTRGRSNTLSSPNSKNEKALGRKI |
| | NSWESSRSGHSFLSNLHLRNGELVIHEKGFYYIYSQTYFRFQEEIKE |
| | NTKNDKQMVQYIYKYTSYPDPILLMKSARSSCWSKDAEYGLYSIY |
| | QGGVFELKENDRIFVSVTNEHLIDMDHEASFFGAFLVG |
| T207 - Fc-TRAIL | DKTHTCPPCPAPELLGGPSVFLFPPKPKDTLMISRTPEVTCVVVDVS |
| (SEQ ID NO: 46) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGVFELKENDRIFVSVTNEHLIDMDHEASFFGAFLVGGGGG |
| | SGGGGSGGGGSVRERGPQIVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARSSCWSKDAEYGLYSI |
| | YQGGVFELKENDRIFVSVTNEHLIDMDHEASFFGAFLVGGGGGSGG |
| | GGSGGGGSVRERGPQIVAAHITGTRGRSNTLSSPNSKNEKALGRKIN |
| | SWESSRSGHSFLSNLHLRNGELVIHEKGFYYIYSQTYFRFQEEIKENT |
| | KNDKQMVQYIYKYTSYPDPILLMKSARSSCWSKDAEYGLYSIYQG |
| | GVFELKENDRIFVSVTNEHLIDMDHEASFFGAFLVG |
| T208 - Fc-TRAIL | DKTHTCPPCPAPELLGGPSVFLFPPKPKDTLMISRTPEVTCVVVDVS |
| (SEQ ID NO: 47) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTGGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARNSCWSKDAEYG |
| | LYSIYQGGIFELKENDRIFVSVTNEHLIDMDHEASFFGAFLVGGGGG |
| | SGGGGSGGGGSVRERGPQIVAAHITGTGGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARNSCWSKDAEYGLYS |
| | IYQGGIFELKENDRIFVSVTNEHLIDMDHEASFFGAFLVGGGGGSGG |
| | GGSGGGGSVRERGPQIVAAHITGTGGRSNTLSSPNSKNEKALGRKI |
| | NSWESSRSGHSFLSNLHLRNGELVIHEKGFYYIYSQTYFRFQEEIKE |
| | NTKNDKQMVQYIYKYTSYPDPILLMKSARNSCWSKDAEYGLYSIY |
| | QGGIFELKENDRIFVSVTNEHLIDMDHEASFFGAFLVG |
| T209 - Fc-TRAIL | DKTHTCPPCPAPELLGGPSVFLFPPKPKDTLMISRTPEVTCVVVDVS |
| (SEQ ID NO: 48) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTGGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGIFELKENDRIFVSVTNEHLIDMDHEASFFGAFLVGGGGGS |
| | GGGGSGGGGSVRERGPQIVAAHITGTGGRSNTLSSPNSKNEKALGR |
| | KINSWESSRSGHSFLSNLHLRNGELVIHEKGFYYIYSQTYFRFQEEIK |
| | ENTKNDKQMVQYIYKYTSYPDPILLMKSARSSCWSKDAEYGLYSIY |
| | QGGIFELKENDRIFVSVTNEHLIDMDHEASFFGAFLVGGGGGSGGG |
| | GSGGGGSVRERGPQIVAAHITGTGGRSNTLSSPNSKNEKALGRKINS |
| | WESSRSGHSFLSNLHLRNGELVIHEKGFYYIYSQTYFRFQEEIKENT |
| | KNDKQMVQYIYKYTSYPDPILLMKSARSSCWSKDAEYGLYSIYQG |
| | GIFELKENDRIFVSVTNEHLIDMDHEASFFGAFLVG |
| T210 - Fc-TRAIL | DKTHTCPPCPAPELLGGPSVFLFPPKPKDTLMISRTPEVTCVVVDVS |
| (SEQ ID NO: 49) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARSSCWSKDAEYGL |
| | YSIYQGGIFELKENDRIFVSVTNEHLIDMDHEASFFGAFLVGGGGGS |
| | GGGGSGGGGSVRERGPQIVAAHITGTRGRSNTLSSPNSKNEKALGR |
| | KINSWESSRSGHSFLSNLHLRNGELVIHEKGFYYIYSQTYFRFQEEIK |
| | ENTKNDKQMVQYIYKYTSYPDPILLMKSARSSCWSKDAEYGLYSIY |
| | QGGIFELKENDRIFVSVTNEHLIDMDHEASFFGAFLVGGGGGSGGG |
| | GSGGGGSVRERGPQIVAAHITGTRGRSNTLSSPNSKNEKALGRKINS |
| | WESSRSGHSFLSNLHLRNGELVIHEKGFYYIYSQTYFRFQEEIKENT |
| | KNDKQMVQYIYKYTSYPDPILLMKSARSSCWSKDAEYGLYSIYQG |
| | GIFELKENDRIFVSVTNEHLIDMDHEASFFGAFLVG |
| T211 - Fc-TRAIL | DKTHTCPPCPAPELLGGPSVFLFPPKPKDTLMISRTPEVTCVVVDVS |
| (SEQ ID NO: 50) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTGGRSNTLSSPNSKNEK |
| | ALGRKINSWESSRSGHSFLSNLHLRNGELVIHEKGFYYIYSQTYFRF |
| | QEEIKENTKNDKQMVQYIYKYTSYPDPILLMKSARSSCWSKDAEY |
| | GLYSIYQGGIFELKENDRIFVSVTNEHLIDMDHEASFFGAFLVGGGG |
| | GSGGGGSGGGGSVRERGPQRVAAHITGTGGRSNTLSSPNSKNEKAL |
| | GRKINSWESSRSGHSFLSNLHLRNGELVIHEKGFYYIYSQTYFRFQE |
| | EIKENTKNDKQMVQYIYKYTSYPDPILLMKSARSSCWSKDAEYGL |
| | YSIYQGGIFELKENDRIFVSVTNEHLIDMDHEASFFGAFLVGGGGGS |
| | GGGGSGGGGSVRERGPQRVAAHITGTGGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARSSCWSKDAEYGLYSI |
| | YQGGIFELKENDRIFVSVTNEHLIDMDHEASFFGAFLVG |
| T182 - Fc-TRAIL | DKTHTCPPCPAPELLGGPSVFLFPPKPKDTLMISRTPEVTCVVVDVS |
| (SEQ ID NO: 100) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQRVAAHITGTRGRSNTLSSPNSKNEKA |
| | LGRKINSWESSRSGHSFLSNLHLRNGELVIHEKGFYYIYSQTYFRFQ |
| | EEIKENTKNDKQMVQYIYKWTDYPDPILLMKSARNSCWSKDAEYG |
| | LYSIYQGGIFELKENDRIFVSVTNEHLIDMDHEASFFGAFLVGGGGG |
| | SGGGGSGGGGSVRERGPQRVAAHITGTRGRSNTLSSPNSKNEKALG |
| | RKINSWESSRSGHSFLSNLHLRNGELVIHEKGFYYIYSQTYFRFQEEI |
| | KENTKNDKQMVQYIYKWTDYPDPILLMKSARNSCWSKDAEYGLY |
| | SIYQGGIFELKENDRIFVSVTNEHLIDMDHEASFFGAFLVGGGGGSG |
| | GGGSGGGGSVRERGPQRVAAHITGTRGRSNTLSSPNSKNEKALGRK |
| | INSWESSRSGHSFLSNLHLRNGELVIHEKGFYYIYSQTYFRFQEEIKE |
| | NTKNDKQMVQYIYKWTDYPDPILLMKSARNSCWSKDAEYGLYSIY |
| | QGGIFELKENDRIFVSVTNEHLIDMDHEASFFGAFLVG |
| T196 - Fc-TRAIL | DKTHTCPPCPAPELLGGPSVFLFPPKPKDTLMISRTPEVTCVVVDVS |
| (SEQ ID NO: 101) | HEDPEVKFNWYVDGVEVHNAKTKPREEQYNSTYRVVSVLTVLHQ |
| | DWLNGKEYKCKVSNKALPAPIEKTISKAKGQPREPQVYTLPPSREE |
| | MTKNQVSLTCLVKGFYPSDIAVEWESNGQPENNYKTTPPVLDSDGS |
| | FFLYSKLTVDKSRWQQGNVFSCSVMHEALHNHYTQKSLSLSPGGG |
| | GGSGGGGSGGGGSVRERGPQIVAAHITGTGGRSNTLSSPNSKNEKA |
| | LGRKINSWESSRSGHSFLSNLHLRNGELVIHEKGFYYIYSQTYFRFQ |
| | EEIKENTKNDKQMVQYIYKYTSYPDPILLMKSARNSCWSKDAEYG |
| | LYSIYQGGVFELKENDRIFVSVTNEHLIDMDHEASFFGAFLVGGGG |
| | GSGGGGSGGGGSVRERGPQIVAAHITGTGGRSNTLSSPNSKNEKAL |
| | GRKINSWESSRSGHSFLSNLHLRNGELVIHEKGFYYIYSQTYFRFQE |
| | EIKENTKNDKQMVQYIYKYTSYPDPILLMKSARNSCWSKDAEYGL |
| | YSIYQGGVFELKENDRIFVSVTNEHLIDMDHEASFFGAFLVGGGGG |
| | SGGGGSGGGGSVRERGPQIVAAHITGTGGRSNTLSSPNSKNEKALG |
| | RKINSWESSRSGHSFLSNLHLRNGELVIHEKGFYYIYSQTYFRFQEEI |
| | KENTKNDKQMVQYIYKYTSYPDPILLMKSARNSCWSKDAEYGLYS |
| | IYQGGVFELKENDRIFVSVTNEHLIDMDHEASFFGAFLVG |

TABLE 10

| Variant | Nucleic Acid Sequence |
|---|---|
| T148 | ATGGCCTGGCGGCTGTGGTGGCTGCTGCTCCTGCTCCTGTTGCTTTGGCCTATGGTGTGG |
| (SEQ ID NO: 51) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTCAGAGAAAGAGGTCCACAAATAGTCGCCGCCCACA |
| | TAACAGGTACAAGAGGTAGAAGTAACACATTAAGTTCCCCAAATAGTAAGAATGAAAAA |
| | GCTTTGGGTAGAAAGATTAACTCTTGGGAATCTTCAAGATCCGGTCATTCATTTTTGTCTA |
| | ATTTGCACTTAAGAAACGGTGAATTAGTCATTCATGAAAAGGGTTTCTACTACATCTATTC |
| | TCAAACATACTTCAGATTCCAAGAAGAAATTAAAGAAAACACCAAAAACGATAAGCAAA |
| | TGGTACAATACATCTATAAGTACACAAGTTATCCAGACCCTATCTTGTTGATGAAGTCTG |
| | CAAGAAACTCATGTTGGTCCAAGGATGCCGAATACGGTTTGTACTCTATCTATCAAGGTG |
| | GTGTCTTCGAATTGAAGGAAAACGACAGAATCTTCGTTTCAGTCACCAACGAACATTTGA |
| | TTGATATGGACCACGAAGCATCCTTTTTCGGTGCCTTTTTAGTAGGTGGCGGTGGAGGAT |
| | CAGGAGGTGGCGGTAGTGGTGGCGGAGGTTCAGTCAGAGAAAGAGGTCCACAAATAG |
| | TCGCCGCCCACATAACAGGTACAAGAGGTAGAAGTAACACATTAAGTTCCCCAAATAGT |
| | AAGAATGAAAAAGCTTTGGGTAGAAAGATTAACTCTTGGGAATCTTCAAGATCCGGTCA |
| | TTCATTTTTGTCTAATTTGCACTTAAGAAACGGTGAATTAGTCATTCATGAAAAGGGTTTC |
| | TACTACATCTATTCTCAAACATACTTCAGATTCCAAGAAGAAATTAAAGAAAACACCAAA |
| | AACGATAAGCAAATGGTACAATACATCTATAAGTACACAAGTTATCCAGACCCTATCTTG |
| | TTGATGAAGTCTGCAAGAAACTCATGTTGGTCCAAGGATGCCGAATACGGTTTGTACTCT |
| | ATCTATCAAGGTGGTGTCTTCGAATTGAAGGAAAACGACAGAATCTTCGTTTCAGTCACC |
| | AACGAACATTTGATTGATATGGACCACGAAGCATCCTTTTTCGGTGCCTTTTTAGTAGGT |
| | GGAGGCGGTGGCAGTGGCGGAGGAGGCTCAGGCGGAGGTGGCAGTGTCAGAGAAAG |
| | AGGTCCACAAATAGTCGCCGCCCACATAACAGGTACAAGAGGTAGAAGTAACACATTAA |
| | GTTCCCCAAATAGTAAGAATGAAAAAGCTTTGGGTAGAAAGATTAACTCTTGGGAATCT |
| | TCAAGATCCGGTCATTCATTTTTGTCTAATTTGCACTTAAGAAACGGTGAATTAGTCATTC |
| | ATGAAAAGGGTTTCTACTACATCTATTCTCAAACATACTTCAGATTCCAAGAAGAAATTA |
| | AAGAAAACACCAAAAACGATAAGCAAATGGTACAATACATCTATAAGTACACAAGTTAT |
| | CCAGACCCTATCTTGTTGATGAAGTCTGCAAGAAACTCATGTTGGTCCAAGGATGCCGAA |
| | TACGGTTTGTACTCTATCTATCAAGGTGGTGTCTTCGAATTGAAGGAAAACGACAGAATC |
| | TTCGTTTCAGTCACCAACGAACATTTGATTGATATGGACCACGAAGCATCCTTTTTCGGTG |
| | CCTTTTTAGTAGGT |
| T151 | ATGGCCTGGCGGCTGTGGTGGCTGCTGCTCCTGCTCCTGTTGCTTTGGCCTATGGTGTGG |
| (SEQ ID NO: 52) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTCAGAGAAAGAGGTCCACAAAGAGTCGCCGCCCACA |
| | TAACAGGTACAAGAGGTAGAAGTAACACATTAAGTTCCCCAAATAGTAAGAATGAAAAA |
| | GCTTTGGGTAGAAAGATTAACTCTTGGGAATCTTCAAGATCCGGTCATTCATTTTTGTCTA |
| | ATTTGCACTTAAGAAACGGTGAATTAGTCATTCATGAAAAGGGTTTCTACTACATCTATTC |
| | TCAAACATACTTCAGATTCCAAGAAGAAATTAAAGAAAACACCAAAAACGATAAGCAAA |
| | TGGTACAATACATCTATAAGTACACAAGTTATCCAGACCCTATCTTGTTGATGAAGTCTG |
| | CAAGAAGCTCATGTTGGTCCAAGGATGCCGAATACGGTTTGTACTCTATCTATCAAGGTG |
| | GTGTCTTCGAATTGAAGGAAAACGACAGAATCTTCGTTTCAGTCACCAACGAACATTTGA |
| | TTGATATGGACCACGAAGCATCCTTTTTCGGTGCCTTTTTAGTAGGTGGCGGTGGAGGAT |
| | CAGGAGGTGGCGGTAGTGGTGGCGGAGGTTCAGTCAGAGAAAGAGGTCCACAAAGAG |
| | TCGCCGCCCACATAACAGGTACAAGAGGTAGAAGTAACACATTAAGTTCCCCAAATAGT |
| | AAGAATGAAAAAGCTTTGGGTAGAAAGATTAACTCTTGGGAATCTTCAAGATCCGGTCA |
| | TTCATTTTTGTCTAATTTGCACTTAAGAAACGGTGAATTAGTCATTCATGAAAAGGGTTTC |
| | TACTACATCTATTCTCAAACATACTTCAGATTCCAAGAAGAAATTAAAGAAAACACCAAA |
| | AACGATAAGCAAATGGTACAATACATCTATAAGTACACAAGTTATCCAGACCCTATCTTG |
| | TTGATGAAGTCTGCAAGAAGCTCATGTTGGTCCAAGGATGCCGAATACGGTTTGTACTCT |
| | ATCTATCAAGGTGGTGTCTTCGAATTGAAGGAAAACGACAGAATCTTCGTTTCAGTCACC |
| | AACGAACATTTGATTGATATGGACCACGAAGCATCCTTTTTCGGTGCCTTTTTAGTAGGT |
| | GGAGGCGGTGGCAGTGGCGGAGGAGGCTCAGGCGGAGGTGGCAGTGTCAGAGAAAG |
| | AGGTCCACAAAGAGTCGCCGCCCACATAACAGGTACAAGAGGTAGAAGTAACACATTAA |
| | GTTCCCCAAATAGTAAGAATGAAAAAGCTTTGGGTAGAAAGATTAACTCTTGGGAATCT |
| | TCAAGATCCGGTCATTCATTTTTGTCTAATTTGCACTTAAGAAACGGTGAATTAGTCATTC |
| | ATGAAAAGGGTTTCTACTACATCTATTCTCAAACATACTTCAGATTCCAAGAAGAAATTA |
| | AAGAAAACACCAAAAACGATAAGCAAATGGTACAATACATCTATAAGTACACAAGTTAT |
| | CCAGACCCTATCTTGTTGATGAAGTCTGCAAGAAGCTCATGTTGGTCCAAGGATGCCGA |
| | ATACGGTTTGTACTCTATCTATCAAGGTGGTGTCTTCGAATTGAAGGAAAACGACAGAAT |
| | CTTCGTTTCAGTCACCAACGAACATTTGATTGATATGGACCACGAAGCATCCTTTTTCGGT |
| | GCCTTTTTAGTAGGT |
| T153 | ATGGCCTGGCGGCTGTGGTGGCTGCTGCTCCTGCTCCTGTTGCTTTGGCCTATGGTGTGG |
| (SEQ ID NO: 53) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTCAGAGAAAGAGGTCCACAAAGAGTCGCCGCCCACA |
| | TAACAGGTACAGGAGGTAGAAGTAACACATTAAGTTCCCCAAATAGTAAGAATGAAAAA |
| | GCTTTGGGTAGAAAGATTAACTCTTGGGAATCTTCAAGATCCGGTCATTCATTTTTGTCTA |
| | ATTTGCACTTAAGAAACGGTGAATTAGTCATTCATGAAAAGGGTTTCTACTACATCTATTC |
| | TCAAACATACTTCAGATTCCAAGAAGAAATTAAAGAAAACACCAAAAACGATAAGCAAA |
| | TGGTACAATACATCTATAAGTACACAAGTTATCCAGACCCTATCTTGTTGATGAAGTCTG |
| | CAAGAAACTCATGTTGGTCCAAGGATGCCGAATACGGTTTGTACTCTATCTATCAAGGTG |
| | GTGTCTTCGAATTGAAGGAAAACGACAGAATCTTCGTTTCAGTCACCAACGAACATTTGA |
| | TTGATATGGACCACGAAGCATCCTTTTTCGGTGCCTTTTTAGTAGGTGGCGGTGGAGGAT |
| | CAGGAGGTGGCGGTAGTGGTGGCGGAGGTTCAGTCAGAGAAAGAGGTCCACAAAGAG |
| | TCGCCGCCCACATAACAGGTACAGGAGGTAGAAGTAACACATTAAGTTCCCCAAATAGT |
| | AAGAATGAAAAAGCTTTGGGTAGAAAGATTAACTCTTGGGAATCTTCAAGATCCGGTCA |
| | TTCATTTTTGTCTAATTTGCACTTAAGAAACGGTGAATTAGTCATTCATGAAAAGGGTTTC |
| | TACTACATCTATTCTCAAACATACTTCAGATTCCAAGAAGAAATTAAAGAAAACACCAAA |
| | AACGATAAGCAAATGGTACAATACATCTATAAGTACACAAGTTATCCAGACCCTATCTTG |
| | TTGATGAAGTCTGCAAGAAACTCATGTTGGTCCAAGGATGCCGAATACGGTTTGTACTCT |
| | ATCTATCAAGGTGGTGTCTTCGAATTGAAGGAAAACGACAGAATCTTCGTTTCAGTCACC |
| | AACGAACATTTGATTGATATGGACCACGAAGCATCCTTTTTCGGTGCCTTTTTAGTAGGT |
| | GGAGGCGGTGGCAGTGGCGGAGGAGGCTCAGGCGGAGGTGGCAGTGTCAGAGAAAG |
| | AGGTCCACAAAGAGTCGCCGCCCACATAACAGGTACAGGAGGTAGAAGTAACACATTA |
| | AGTTCCCCAAATAGTAAGAATGAAAAAGCTTTGGGTAGAAAGATTAACTCTTGGGAATC |
| | TTCAAGATCCGGTCATTCATTTTTGTCTAATTTGCACTTAAGAAACGGTGAATTAGTCATT |
| | CATGAAAAGGGTTTCTACTACATCTATTCTCAAACATACTTCAGATTCCAAGAAGAAATT |
| | AAAGAAAACACCAAAAACGATAAGCAAATGGTACAATACATCTATAAGTACACAAGTTA |
| | TCCAGACCCTATCTTGTTGATGAAGTCTGCAAGAAACTCATGTTGGTCCAAGGATGCCGA |
| | ATACGGTTTGTACTCTATCTATCAAGGTGGTGTCTTCGAATTGAAGGAAAACGACAGAAT |
| | CTTCGTTTCAGTCACCAACGAACATTTGATTGATATGGACCACGAAGCATCCTTTTTCGGT |
| | GCCTTTTTAGTAGGT |
| T183 | ATGGCCTGGCGGCTGTGGTGGCTGCTGCTCCTGCTCCTGTTGCTTTGGCCTATGGTGTGG |
| (SEQ ID NO: 54) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATGGACCGACTACCCGGACCCGATCCTGCTGATGAAATC |
| | TGCGCGTAACAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGGGCGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATGGACCGACTACCCGGACCCG |
| | ATCCTGCTGATGAAATCTGCGCGTAACAGCTGCTGGTCTAAAGATGCCGAATACGGCCT |
| | GTATAGCATTTACCAGGGCGGTGTGTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATGG |
| | ACCGACTACCCGGACCCGATCCTGCTGATGAAATCTGCGCGTAACAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T186 | ATGGCCTGGCGGCTGTGGTGGCTGCTGCTCCTGCTCCTGTTGCTTTGGCCTATGGTGTGG |
| (SEQ ID NO: 55) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGCGTGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATGGACCGACTACCCGGACCCGATCCTGCTGATGAAATC |
| | TGCGCGTAACAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | CGTGTTGCAGCCCATATTACCGGCACGGGCGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATGGACCGACTACCCGGACCC |
| | GATCCTGCTGATGAAATCTGCGCGTAACAGCTGCTGGTCTAAAGATGCCGAATACGGCC |
| | TGTATAGCATTTACCAGGGCGGTGTGTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGCGTGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATGG |
| | ACCGACTACCCGGACCCGATCCTGCTGATGAAATCTGCGCGTAACAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T191 | ATGGCCTGGCGGCTGTGGTGGCTGCTGCTCCTGCTCCTGTTGCTTTGGCCTATGGTGTGG |
| (SEQ ID NO: 56) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGCGTGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATGGACCGACTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | CGTGTTGCAGCCCATATTACCGGCACGGGCGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATGGACCGACTACCCGGACCC |
| | GATCCTGCTGATGAAATCTGCGCGTAGCAGCTGCTGGTCTAAAGATGCCGAATACGGCC |
| | TGTATAGCATTTACCAGGGCGGTGTGTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGCGTGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATGG |
| | ACCGACTACCCGGACCCGATCCTGCTGATGAAATCTGCGCGTAGCAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T202 | ATGGCCTGGCGGCTGTGGTGGCTGCTGCTCCTGCTCCTGTTGCTTTGGCCTATGGTGTGG |
| (SEQ ID NO: 57) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGCGGGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | CGGGTTGCAGCCCATATTACCGGCACGGGCGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATACACCAGCTACCCGGACCC |
| | GATCCTGCTGATGAAATCTGCGCGTAGCAGCTGCTGGTCTAAAGATGCCGAATACGGCC |
| | TGTATAGCATTTACCAGGGCGGTGTGTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGCGGGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T203 | ATGGCCTGGCGGCTGTGGTGGCTGCTGCTCCTGCTCCTGTTGCTTTGGCCTATGGTGTGG |
| (SEQ ID NO: 58) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGGGCGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATACACCAGCTACCCGGACCCG |
| | ATCCTGCTGATGAAATCTGCGCGTAGCAGCTGCTGGTCTAAAGATGCCGAATACGGCCT |
| | GTATAGCATTTACCAGGGCGGTGTGTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T204 | ATGGCCTGGCGGCTGTGGTGGCTGCTGCTCCTGCTCCTGTTGCTTTGGCCTATGGTGTGG |
| (SEQ ID NO: 59) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGCGGGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAACAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGCGGGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATACACCAGCTACCCGGACCCG |
| | ATCCTGCTGATGAAATCTGCGCGTAACAGCTGCTGGTCTAAAGATGCCGAATACGGCCT |
| | GTATAGCATTTACCAGGGCGGTGTGTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGCGGGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAACAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T205 | ATGGCCTGGCGGCTGTGGTGGCTGCTGCTCCTGCTCCTGTTGCTTTGGCCTATGGTGTGG |
| (SEQ ID NO: 60) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGCGGGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAACAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | CGGGTTGCAGCCCATATTACCGGCACGGGCGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATACACCAGCTACCCGGACCC |
| | GATCCTGCTGATGAAATCTGCGCGTAACAGCTGCTGGTCTAAAGATGCCGAATACGGCC |
| | TGTATAGCATTTACCAGGGCGGTGTGTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGCGGGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAACAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T207 | ATGGCCTGGCGGCTGTGGTGGCTGCTGCTCCTGCTCCTGTTGCTTTGGCCTATGGTGTGG |
| (SEQ ID NO: 61) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGAGGGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTGTGTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGAGGGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATACACCAGCTACCCGGACCC |
| | GATCCTGCTGATGAAATCTGCGCGTAGCAGCTGCTGGTCTAAAGATGCCGAATACGGCC |
| | TGTATAGCATTTACCAGGGCGGTGTGTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGAGGGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTGTGTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T208 | ATGGCCTGGCGGCTGTGGTGGCTGCTGCTCCTGCTCCTGTTGCTTTGGCCTATGGTGTGG |
| (SEQ ID NO: 62) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAACAGCTGCTGGTCTAAAGATGCCGAATACGGCCTGTATAGCATTTACCAGG |
| | GCGGTATCTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGGGCGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATACACCAGCTACCCGGACCCG |
| | ATCCTGCTGATGAAATCTGCGCGTAACAGCTGCTGGTCTAAAGATGCCGAATACGGCCT |
| | GTATAGCATTTACCAGGGCGGTATCTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAACAGCTGCTGGTCTAA |
| | AGATGCCGAATACGGCCTGTATAGCATTTACCAGGGCGGTATCTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T209 | ATGGCCTGGCGGCTGTGGTGGCTGCTGCTCCTGCTCCTGTTGCTTTGGCCTATGGTGTGG |
| (SEQ ID NO: 63) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTATCTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGGGCGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATACACCAGCTACCCGGACCCG |
| | ATCCTGCTGATGAAATCTGCGCGTAGCAGCTGCTGGTCTAAAGATGCCGAATACGGCCT |
| | GTATAGCATTTACCAGGGCGGTATCTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTATCTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T210 | ATGGCCTGGCGGCTGTGGTGGCTGCTGCTCCTGCTCCTGTTGCTTTGGCCTATGGTGTGG |
| (SEQ ID NO: 64) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGATCGTTGCAGCCCATA |
| | TTACCGGCACGCGGGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTATCTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | ATCGTTGCAGCCCATATTACCGGCACGCGGGGTCGCTCTAACACGCTGAGCTCTCCGAAC |
| | AGTAAAAATGAAAAAGCGCTGGGCCGTAAAATCAATTCTTGGGAAAGTAGCCGCAGCG |
| | GTCATTCTTTTCTGAGTAACCTGCACCTGCGTAATGGCGAACTGGTGATCCACGAAAAAG |
| | GTTTCTACTACATCTACAGCCAGACCTACTTTCGCTTCCAGGAAGAAATCAAAGAAAACA |
| | CGAAAAACGATAAACAGATGGTTCAGTACATCTACAAATACACCAGCTACCCGGACCCG |
| | ATCCTGCTGATGAAATCTGCGCGTAGCAGCTGCTGGTCTAAAGATGCCGAATACGGCCT |
| | GTATAGCATTTACCAGGGCGGTATCTTTGAACTGAAAGAAAACGATCGCATTTTCGTGTC |
| | TGTTACCAATGAACATCTGATCGATATGGATCACGAAGCGAGCTTTTTCGGTGCCTTTCT |
| | GGTGGGCGGCGGGGGCGGCAGTGGTGGAGGTGGATCTGGCGGAGGAGGCTCTGTGC |
| | GTGAACGTGGTCCGCAGATCGTTGCAGCCCATATTACCGGCACGCGGGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTATCTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |
| T211 | ATGGCCTGGCGGCTGTGGTGGCTGCTGCTCCTGCTCCTGTTGCTTTGGCCTATGGTGTGG |
| (SEQ ID NO: 65) | GCTGATAAGACACATACATGCCCTCCATGTCCCGCACCCGAGTTGCTTGGAGGACCTTCG |
| | GTGTTTCTTTTTCCCCCGAAGCCAAAAGATACACTGATGATTTCACGGACGCCCGAGGTG |
| | ACTTGTGTCGTCGTGGACGTCAGCCACGAGGACCCAGAAGTCAAGTTTAACTGGTATGT |
| | AGATGGGGTGGAGGTACACAATGCGAAAACGAAACCGAGAGAGGAGCAGTACAATTC |
| | GACGTATAGGGTGGTCAGCGTGCTGACGGTGTTGCACCAGGACTGGCTGAACGGGAAA |
| | GAGTATAAGTGCAAAGTGTCGAACAAGGCCCTCCCCGCACCCATCGAAAAGACGATATC |
| | CAAAGCCAAGGGCCAACCGCGCGAGCCGCAAGTGTACACGCTGCCTCCCTCGCGAGAA |
| | GAGATGACCAAGAACCAGGTGTCCCTTACGTGCTTGGTGAAAGGATTCTACCCTTCGGA |
| | CATCGCCGTAGAATGGGAAAGCAATGGGCAGCCAGAGAACAATTACAAAACCACACCG |
| | CCTGTGCTCGACTCGGACGGTTCCTTTTTCTTGTATTCCAAGTTGACAGTGGACAAGTCA |
| | CGGTGGCAACAGGGGAACGTATTCTCGTGTTCCGTCATGCACGAAGCGCTGCATAACCA |
| | CTACACTCAGAAGTCGCTAAGCTTGTCGCCGGGTGGAGGAGGCGGCAGTGGTGGAGGT |
| | GGATCTGGCGGAGGAGGCTCTGTGCGTGAACGTGGTCCGCAGAGGGTTGCAGCCCATA |
| | TTACCGGCACGGGCGGTCGCTCTAACACGCTGAGCTCTCCGAACAGTAAAAATGAAAAA |
| | GCGCTGGGCCGTAAAATCAATTCTTGGGAAAGTAGCCGCAGCGGTCATTCTTTTCTGAGT |
| | AACCTGCACCTGCGTAATGGCGAACTGGTGATCCACGAAAAAGGTTTCTACTACATCTAC |
| | AGCCAGACCTACTTTCGCTTCCAGGAAGAAATCAAAGAAAACACGAAAAACGATAAACA |
| | GATGGTTCAGTACATCTACAAATACACCAGCTACCCGGACCCGATCCTGCTGATGAAATC |
| | TGCGCGTAGCAGCTGCTGGTCTAAAGATGCCGAATACGGCCTGTATAGCATTTACCAGG |
| | GCGGTATCTTTGAACTGAAAGAAAACGATCGCATTTTCGTGTCTGTTACCAATGAACATC |
| | TGATCGATATGGATCACGAAGCGAGCTTTTTCGGTGCCTTTCTGGTGGGCGGTGGTGGC |
| | GGCAGTGGTGGAGGTGGATCTGGCGGAGGAGGCTCTGTGCGTGAACGTGGTCCGCAG |
| | AGGGTTGCAGCCCATATTACCGGCACGGGCGGTCGCTCTAACACGCTGAGCTCTCCGAA |
| | CAGTAAAAATGAAAAAGCGCTGGGCCGTAAAATCAATTCTTGGGAAAGTAGCCGCAGC |
| | GGTCATTCTTTTCTGAGTAACCTGCACCTGCGTAATGGCGAACTGGTGATCCACGAAAAA |
| | GGTTTCTACTACATCTACAGCCAGACCTACTTTCGCTTCCAGGAAGAAATCAAAGAAAAC |
| | ACGAAAAACGATAAACAGATGGTTCAGTACATCTACAAATACACCAGCTACCCGGACCC |
| | GATCCTGCTGATGAAATCTGCGCGTAGCAGCTGCTGGTCTAAAGATGCCGAATACGGCC |
| | TGTATAGCATTTACCAGGGCGGTATCTTTGAACTGAAAGAAAACGATCGCATTTTCGTGT |
| | CTGTTACCAATGAACATCTGATCGATATGGATCACGAAGCGAGCTTTTTCGGTGCCTTTC |
| | TGGTGGGCGGCGGGGGCGGCAGTGGTGGAGGTGGATCTGGCGGAGGAGGCTCTGTGC |
| | GTGAACGTGGTCCGCAGAGGGTTGCAGCCCATATTACCGGCACGGGCGGTCGCTCTAAC |
| | ACGCTGAGCTCTCCGAACAGTAAAAATGAAAAAGCGCTGGGCCGTAAAATCAATTCTTG |
| | GGAAAGTAGCCGCAGCGGTCATTCTTTTCTGAGTAACCTGCACCTGCGTAATGGCGAAC |
| | TGGTGATCCACGAAAAAGGTTTCTACTACATCTACAGCCAGACCTACTTTCGCTTCCAGG |
| | AAGAAATCAAAGAAAACACGAAAAACGATAAACAGATGGTTCAGTACATCTACAAATAC |
| | ACCAGCTACCCGGACCCGATCCTGCTGATGAAATCTGCGCGTAGCAGCTGCTGGTCTAA |
| | AGATGCCGAATACGGCCTGTATAGCATTTACCAGGGCGGTATCTTTGAACTGAAAGAAA |
| | ACGATCGCATTTTCGTGTCTGTTACCAATGAACATCTGATCGATATGGATCACGAAGCGA |
| | GCTTTTTCGGTGCCTTTCTGGTGGGC |

TABLE 11
Mutant TRAIL trimer polypeptide sequences without an
Fc region sequence.

| Variant | Amino Acid Sequence |
|---|---|
| T148 - without Fc | VRERGPQIVAAHITGTRGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 66) | VQYIYKYTSYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQIVAAHITGTRGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARNSCWSKDAEYGLYSIYQGGVFELKEN |
| | DRIFVSVTNEHLIDMDHEASFFGAFLVGGGGGSGGGGSGGGGSVRE |
| | RGPQIVAAHITGTRGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARNSCWSKDAEYGLYSIYQGGVFELKENDRI |
| | FVSVTNEHLIDMDHEASFFGAFLVG |
| T151 - without Fc | VRERGPQRVAAHITGTRGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 67) | MVQYIYKYTSYPDPILLMKSARSSCWSKDAEYGLYSIYQGGVFELK |
| | ENDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVG |
| T153 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 68) | MVQYIYKYTSYPDPILLMKSARNSCWSKDAEYGLYSIYQGGVFELK |
| | ENDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARNSCWSKDAEYGLYSIYQGGVFELKE |
| | NDRIFVSVTNEHLIDMDHEASFFGAFLVG |
| T183 - without Fc | VRERGPQIVAAHITGTGGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 69) | VQYIYKWTDYPDPILLMKSARNSCWSKDAEYGLYSIYQGGVFELK |
| | ENDRIFVSVTNEHLIDMDHEASFFGAFLVGGGGGSGGGGSGGGGSV |
| | RERGPQIVAAHITGTGGRSNTLSSPNSKNEKALGRKINSWESSRSGH |
| | SFLSNLHLRNGELVIHEKGFYYIYSQTYFRFQEEIKENTKNDKQMV |
| | QYIYKWTDYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQIVAAHITGTGGRSNTLSSPNSKNEKALGRKINSWESSRSGH |
| | SFLSNLHLRNGELVIHEKGFYYIYSQTYFRFQEEIKENTKNDKQMV |
| | QYIYKWTDYPDPILLMKSARNSCWSKDAEYGLYSIYQGGVFELKE |
| | NDRIFVSVTNEHLIDMDHEASFFGAFLVG |
| T186 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 70) | MVQYIYKWTDYPDPILLMKSARNSCWSKDAEYGLYSIYQGGVFEL |
| | KENDRIFVSVTNEHLIDMDHEASFFGAFLVGGGGGSGGGGSGGGGS |
| | VRERGPQRVAAHITGTGGRSNTLSSPNSKNEKALGRKINSWESSRS |
| | GHSFLSNLHLRNGELVIHEKGFYYIYSQTYFRFQEEIKENTKNDKQ |
| | MVQYIYKWTDYPDPILLMKSARNSCWSKDAEYGLYSIYQGGVFEL |
| | KENDRIFVSVTNEHLIDMDHEASFFGAFLVGGGGGSGGGGSGGGGS |
| | VRERGPQRVAAHITGTGGRSNTLSSPNSKNEKALGRKINSWESSRS |
| | GHSFLSNLHLRNGELVIHEKGFYYIYSQTYFRFQEEIKENTKNDKQ |
| | MVQYIYKWTDYPDPILLMKSARNSCWSKDAEYGLYSIYQGGVFEL |
| | KENDRIFVSVTNEHLIDMDHEASFFGAFLVG |
| T191 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 71) | MVQYIYKWTDYPDPILLMKSARSSCWSKDAEYGLYSIYQGGVFEL |
| | KENDRIFVSVTNEHLIDMDHEASFFGAFLVGGGGGSGGGGSGGGGS |
| | VRERGPQRVAAHITGTGGRSNTLSSPNSKNEKALGRKINSWESSRS |
| | GHSFLSNLHLRNGELVIHEKGFYYIYSQTYFRFQEEIKENTKNDKQ |
| | MVQYIYKWTDYPDPILLMKSARSSCWSKDAEYGLYSIYQGGVFEL |
| | KENDRIFVSVTNEHLIDMDHEASFFGAFLVGGGGGSGGGGSGGGGS |
| | VRERGPQRVAAHITGTGGRSNTLSSPNSKNEKALGRKINSWESSRS |
| | GHSFLSNLHLRNGELVIHEKGFYYIYSQTYFRFQEEIKENTKNDKQ |
| | MVQYIYKWTDYPDPILLMKSARSSCWSKDAEYGLYSIYQGGVFEL |
| | KENDRIFVSVTNEHLIDMDHEASFFGAFLVG |
| T202 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 72) | MVQYIYKYTSYPDPILLMKSARSSCWSKDAEYGLYSIYQGGVFELK |
| | ENDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVG |
| T203 - without Fc | VRERGPQIVAAHITGTGGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 73) | VQYIYKYTSYPDPILLMKSARSSCWSKDAEYGLYSIYQGGVFELKE |
| | NDRIFVSVTNEHLIDMDHEASFFGAFLVGGGGGSGGGGSGGGGSV |
| | RERGPQIVAAHITGTGGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARSSCWSKDAEYGLYSIYQGGVFELKEN |
| | DRIFVSVTNEHLIDMDHEASFFGAFLVGGGGGSGGGGSGGGGSVRE |
| | RGPQIVAAHITGTGGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARSSCWSKDAEYGLYSIYQGGVFELKENDRI |
| | FVSVTNEHLIDMDHEASFFGAFLVG |
| T204 - without Fc | VRERGPQIVAAHITGTRGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 74) | VQYIYKYTSYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQIVAAHITGTRGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARNSCWSKDAEYGLYSIYQGGVFELKEN |
| | DRIFVSVTNEHLIDMDHEASFFGAFLVGGGGGSGGGGSGGGGSVRE |
| | RGPQIVAAHITGTRGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARNSCWSKDAEYGLYSIYQGGVFELKENDRI |
| | FVSVTNEHLIDMDHEASFFGAFLVG |
| T205 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 75) | MVQYIYKYTSYPDPILLMKSARNSCWSKDAEYGLYSIYQGGVFELK |
| | ENDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARNSCWSKDAEYGLYSIYQGGVFELKE |
| | NDRIFVSVTNEHLIDMDHEASFFGAFLVG |
| T206 - without Fc | VRERGPQRVAAHITGTRGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 76) | MVQYIYKYTSYPDPILLMKSARSSCWSKDAEYGLYSIYQGGVFELK |
| | ENDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGVFELKE |
| | NDRIFVSVTNEHLIDMDHEASFFGAFLVG |
| T207 - without Fc | VRERGPQIVAAHITGTRGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 77) | VQYIYKYTSYPDPILLMKSARSSCWSKDAEYGLYSIYQGGVFELKE |
| | NDRIFVSVTNEHLIDMDHEASFFGAFLVGGGGGSGGGGSGGGGSV |
| | RERGPQIVAAHITGTRGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARSSCWSKDAEYGLYSIYQGGVFELKEN |
| | DRIFVSVTNEHLIDMDHEASFFGAFLVGGGGGSGGGGSGGGGSVRE |
| | RGPQIVAAHITGTRGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARSSCWSKDAEYGLYSIYQGGVFELKENDRI |
| | FVSVTNEHLIDMDHEASFFGAFLVG |
| T208 - without Fc | VRERGPQIVAAHITGTGGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 78) | VQYIYKYTSYPDPILLMKSARNSCWSKDAEYGLYSIYQGGIFELKE |
| | NDRIFVSVTNEHLIDMDHEASFFGAFLVGGGGGSGGGGSGGGGSV |
| | RERGPQIVAAHITGTGGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARNSCWSKDAEYGLYSIYQGGIFELKEN |
| | DRIFVSVTNEHLIDMDHEASFFGAFLVGGGGGSGGGGSGGGGSVRE |
| | RGPQIVAAHITGTGGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARNSCWSKDAEYGLYSIYQGGIFELKENDRIF |
| | VSVTNEHLIDMDHEASFFGAFLVG |
| T209 - without Fc | VRERGPQIVAAHITGTGGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 79) | VQYIYKYTSYPDPILLMKSARSSCWSKDAEYGLYSIYQGGIFELKEN |
| | DRIFVSVTNEHLIDMDHEASFFGAFLVGGGGGSGGGGSGGGGSVRE |
| | RGPQIVAAHITGTGGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARSSCWSKDAEYGLYSIYQGGIFELKENDRIF |
| | VSVTNEHLIDMDHEASFFGAFLVGGGGGSGGGGSGGGGSVRERGP |
| | QIVAAHITGTGGRSNTLSSPNSKNEKALGRKINSWESSRSGHSFLSN |
| | LHLRNGELVIHEKGFYYIYSQTYFRFQEEIKENTKNDKQMVQYIYK |
| | YTSYPDPILLMKSARSSCWSKDAEYGLYSIYQGGIFELKENDRIFVS |
| | VTNEHLIDMDHEASFFGAFLVG |
| T210 - without Fc | VRERGPQIVAAHITGTRGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 80) | VQYIYKYTSYPDPILLMKSARSSCWSKDAEYGLYSIYQGGIFELKEN |
| | DRIFVSVTNEHLIDMDHEASFFGAFLVGGGGGSGGGGSGGGGSVRE |
| | RGPQIVAAHITGTRGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARSSCWSKDAEYGLYSIYQGGIFELKENDRIF |
| | VSVTNEHLIDMDHEASFFGAFLVGGGGGSGGGGSGGGGSVRERGP |
| | QIVAAHITGTRGRSNTLSSPNSKNEKALGRKINSWESSRSGHSFLSN |
| | LHLRNGELVIHEKGFYYIYSQTYFRFQEEIKENTKNDKQMVQYIYK |
| | YTSYPDPILLMKSARSSCWSKDAEYGLYSIYQGGIFELKENDRIFVS |
| | VTNEHLIDMDHEASFFGAFLVG |
| T211 - without Fc | VRERGPQRVAAHITGTGGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 81) | MVQYIYKYTSYPDPILLMKSARSSCWSKDAEYGLYSIYQGGIFELK |
| | ENDRIFVSVTNEHLIDMDHEASFFGAFLVGGGGGSGGGGSGGGGSV |
| | RERGPQRVAAHITGTGGRSNTLSSPNSKNEKALGRKINSWESSRSG |
| | HSFLSNLHLRNGELVIHEKGFYYIYSQTYFRFQEEIKENTKNDKQM |
| | VQYIYKYTSYPDPILLMKSARSSCWSKDAEYGLYSIYQGGIFELKEN |
| | DRIFVSVTNEHLIDMDHEASFFGAFLVGGGGGSGGGGSGGGGSVRE |
| | RGPQRVAAHITGTGGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARSSCWSKDAEYGLYSIYQGGIFELKENDRIF |
| | VSVTNEHLIDMDHEASFFGAFLVG |
| T182 - without Fc | VRERGPQRVAAHITGTRGRSNTLSSPNSKNEKALGRKINSWESSRS |
| region | GHSFLSNLHLRNGELVIHEKGFYYIYSQTYFRFQEEIKENTKNDKQ |
| (SEQ ID NO: 102) | MVQYIYKWTDYPDPILLMKSARNSCWSKDAEYGLYSIYQGGIFELK |
| | ENDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKWTDYPDPILLMKSARNSCWSKDAEYGLYSIYQGGIFELKE |
| | NDRIFVSVTNEHLIDMDHEASFFGAFLVGGGGGSGGGGSGGGGSV |
| | RERGPQRVAAHITGTRGRSNTLSSPNSKNEKALGRKINSWESSRSG |
| | HSFLSNLHLRNGELVIHEKGFYYIYSQTYFRFQEEIKENTKNDKQM |
| | VQYIYKWTDYPDPILLMKSARNSCWSKDAEYGLYSIYQGGIFELKE |
| | NDRIFVSVTNEHLIDMDHEASFFGAFLVG |
| T196 - without Fc | VRERGPQIVAAHITGTGGRSNTLSSPNSKNEKALGRKINSWESSRSG |
| region | HSFLSNLHLRNGELVIHEKGFYYIYSQTYFRFQEEIKENTKNDKQM |
| (SEQ ID NO: 103) | VQYIYKYTSYPDPILLMKSARNSCWSKDAEYGLYSIYQGGVFELKE |
| | NDRIFVSVTNEHLIDMDHEASFFGAFLVGGGGGSGGGGSGGGGSV |
| | RERGPQIVAAHITGTGGRSNTLSSPNSKNEKALGRKINSWESSRSGH |
| | SFLSNLHLRNGELVIHEKGFYYIYSQTYFRFQEEIKENTKNDKQMV |
| | QYIYKYTSYPDPILLMKSARNSCWSKDAEYGLYSIYQGGVFELKEN |
| | DRIFVSVTNEHLIDMDHEASFFGAFLVGGGGGSGGGGSGGGGSVRE |
| | RGPQIVAAHITGTGGRSNTLSSPNSKNEKALGRKINSWESSRSGHSF |
| | LSNLHLRNGELVIHEKGFYYIYSQTYFRFQEEIKENTKNDKQMVQYI |
| | YKYTSYPDPILLMKSARNSCWSKDAEYGLYSIYQGGVFELKENDRI |
| | FVSVTNEHLIDMDHEASFFGAFLVG |

TABLE 12
Mutant TRAIL monomer sequences.

| Variant | Amino Acid Sequence |
|---|---|
| T148 - TRAIL | VRERGPQIVAAHITGTRGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 82) | VQYIYKYTSYPDPILLMKSARNSCWSKDAEYGLYSIYQGGVFELKE |
| | NDRIFVSVTNEHLIDMDHEASFFGAFLVG |
| T151 - TRAIL | VRERGPQRVAAHITGTRGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 83) | MVQYIYKYTSYPDPILLMKSARSSCWSKDAEYGLYSIYQGGVFELK |
| | ENDRIFVSVTNEHLIDMDHEASFFGAFLVG |
| T153 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 84) | MVQYIYKYTSYPDPILLMKSARNSCWSKDAEYGLYSIYQGGVFELK |
| | ENDRIFVSVTNEHLIDMDHEASFFGAFLVG |
| T183 - TRAIL | VRERGPQIVAAHITGTGGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 85) | VQYIYKWTDYPDPILLMKSARNSCWSKDAEYGLYSIYQGGVFELK |
| | ENDRIFVSVTNEHLIDMDHEASFFGAFLVG |
| T186 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 86) | MVQYIYKWTDYPDPILLMKSARNSCWSKDAEYGLYSIYQGGVFEL |
| | KENDRIFVSVTNEHLIDMDHEASFFGAFLVG |
| T191 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 87) | MVQYIYKWTDYPDPILLMKSARSSCWSKDAEYGLYSIYQGGVFEL |
| | KENDRIFVSVTNEHLIDMDHEASFFGAFLVG |
| T202 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 88) | MVQYIYKYTSYPDPILLMKSARSSCWSKDAEYGLYSIYQGGVFELK |
| | ENDRIFVSVTNEHLIDMDHEASFFGAFLVG |
| T203 - TRAIL | VRERGPQIVAAHITGTGGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 89) | VQYIYKYTSYPDPILLMKSARSSCWSKDAEYGLYSIYQGGVFELKE |
| | NDRIFVSVTNEHLIDMDHEASFFGAFLVG |
| T204 - TRAIL | VRERGPQIVAAHITGTRGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 90) | VQYIYKYTSYPDPILLMKSARNSCWSKDAEYGLYSIYQGGVFELKE |
| | NDRIFVSVTNEHLIDMDHEASFFGAFLVG |
| T205 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 91) | MVQYIYKYTSYPDPILLMKSARNSCWSKDAEYGLYSIYQGGVFELK |
| | ENDRIFVSVTNEHLIDMDHEASFFGAFLVG |
| T206 - TRAIL | VRERGPQRVAAHITGTRGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 92) | MVQYIYKYTSYPDPILLMKSARSSCWSKDAEYGLYSIYQGGVFELK |
| | ENDRIFVSVTNEHLIDMDHEASFFGAFLVG |
| T207 - TRAIL | VRERGPQIVAAHITGTRGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 93) | VQYIYKYTSYPDPILLMKSARSSCWSKDAEYGLYSIYQGGVFELKE |
| | NDRIFVSVTNEHLIDMDHEASFFGAFLVG |
| T208 - TRAIL | VRERGPQIVAAHITGTGGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 94) | VQYIYKYTSYPDPILLMKSARNSCWSKDAEYGLYSIYQGGIFELKE |
| | NDRIFVSVTNEHLIDMDHEASFFGAFLVG |
| T209 - TRAIL | VRERGPQIVAAHITGTGGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 95) | VQYIYKYTSYPDPILLMKSARSSCWSKDAEYGLYSIYQGGIFELKEN |
| | DRIFVSVTNEHLIDMDHEASFFGAFLVG |
| T210 - TRAIL | VRERGPQIVAAHITGTRGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 96) | VQYIYKYTSYPDPILLMKSARSSCWSKDAEYGLYSIYQGGIFELKEN |
| | DRIFVSVTNEHLIDMDHEASFFGAFLVG |
| T211 - TRAIL | VRERGPQRVAAHITGTGGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 97) | MVQYIYKYTSYPDPILLMKSARSSCWSKDAEYGLYSIYQGGIFELK |
| | ENDRIFVSVTNEHLIDMDHEASFFGAFLVG |
| T182 - TRAIL | VRERGPQRVAAHITGTRGRSNTLSSPNSKNEKALGRKINSWESSRS |
| monomer | GHSFLSNLHLRNGELVIHEKGFYYIYSQTYFRFQEEIKENTKNDKQ |
| (SEQ ID NO: 104) | MVQYIYKWTDYPDPILLMKSARNSCWSKDAEYGLYSIYQGGIFELK |
| | ENDRIFVSVTNEHLIDMDHEASFFGAFLVG |
| T196 - TRAIL | VRERGPQIVAAHITGTGGRSNTLSSPNSKNEKALGRKINSWESSRSG |
| monomer | HSFLSNLHLRNGELVIHEKGFYYIYSQTYFRFQEEIKENTKNDKQM |
| (SEQ ID NO: 105) | VQYIYKYTSYPDPILLMKSARNSCWSKDAEYGLYSIYQGGVFELKE |
| | NDRIFVSVTNEHLIDMDHEASFFGAFLVG |

### Example 14: Development of an Anti-EpcAM IgG-Sctrail Fusion Protein

### Methods

The heavy chain of MOC31 IgG (anti-EpCAM) fused to scTRAIL (SEQ ID NO: 98) was codon optimized for HEK293 expression, synthesized and cloned into the vector pCEP4 (Genscript, N.J.) using KpnI and NotI sites to create the plasmid pCEP4-MOC31 HC-scTRAIL. Underlined sequence represents the leader peptide.

SEQ ID NO: 98:
MGTPAQLLFLLLLWLPDTTGEVQLVQSGPGLVQPGGSVRISCAASGYTFT
NYGMNWVKQAPGKGLEWMGWINTYTGESTYADSFKGRFTFSLDTSASAAY
LQINSLRAEDTAVYYCARFAIKGDYWGQGTLLTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGGGGGSGGGGSGGGGSSVRERGPQRVAAHITGTRGR
SNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWS
KDAEYGLYSIYQGGIFELKENDRIFVSVTNEHLIDMDHEASFFGAFLVGG
GGGSGGGGSGGGGSVRERGPQRVAAHITGTRGRSNTLSSPNSKNEKALGR
KINSWESSRSGHSFLSNLHLRNGELVIHEKGFYYIYSQTYFRFQEEIKEN
TKNDKQMVQYIYKYTSYPDPILLMKSARNSCWSKDAEYGLYSIYQGGIFE
LKENDRIFVSVTNEHLIDMDHEASFFGAFLVGGGGGSGGGGSGGGGSVRE
RGPQRVAAHITGTRGRSNTLSSPNSKNEKALGRKINSWESSRSGHSFLSN
LHLRNGELVIHEKGFYYIYSQTYFRFQEEIKENTKNDKQMVQYIYKYTSY
PDPILLMKSARNSCWSKDAEYGLYSIYQGGIFELKENDRIFVSVTNEHLI
DMDHEASFFGAFLVG
SEQ ID NO: 99 is the mature anti-EpCAM IgG-scTRAIL
heavy chain fusion without the leader sequence.
SEQ ID NO: 99
EVQLVQSGPGLVQPGGSVRISCAASGYTFTNYGMNWVKQAPGKGLEWMGW
INTYTGESTYADSFKGRFTFSLDTSASAAYLQINSLRAEDTAVYYCARFA
IKGDYWGQGTLLTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGGGGGS
GGGGSGGGGSSVRERGPQRVAAHITGTRGRSNTLSSPNSKNEKALGRKIN
SWESSRSGHSFLSNLHLRNGELVIHEKGFYYIYSQTYFRFQEEIKENTKN
DKQMVQYIYKYTSYPDPILLMKSARNSCWSKDAEYGLYSIYQGGIFELKE
NDRIFVSVTNEHLIDMDHEASFFGAFLVGGGGGSGGGGSGGGGSVRERGP
QRVAAHITGTRGRSNTLSSPNSKNEKALGRKINSWESSRSGHSFLSNLHL
RNGELVIHEKGFYYIYSQTYFRFQEEIKENTKNDKQMVQYIYKYTSYPDP
ILLMKSARNSCWSKDAEYGLYSIYQGGIFELKENDRIFVSVTNEHLIDMD
HEASFFGAFLVGGGGGSGGGGSGGGGSVRERGPQRVAAHITGTRGRSNTL
SSPNSKNEKALGRKINSWESSRSGHSFLSNLHLRNGELVIHEKGFYYIYS
QTYFRFQEEIKENTKNDKQMVQYIYKYTSYPDPILLMKSARNSCWSKDAE
YGLYSIYQGGIFELKENDRIFVSVTNEHLIDMDHEASFFGAFLVG

HEK-293F cells (FREESTYLE HEK-293 cells adapted for suspension culture, ThermoFisher (Cat.#R79007) stably expressing the anti-apoptotic protein Bcl-XL are grown in FREESTYLE F17 media (Gibco) containing 4 mM L-glutamine (Gibco) and 1% PLURONIC F-68 (Gibco) as suspension cultures in flasks with rotation (125 rpm). Cells were co-transfected with a 0.5 μg of pCEP4-MOC31 heavy chain-scTRAIL and 0.5 μg of pCEP4-MOC31 light chain (1 μg of total DNA), and 2.5 μg of linear 25 kDa polyethylenimine (Polysciences Inc.) per milliliter of cell culture. Density of cells at time of transfection is 1.5-2.0 e6 cells/ml. Cells are fed the following day with Tryptone N1 (Organotechnie) added to a final concentration of 5 mg/ml. Six days post transfection, cell cultures are centrifuged for 15 min at 5,000×g to pellet the cells. The supernatant media are decanted from the cells and filtered using 0.2 μm filter in preparation for purification.

### Protein Purification

Media containing the MOC-31 IgG-scTRAIL was loaded onto MABSELECT (GE Heathcare) resin using an AKTA Explorer (Amersham Biosciences). Following affinity capture, the resin is washed with phosphate buffered saline (PBS), pH 7.4 (Gibco) and eluted with 0.1 M glycine-HCl, pH 3.5. The acid eluate is rapidly neutralized using 1:100 volume of 1 M Tris base. Proteins are dialyzed into PBS, pH 7.4 overnight and aliquoted the next day for storage at −80° C.

### Luminescent Cell Viability and Caspase 8 Activity Assay

Cells are seeded at 10,000 cells per well in 96 well tissue culture plate. Twenty-four hours later cells are incubated with increasing concentrations of either TRAIL, Fc-scTRAIL, or MOC31 IgG-scTRAIL proteins for either 0.5, 1, 2, 4, 8 or 24 hours. Post treatment, the cell viability was determined by measuring the amount of cellular ATP using CELLTITER-GLO Assay (Promega). Active caspase 8 levels was determined using Caspase-Glo 8 Assay (Promega). Luminescence was measured on a SYNERGY H1 plate reader (BioTek) and normalized to untreated controls and plotted as a function of protein concentration or time. Non-linear regression was fitted using a 4 parameter least square fit using PRISM software (GraphPad). Individual measurements luminescence measurements of the CELLTITER-GLO assay were also visualized in a heat map using MATLAB (The Mathworks, Inc.).

### Results

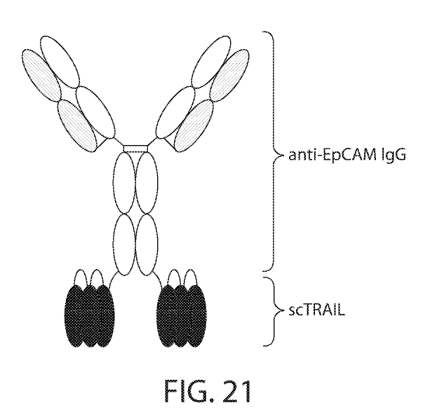

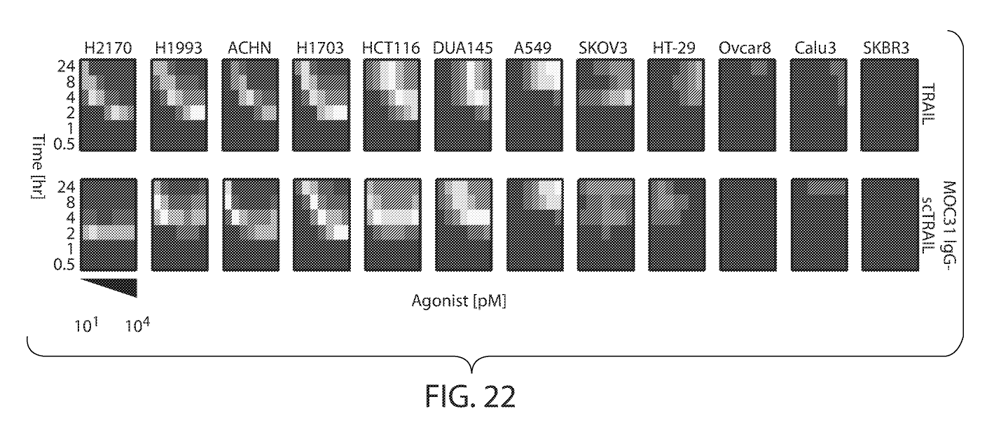

To determine whether binding of a tumor associated antigen could increase cell surface clustering of scTRAIL and lead to a greater induction in apoptosis, a tumor antigen antibody-scTRAIL fusion protein was developed. As shown in FIG. 21, MOC-31 IgG-scTRAIL consists of the anti-EpCAM antibody MOC-31 fused to the N-terminus of scTRAIL. To assess the activity of MOC-31 IgG-scTRAIL, a panel of cancer cell lines with low (ACHN, H1703, A549, and OVCAR8) or high (H2170, H1993, HCT116, DU145 SKOV3, HT29, CALU3 and SKBR3) EpCAM levels were treated with concentration range (0.005-10 nM) of native TRAIL or MOC-31 IgG-scTRAIL for 0.5, 1, 2, 4, 8 and 24 hours. Cell viability was assessed using a Cell Titer Glo assay and visualized in a heat map as a function of time and protein concentration (FIG. 22).

Among the cell lines tested, there was no positive correlation between EpCAM expression and TRAIL sensitivity. Cells that were intrinsically resistant to TRAIL induced apoptosis (A549, SKOV3, HT-29, OVCAR8, CALU3, and SKRR3) were also resistant to MOC-31 IgG-scTRAIL regardless of EpCAM levels. Thus, the presence of the EpCAM binding antibody did not infer TRAIL sensitivity.

Binding EpCAM did increase potency in TRAIL sensitive cells (H2170, H1993, ACHN, H1703, HCT116 and DU145). This was reflected in a lower IC₅₀ for MOC-31 IgG-scTRAIL compared to TRAIL. However, the maximum number of cells that underwent apoptosis did not increase with EpCAM binding. To accurately monitor the time dependency of apoptosis for

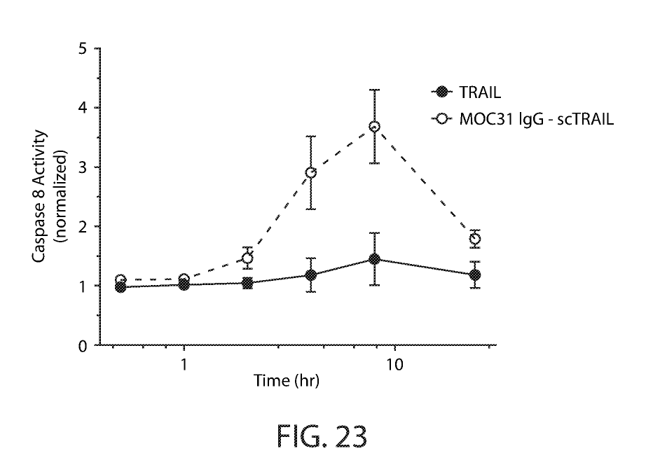

MOC IgG-scTRAIL, caspase 8 activation was measured as it appears early in the apoptosis pathway. As shown in FIG. 23, active caspase 8 was detected as early as 2 hours in HCT116 cells treated with MOC-31 IgG-scTRAIL with a maximum increase of −3.5-fold at 8 hours compared to untreated cells. In TRAIL treated cells, caspase 8 was delayed until 4 hours and only reached 1.5-fold increase at 8 hours compared to untreated cells.

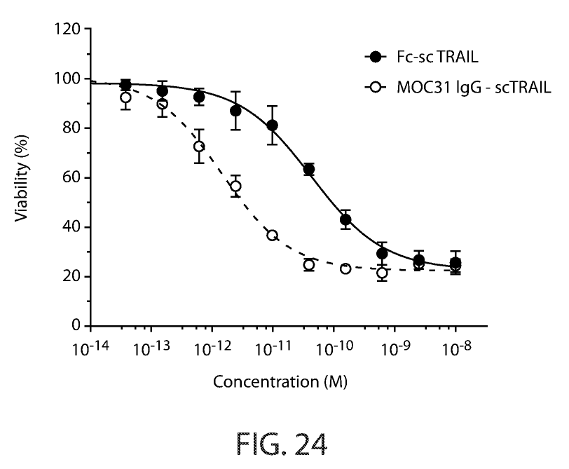

MOC-31 IgG-scTRAIL was also compared to Fc-scTRAIL in a cell viability assay (FIG. 24). Similar with the comparison to TRAIL, the effective concentration (IC₅₀) of apoptosis was significantly improved with MOC-31 IgG-scTRAIL compared to Fc-scTRAIL, however the maximum fraction of cells that underwent apoptosis did not increase.

### EQUIVALENTS

Those skilled in the art will recognize, or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### INCORPORATION BY REFERENCE

The disclosure of each and every U.S. and foreign patent and pending patent application and publication referred to herein is specifically incorporated herein by reference in its entirety, as are the contents of any Sequence Listing and Figures.

1. A single mutant polypeptide chain of an Fc-TRAIL fusion polypeptide comprising two polypeptide chains dimerized by at least one inter-Fc disulfide bond,
the mutant chain comprising a human IgG Fc moiety peptide-bound to a set of three human TRAIL monomer moieties to form a single unbranched polypeptide comprising, in amino- to carboxyl-terminal order, the Fc moiety, a TRAIL-Fc linker, a first TRAIL monomer, an inter-TRAIL monomer linker, a second TRAIL monomer, a second inter-TRAIL monomer linker, and a third TRAIL monomer,
wherein the each linker consists of 15-20 amino acids and each of the two inter-TRAIL monomer linkers comprises 3 G₄S domains, and
wherein at least two of the three TRAIL monomers comprise at least one stabilizing mutation not found in native wild-type human TRAIL, and
wherein, the Fc-TRAIL fusion polypeptide formed by the dimerization of two copies of the mutant polypeptide chain exhibits a melting temperature of greater than or equal to 65° C.

2. The polypeptide chain of claim 1, wherein the at least one stabilizing mutation is at a position corresponding to position 247 of wild-type TRAIL (SEQ ID NO:28) and is an amino acid other than an isoleucine that is located at this position in wild-type TRAIL.

3. The polypeptide chain of claim 2, wherein the amino acid other than the isoleucine is glycine, alanine, valine or leucine.

4. The polypeptide chain of claim 2, wherein the amino acid other than the isoleucine is valine (I247V).

5. The polypeptide chain of claim 1, wherein the at least one stabilizing mutation is selected from R121I, R130G, Y213W, S215D, N228S and I247V.

6. The polypeptide chain of claim 1, wherein the at least one stabilizing mutation comprises a combination of at least two stabilizing mutations selected from the following six combinations:
1) R121I and I247V;
2) N228S and I247V;
3) R130G and I247V;
4) R121I, R130G, Y213W, S215D and I247V;
5) R130G, Y213W, S215D and I247V; and
6) R130G, Y213W, S215D, N228S and I247V.

7. A single mutant polypeptide chain of a TRAIL fusion polypeptide,
the mutant chain comprising a human serum albumin moiety peptide-bound to a set of three human TRAIL monomer moieties to form a single unbranched polypeptide comprising, in amino- to carboxyl-terminal order, an Fc moiety, a TRAIL-Fc linker, a first TRAIL monomer, an inter-TRAIL monomer linker, a second TRAIL monomer, a second inter-TRAIL monomer linker, and a third TRAIL monomer,
wherein the each linker consists of 15-20 amino acids and each of the two inter-TRAIL monomer linkers comprises 3 G₄S domains, and
wherein at least two of the three TRAIL monomers comprises at least one stabilizing mutation not found in native wild-type human TRAIL, and
wherein, the Fc-TRAIL fusion polypeptide formed by the dimerization of two copies of the mutant polypeptide chain exhibits a melting temperature of greater than or equal to 65° C.

8. The polypeptide chain of claim 7, wherein the at least one stabilizing mutation is at a position corresponding to position 247 of wild-type TRAIL (SEQ ID NO:28) and is an amino acid other than an isoleucine that is located at this position in wild-type TRAIL.

9. The polypeptide chain of claim 8, wherein the amino acid other than the isoleucine is glycine, alanine, valine or leucine.

10. The polypeptide chain of claim 8, wherein the amino acid other than the isoleucine is valine (I247V).

11. The polypeptide chain of claim 7, wherein the at least one stabilizing mutation is selected from R121I, R130G, Y213W, S215D, N228S and I247V.

12. The polypeptide chain of claim 7, wherein the at least one stabilizing mutation comprises a combination of at least two stabilizing mutations selected from the following six combinations:
1) R121I and I247V;
2) N228S and I247V;
3) R130G and I247V;
4) R121I, R130G, Y213W, S215D and I247V;
5) R130G, Y213W, S215D and I247V; and
6) R130G, Y213W, S215D, N228S and I247V.

13. A method of treating a cancer in a human patient, the method comprising administering to the patient an effective amount of the Fc-TRAIL fusion polypeptide formed by the dimerization of two copies of the mutant polypeptide chain of claim 1.

14. A polypeptide comprising an amino acid sequence at least 95% identical to amino acid residues 95-281, 114-281, or 120-281 of SEQ ID NO:28, and comprising a substitution at one or more of positions 121, 130, 228, and 247.

15. The polypeptide of claim 14, wherein the polypeptide comprises at least one substitution selected from the group consisting of R121I, R130G, N228S, and I247V.

16. The polypeptide of claim 14, wherein the polypeptide comprises at least one substitution selected from the group consisting of 1247G, I247A, I247V, and I247L.

17. The polypeptide of claim 14, further comprising a substitution at one or both of positions 213 and 215.

18. The polypeptide of claim 14, further comprising at least one substitution selected from the group consisting of Y213W and S215D.

19. The polypeptide of claim 14, comprising a set of substitutions selected from the group consisting of: (i) R121I and I247V; (ii) N228S and 1247V; (iii) R130G and I247V; (iv) R121I, R130G, Y213W, S215D and I247V; (v) R130G, Y213W, S215D and I247V; and (vi) R130G, Y213W, S215D, N228S and I247V.

20. A protein comprising two polypeptide chains, each polypeptide chain comprising a portion of an antibody constant region and a single-chain TRAIL trimer, wherein the protein has a melting temperature greater than about 60° C.

21. The protein of claim 20, wherein each polypeptide chain comprises an amino acid sequence at least 95% identical to amino acid residues 95-281, 114-281, or 120-281 of SEQ ID NO:28, and comprising a substitution at one or more of positions 121, 130, 228, and 247.

22. A protein comprising two polypeptide chains, each polypeptide chain comprising a portion of an antibody constant region and a single-chain TRAIL trimer, wherein the protein retains at least 10% of initial activity after incubation in 90% mouse serum at a final concentration of 1 μM for 7 days at 37° C.

23. The protein of claim 22, wherein each polypeptide chain comprises an amino acid sequence at least 95% identical to amino acid residues 95-281, 114-281, or 120-281 of SEQ ID NO:28, and comprising a substitution at one or more of positions 121, 130, 228, and 247.

24. A protein comprising two polypeptide chains, each polypeptide chain comprising a portion of an antibody constant region and a single-chain TRAIL trimer, wherein the protein has a terminal half-life in mouse circulation of 10 hours or greater.

25. The protein of claim 24, wherein each polypeptide chain comprises an amino acid sequence at least 95% identical to amino acid residues 95-281, 114-281, or 120-281 of SEQ ID NO:28, and comprising a substitution at one or more of positions 121, 130, 228, and 247.

26. The polypeptide chain of claim 1, wherein the at least one stabilizing mutation comprises a combination of stabilizing mutations selected from the group consisting of:
1) R121I, R130G, and I247V;
2) R130G, N228S, and I247V;
3) R121I, R130G, N228S, and I247V;
4) R121I, N228S, and I247V;
5) R121I and R130G;
6) R121I, R130G, and N228S;
7) R121I and N228S; and
8) R130G and N228S.

27. The polypeptide chain of claim 7, wherein the at least one stabilizing mutation comprises a combination of stabilizing mutations selected from the group consisting of:
1) R121I, R130G, and I247V;
2) R130G, N228S, and I247V;
3) R121I, R130G, N228S, and I247V;
4) R121I, N228S, and I247V;
5) R121I and R130G;
6) R121I, R130G, and N228S;
7) R121I and N228S; and
8) R130G and N228S.

28. The polypeptide of claim 14, comprising a set of substitutions selected from the group consisting of:
1) R121I, R130G, and I247V;
2) R130G, N228S, and I247V;
3) R121I, R130G, N228S, and I247V;
4) R121I, N228S, and I247V;
5) R121I and R130G;
6) R121I, R130G, and N228S;
7) R121I and N228S; and
8) R130G and N228S.
